(19) United States
(12) Patent Application Publication
(10) Pub. No.: US 2022/0160714 A1
(43) Pub. Date: May 26, 2022

(54) METHODS FOR TREATING COLORECTAL CANCER

(71) Applicant: ICAHN SCHOOL OF MEDICINE AT MOUNT SINAI, New York, NY (US)

(72) Inventors: Ross CAGAN, New York, NY (US); Erdem BANGI, New York, NY (US)

(21) Appl. No.: 17/442,022

(22) PCT Filed: Mar. 23, 2020
(86) PCT No.: PCT/US2020/024183

§ 371 Date: Sep. 22, 2021

Related U.S. Application Data
(60) Provisional application No. 62/822,453, filed on Mar. 22, 2019.

(51) Int. Cl. A61K 31/519; A61K 31/4523; A61K 31/4184; A61K 31/166; A61K 31/4412; A61K 31/437; A61K 31/44; A61K 31/365; A61K 31/352; A61K 31/18; A61K 31/535; A61K 31/663; A61P 35/00

(52) U.S. Cl. CPC A61K 31/519; A61K 31/4523; A61K 31/4184; A61K 31/166; A61K 31/4412; A61K 31/437; A61P 35/00; A61K 31/365; A61K 31/352; A61K 31/18; A61K 31/535; A61K 31/663; A61K 31/44

(57) ABSTRACT

In one aspect, provided herein are methods for treating colorectal cancer in a human subject, the methods comprising administering to the human subject a composition comprising a mitogen-activated protein kinase kinase (MEK) inhibitor and a composition comprising bisphosphonate. In a particular aspect, provided herein is a method for treating colorectal cancer in a human subject, the method comprising administering to the human subject trametinib dimethyl sulfide or a composition thereof and zoledronic acid or a composition thereof.

This application claims the priority benefit of U.S. Provisional Patent Application Ser. No. 62/822,453, filed Mar. 22, 2019, which is hereby incorporated by reference in its entirety.

### FIELD

In one aspect, provided herein are methods for treating colorectal cancer in a human subject, the methods comprising administering to the human subject a composition comprising a mitogen-activated protein kinase kinase (“MEK”) inhibitor and a composition comprising bisphosphonate. In a particular aspect, provided herein is a method for treating colorectal cancer in a human subject, the method comprising administering to the human subject trametinib dimethyl sulfide or a composition thereof and zoledronic acid or a composition thereof.

### BACKGROUND OF THE INVENTION

Colorectal cancer (“CRC”) remains the second leading cause of cancer mortality in the United States. Current standard of care includes surgery and 5-fluorouracil (“5-FU”)-based chemotherapy combinations such as FOLFIRI (5-FU/leucovorin/irinotecan) and FOLFOX (5-FU/leucovorin/oxaliplatin); recalcitrant or recurrent disease is then treated with one of several targeted therapies. Despite an increasing number of therapeutic options for CRC patients, those diagnosed with metastatic disease (“mCRC”) have a five year survival rate of 11%. Further, toxicities from targeted therapies are substantial: for example, many approved therapies inhibit FLT1, which is closely associated with kidney toxicity and hypertension (Izzedine et al., “Angiogenesis Inhibitor Therapies: Focus on Kidney Toxicity and Hypertension,” Am. J. Kidney Dis. 50:203-218 (2007); Hayman et al., “VEGF Inhibition, Hypertension, and Renal Toxicity,” Curr. Oncol. Rep. 14:285-294 (2012); Skarderud et al., “Efficacy and Safety of Regorafenib in the Treatment of Metastatic Colorectal Cancer: A Systematic Review,” Cancer Treat. Rev.

62:61-73 (2018)).

Tumors with oncogenic RAS isoforms (‘RAS-mutant’ tumors) represent a particular challenge. An estimated 30%-50% of colorectal cancer patient tumors include an oncogenic KRAS mutation; an additional ˜6% of colorectal tumors contain mutations in NRAS or HRAS (Chang et al., “Mutation Spectra of RAS Gene Family in Colorectal Cancer,” Am. J. Surg. 212:537-544 e533 (2016); Valtorta et al., “KRAS Gene Amplification in Colorectal Cancer and Impact on Response to EGFR-targeted Therapy,” Int. J. Cancer 133:1259-1265 (2013)). Several studies—though not all—have associated RAS-mutant tumors with more aggressive metastatic disease and reduced survival (Jones et al., “Specific Mutations in KRAS Codon 12 are Associated with Worse Overall Survival in Patients with Advanced and Recurrent Colorectal Cancer,” Br. J. Cancer 116:923-929 (2017); Karagkounis et al., “Incidence and Prognostic Impact of KRAS and BRAF Mutation in Patients Undergoing Liver Surgery for Colorectal Metastases,” Cancer 119:4137-4144 (2013); Kim et al., “The Impact of KRAS Mutations on Prognosis in Surgically Resected Colorectal Cancer Patients with Liver and Lung Metastases: A Retrospective Analysis,” BMC Cancer 16:120 (2016); Russo et al., “Mutational Analysis and Clinical Correlation of Metastatic Colorectal Cancer,” Cancer 120:1482-1490 (2014); Umeda et al., “Poor Prognosis of KRAS or BRAF Mutant Colorectal Liver Metastasis without Microsatellite Instability,” J. Hepatobiliary Pancreat. Sci. 20:223-233 (2013)). RAS-mutant CRC affects more than 60,000 patients annually, leading to more than 20,000 cancer deaths in the U.S. alone (Andreyev et al., “Kirsten Ras Mutations in Patients with Colorectal Cancer: The ‘RASCAL II’ Study,” Br. J. Cancer 85:692-696 (2001); Ostrem et al., “K-Ras(G12C) Inhibitors Allosterically Control GTP Affinity and Effector Interactions,” Nature 503:548-551 (2013)). More broadly, despite recent advances (Ostrem et al., “K-Ras(G12C) Inhibitors Allosterically Control GTP Affinity and Effector Interactions,” Nature 503:548-551 (2013); Lim et al., “Therapeutic Targeting of Oncogenic K-Ras by a Covalent Catalytic Site Inhibitor,” Angew Chem. Int. Ed. Engl. 53:199-204 (2014); Zimmermann et al., “Small Molecule Inhibition of the KRAS-PDEdelta Interaction Impairs Oncogenic KRAS Signaling,” Nature 497:638-642 (2013)) therapeutic options for targeting RAS-dependent cancers remain limited (Misale et al., “Emergence of KRAS Mutations and Acquired Resistance to Anti-EGFR Therapy in Colorectal Cancer,” Nature 486:532-536 (2012); Nazarian et al., “Melanomas Acquire Resistance to B-RAF(V600E) Inhibition by RTK or N-RAS Upregulation,” Nature 468:973-977 (2010); Stephen et al., “Dragging Ras Back in the Ring,” Cancer Cell 25:272-281 (2014)).

FDA-approved therapies that target the RAS pathway have shown limited efficacy in patients with KRAS-mutant mCRC. For example, the FDA-approved kinase inhibitor regorafenib (Stivarga) provides limited mCRC patient survival benefit (1.4-2.5 months) with substantial and highly penetrant adverse events (Skarderud et al., “Efficacy and Safety of Regorafenib in the Treatment of Metastatic Colorectal Cancer: A Systematic Review,” Cancer Treat. Rev. 62:61-73 (2018)). Other FDA-approved RAS pathway inhibitors such as trametinib (Mekinist) as well as immune checkpoint inhibitors have failed in CRC clinical trials for microsatellite stable disease, (Falchook et al., “Activity of the Oral MEK Inhibitor Trametinib in Patients with Advanced Melanoma: A Phase 1 Dose-escalation Trial,” Lancet Oncol. 13:782-789 (2012); Infante et al., “Safety, Pharmacokinetic, Pharmacodynamic, and Efficacy Data for the Oral MEK Inhibitor Trametinib: A Phase 1 Dose-escalation Trial,” Lancet Oncol. 13:773-781 (2012)) leading to new interest in combinations of targeted therapies (Lee et al., “Efficacy of the Combination of MEK and CDK4/6 Inhibitors In Vitro and In Vivo in KRAS Mutant Colorectal Cancer Models,” Oncotarget 7:39595-39608 (2016); Martinelli et al., “Cancer Resistance to Therapies Against the EGFR-RAS-RAF Pathway: The Role of MEK,” Cancer Treat. Rev. 53:61-69 (2017)). KRAS-mutant mCRC patients—typically presenting with right-sided tumors that are more aggressive on recurrence—are resistant to or even harmed by therapies targeting EGFR, and testing for RAS mutations are standard exclusionary criteria (Benvenuti et al., “Oncogenic Activation of the RAS/RAF Signaling Pathway Impairs the Response of Metastatic Colorectal Cancers to Anti-epidermal Growth Factor Receptor Antibody Therapies,” Cancer Res. 67:2643-2648 (2007); Nicolantonio et al., “Wild-type BRAF is Required for Response to Panitumumab or Cetuximab in Metastatic Colorectal Cancer,” J. Clin. Oncol. 26:5705-5712 (2008); Gong et al., “RAS and BRAF in Metastatic Colorectal Cancer Management,” J. Gastrointest. Oncol. 7:687-704 (2016); Lievre et al., “KRAS Mutation Status is Predictive of Response to Cetuximab Therapy in Colorectal Cancer,” Cancer Res. 66:3992-3995 (2006); Benson et al., “NCCN Guidelines Insights: Colon Cancer, Version 2.2018,” J. Nat'l. Compr. Canc. Netw. 16:359-369 (2018)). Overall, KRAS mCRC patients with recurrent disease have few good therapeutic options.

Zoledronate and related bisphosphonates are associated with strong protection against colorectal cancer: women who took bisphosphonates to protect from excess bone resorption—breast cancer patients, postmenopausal women—exhibited a 40-59% reduced incidence of CRC (Pazianas et al., “Reduced Colon Cancer Incidence and Mortality in Postmenopausal Women Treated with an Oral Bisphosphonate—Danish National Register Based Cohort Study,” Osteoporos Int. 23:2693-2701 (2012); Rennert et al., “Use of Bisphosphonates and Reduced Risk of Colorectal Cancer,” J. Clin. Oncol. 29:1146-1150 (2011)).

Accordingly, there remains a need for therapeutic agents to treat colorectal cancer, in particular, KRAS-mutant metastatic colorectal cancer.

The present invention is directed to overcoming these and other deficiencies in the art.

### SUMMARY OF THE INVENTION

In one aspect, provided herein are methods for treating colorectal cancer, the method comprising administering to a human subject in need thereof a mitogen-activated protein kinase/extracellular signal-regulated kinase (“MAPK/ERK”) kinase (MEK) inhibitor and a bisphosphonate. In a specific embodiment, provided herein is a method for treating colorectal cancer, the method comprising administering to a human subject diagnosed with colorectal cancer a first composition comprising a mitogen-activated protein kinase/extracellular signal-regulated kinase (MAPK/ERK) kinase (MEK) inhibitor and a second composition comprising a bisphosphonate. The first and second compositions may be administered by the same or different routes of administration. In a specific embodiment, the first composition is administered to the subject orally (e.g., as a tablet). In another specific embodiment, the second composition is administered to the subject intravenously or orally. In addition, the first and second compositions may be administered concurrently. The first composition may be administered daily and the second composition may be administered daily, every 2 days, every 3 days, once a week, once every two weeks, once every three weeks, or once every four weeks. In a specific embodiment, the dosage of the MEK inhibitor and the dosage of the bisphosphonate used to treat colorectal cancer in accordance with the methods described herein are the dosages approved by the federal Food and Drug Administration for any use. In other embodiments, the dosage of the MEK inhibitor and dosage of the bisphosphonate used to treat colorectal cancer in accordance with the methods described herein are lower than the dosages approved by the U.S. Food and Drug Administration for any use.

In another aspect, provided herein are a first composition and a second composition for use in a method for treating colorectal cancer in a human subject, wherein the first composition comprises a MEK inhibitor and the second composition comprises a bisphosphonate. The first and second compositions may be administered by the same or different routes of administration. In a specific embodiment, the first composition is administered to the subject orally (e.g., as a tablet). In another specific embodiment, the second composition is administered to the subject intravenously or orally. In addition, the first and second compositions may be administered concurrently. The first composition may be administered daily and the second composition may be administered daily, every 2 days, every 3 days, once a week, once every two weeks, once every three weeks, or once every four weeks. In a specific embodiment, the dosage of the MEK inhibitor and the dosage of the bisphosphonate used to treat colorectal cancer in accordance with the methods described herein are the dosages approved by the federal Food and Drug Administration for any use. In other embodiments, the dosage of the MEK inhibitor and dosage of the bisphosphonate used to treat colorectal cancer in accordance with the methods described herein are lower than the dosages approved by the federal Food and Drug Administration for any use.

In a specific embodiment, the MEK inhibitor used to treat cancer in accordance with the methods described herein is trametinib. In another specific embodiment, the MEK inhibitor used to treat cancer in accordance with the methods described herein is trametinib dimethyl sulfoxide. In another specific embodiment, the first composition used to treat cancer in accordance with the methods described herein is MEKINIST®.

In another specific embodiment, the MEK inhibitor used to treat cancer in accordance with the methods described herein is cobimetinib. In a specific embodiment, the MEK inhibitor used to treat cancer in accordance with the methods described herein is cobimetinib fumarate. In another specific embodiment, the first composition used to treat cancer in accordance with the methods described herein is COTELLIC®.

In another specific embodiment, the MEK inhibitor used to treat cancer in accordance with the methods described herein is binimetinib. In another specific embodiment, the first composition used to treat cancer in accordance with the methods described herein is MEKTOVI®.

In another specific embodiment, the MEK inhibitor used to treat cancer in accordance with the methods described herein is CI-1040 (PD184352), PD0325901, Selumetinib (AZD6244), MEK162, AZD8330, TAK-733, GDC-0623, Refametinib (RDEA119; BAY 869766), Pimasertib (AS703026), R04987655 (CH4987655), R05126766, WX-554, HL-085, E6201, GDC-0623, or PD098059.

In a specific embodiment, the bisphosphanonate used to treat cancer in accordance with the methods described herein is etidronate, alendronate, risedronate, ibandronate, zoledronic acid, alendronate sodium, clodronate, tiludronate, pamidronate, neridronate, or olpadronate. In another specific embodiment, the bisphosphonate used to treat cancer in accordance with the methods described herein is zoledronic acid. In another specific embodiment, the second composition used to treat cancer in accordance with the methods described herein is Zometa®.

In another specific embodiment, the bisphosphonate used to treat cancer in accordance with the methods described herein is ibandronate. In another specific embodiment, the second composition used to treat cancer in accordance with the methods described herein is BONIVA®.

In some embodiments, the colorectal cancer treated in accordance with the methods described herein is KRAS-mutant colorectal cancer, NRAS-mutant colorectal cancer, or HRAS-mutant colorectal cancer. In a specific embodiment, the colorectal cancer treated in accordance with the methods described herein is KRAS-mutant colorectal cancer. In another specific embodiment, the colorectal cancer treated in accordance with the methods described herein is KRAS-mutant colorectal adenocarcinoma cancer. In certain embodiments, the colorectal cancer treated in accordance with the methods described herein contains a gene isoform previously demonstrated to activate KRAS, HRAS, or NRAS. In a specific embodiment, the unresponsive to other therapies approved for colorectal cancer.

### BRIEF DESCRIPTION OF THE DRAWINGS

FIGS. 1A-D illustrate an overview of one embodiment of the construction of a Drosophila patient model. FIG. 1A is an outline of the approach. First, a comprehensive genomic analysis of the patient's tumor and normal DNA (copy number, whole exome sequencing, and targeted HotSpot panel) was performed. Then, a personalized Drosophila model that captures a portion of the patient tumor's genomic complexity was generated by targeting each Drosophila ortholog specifically in the Drosophila hindgut. After the model was validated, a high throughput ‘rescue from lethality’ drug screen was performed on FDA-approved drugs as single agents and in combination. Findings were then presented to a multidisciplinary tumor board (MTB). A personalized treatment plan based on the MTB's recommendation was prepared and IRB approved, followed by patient treatment. FIG. 1B shows a patient's genomic landscape: Genes altered in the patient's tumor, their functions and Drosophila orthologs are indicated. LOH: copy number neutral loss of heterozygosity. FIG. 1C shows a GAL4/UAS system used for targeted genetic manipulations in Drosophila. Transgenes targeting nine genes (ras85D^{G12V}, etc.) were cloned downstream of a GAL4 responsive UAS promoter and transgenic flies were generated. Transgene expression were then induced in a tissue-specific manner by crossing transgenic flies to byn-gal4 for colon epithelium, tubulin-gal4 for ubiquitous expression. FIG. 1D shows a personalized construct generated for the patient, targeting 9 genes. This construct expressed a GAL4-inducible (i)UAS-ras85D^{G12V} transgene and (ii) synthetic 8-hairpin cluster targeting the Drosophila orthologs of the 8 tumor suppressor genes. After transgenic flies were generated, transgenic constructs UAS-ago^{RNAi} and UAS-apc^{RNAi} were genetically introduced by standard genetic crosses to increase overall ago and apc knockdown.

FIGS. 2A-D show validating and screening a Drosophila patient model. FIG. 2A shows that expressing byn>GFP in control animals highlighted the hindgut in brightfield (top panels) and expression of the byn-GAL4 driver specific to the hindgut (bottom panels). 5× and 10× microscope magnifications are shown. FIG. 2B shows expressing the CPCT-006.1 transgene set in the hindgut led to strong expansion of the anterior hindgut. The midgut/hindgut (M/H) boundaries are indicated; the dark regions in the CPCT-006 brightfield images likely reflect cell death. Bars represent 100 μM; image contrast enhanced equally by Preview software for clarity. FIGS. 2C-D show Trametinib in combination with ibandronate or zoledronate rescued the lethality observed by the patient's personalized Drosophila model. Concentrations indicate final food concentrations. Each data point represents a replicate with 10-15 experimental and 20-30 control animals. Raw numbers are provided in Table 4C. Error bars indicate standard error of the mean.

FIGS. 3A-C show the results of secondary assays of drug response. FIG. 3A shows the results of a Western blot analysis of MAPK signaling pathway output from control and drug treated hindgut lysates using dually phosphorylated ERK (dpERK) as a readout. Quantification represents two independent experiments with different sets of biological replicates. Each experiment was performed in triplicate with 10 hindguts/biological replicate. (Gel images are shown in FIG. 6C). FIGS. 3B-3C show analysis of the expansion of the anterior hindgut in control and drug-treated animals. FIG. 3B shows quantification of the anterior region of the hindgut. Data points indicate individual hindguts. FIG. 3C shows two images representing the high and low ends of the size distribution observed in the assay. Quantified region of the hindgut is outlined by white dashed lines. T: 1 μM trametinib, Z: 0.7 μM zoledronate in the food. Statistical significance in panels A and B was determined using multiple t-tests with Holm Sidak correction for multiple hypotheses.

FIGS. 4A-B show the results of patient response. FIG. 4A shows patient scans pre-treatment and 27 weeks post-treatment. The arrow indicates example of lesion in left supraclavicular node. FIG. 4B shows two examples of target lesion shrinkage at indicated time points highlighted by shading plus dashed outline; the upper panels provide detail to FIG. 4A.

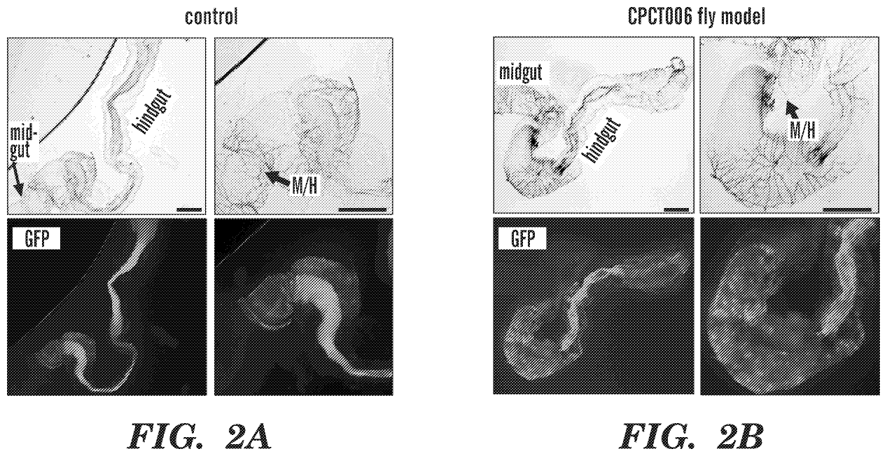

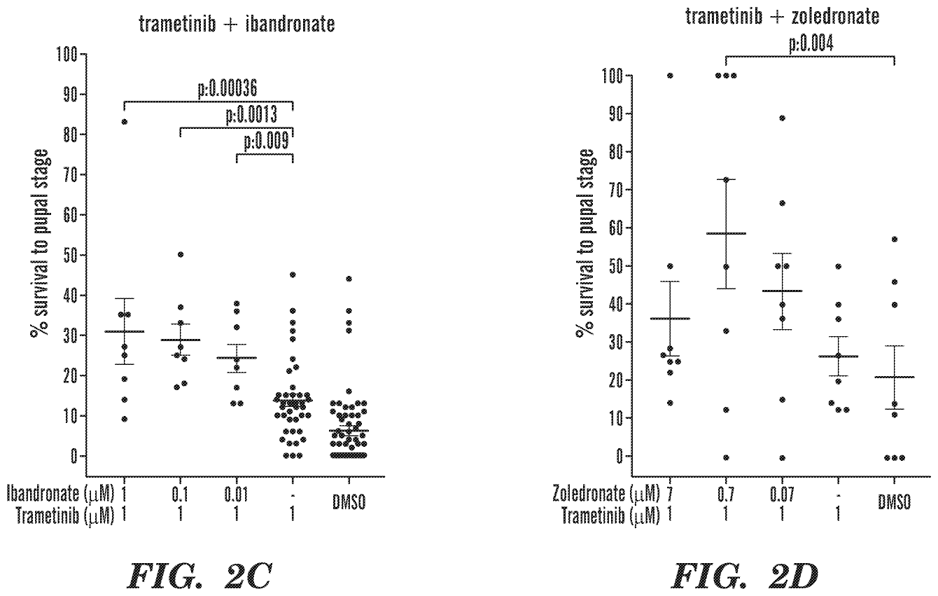

FIG. 2. 5A-C show validation of a patient's personalized Drosophila model (see examples infra for details). FIG. 5A shows a qPCR analysis of knockdown profiles for 7 genes in the synthetic cluster. Human orthologs of genes indicated in parentheses. FIG. 5B shows p53 knockdown at the protein level measured by Western blot analysis. FIG. 5C shows MAPK signaling pathway output using dually phosphorylated ERK (dpERK) by Western blot analysis. Experiments were performed in triplicate with 6 animals/biological replicate.

FIGS. 6A-C show validation of patient's personalized Drosophila model and drug response from hindgut lysates (see examples infra for details). FIGS. 6A-B show Western blot analysis of knockdown for two genes in the synthetic cluster from hindgut lysates at the protein level. FIG. 6C shows Western blot analysis of MAPK signaling pathway output in two independent experiments using different sets of biological replicates. Experiments were performed in triplicate with 10 hindguts/biological replicate.

FIG. 7 is a graph showing the effect of Trametinib plus Zoledronate on two separate KRAS-mutant colorectal cancer cell lines, DLD-1 and HCT-116. DMSO and regorafenib (regoraf) were used as controls. Zoledronate (zoledr or zol) or trametinib (tra or tramet) were used separately and together. Together, the two drugs showed strongly increased killing of both cell types. The Y-axis represents % cell viability in cell culture.

### DETAILED DESCRIPTION OF THE INVENTION

In one aspect, provided herein are mitogen-activated protein kinase/extracellular signal-regulated kinase (MAPK/ERK) kinase (MEK) inhibitors and bisphosphonates for use in the treatment of colorectal cancer. In a specific embodiment, a composition comprising a MEK inhibitor and a composition comprising a bisphosphonate are used to treat colorectal cancer of a human subject.

In one aspect, provided herein is a method for treating colorectal cancer in a human subject, the method comprising administering to the human subject a first composition comprising a MAPK/ERK kinase (MEK) inhibitor and a second composition comprising a bisphosphonate. In a specific embodiment, provided herein is a method for treating colorectal cancer, the method comprising administering to a human subject diagnosed with colorectal cancer, a first composition comprising a MEK inhibitor and a second composition comprising a bisphosphonate. In another specific embodiment, provided herein is method for treating colorectal cancer, the method comprising administering to a human subject diagnosed with colorectal cancer, an effective amount of a first composition comprising a MEK inhibitor and an effective amount of a second composition comprising a bisphosphonate.

In some embodiments, a first composition comprising a MEK inhibitor and a second composition comprising a bisphosphonate are administered to the human subject to treat colorectal cancer by the same route of administration. For example, the first and second compositions may be administered orally. In other embodiments, a first composition comprising a MEK inhibitor and a second composition comprising bisphosphonate are administered by different routes of administration. For example, the first composition may be administered orally to treat the human subject and the second composition may be administered intravenously to treat the human subject. In a specific embodiment, one, two, or more of the inactive ingredients identified in Table 1 or Table 2, infra, may be included in a composition described herein. In a specific embodiment, a composition comprising a MEK inhibitor is a pharmaceutical composition. In another specific embodiment, a composition comprising a bisphosphonate is a pharmaceutical composition. In a specific embodiment, a composition (e.g., a pharmaceutical composition) comprising a MEK inhibitor contains the MEK inhibitor as the sole active ingredient and all other ingredients in the composition are inactive. In another specific embodiment, a composition (e.g., a pharmaceutical composition) comprising a bisphosphonate contains the bisphosphonate as the sole active ingredient and all ingredients in the composition are inactive ingredients. Examples of inactive ingredients include pharmaceutically acceptable excipients, carriers, and stabilizers. In addition, thickening, lubricating, and coloring agents may be included in a composition described herein. In specific embodiments, the ingredients included in a composition described herein are sterile when administered to a subject. Examples of carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); water; saline; gelatin; starch paste; talc; keratin; gum acacia; sodium stearate; sodium chloride; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some embodiments, a MEK inhibitor used in accordance with the methods described herein is a reversible inhibitor of mitogen-activated protein kinase/extracellular signal-regulated kinase (MAPK/ERK) kinase 1 (MEK1) or MEK 2. In particular embodiments, a MEK inhibitor used in accordance with the methods described herein is a reversible inhibitor of MEK 1 and MEK 2 activation and of MEK 1 and MEK 2 kinase activity. In a specific embodiment, the MEK inhibitor used in accordance with the methods described herein is trametinib. In a another specific embodiment, the MEK inhibitor used in accordance with the methods described herein is trametinib dimethyl sulfoxide. In another specific example, the MEK inhibitor used in accordance with the methods described herein is cobimetinib. In a specific embodiment, the MEK inhibitor used in accordance with the methods described herein is cobimetinib fumarate. In a specific embodiment, the MEK inhibitor used in accordance with the methods described herein is binimetinib.

In some embodiments, a MEK inhibitor used in accordance with the methods described herein is CI-1040 (PD184352), PD0325901, Selumetinib (AZD6244), MEK162, AZD8330, TAK-733, GDC-0623, Refametinib (RDEA119; BAY 869766), Pimasertib (AS703026), R04987655 (CH4987655), R05126766, WX-554, HL-085, E6201, GDC-0623, or PD098059. In some embodiments, the first composition comprising a MEK inhibitor which is used in accordance with the methods described herein, is one discussed in Table 1, infra.

In a specific embodiment, the first composition comprising a MEK inhibitor used in accordance with the methods described herein, is MEKINIST®. In another specific embodiment, the first composition comprising a MEK inhibitor, which is used in accordance with the methods described herein, is COTELLIC®. In another specific embodiment, the first composition comprising a MEK inhibitor, which is used in accordance with the methods described herein, is MEKTOVI®.

Bisphosphonates are a well-known class of drugs that have been used, e.g., to prevent the loss of bone density and to treat osteoporosis and similar diseases. Bisphosphonates, which are sometimes referred to as diphosphonates because they have two phosphonate (PO(OH)₂) groups, include for example etidronate, alendronate, risedronate, ibandronate, zoledronic acid, alendronate sodium, clodronate, tiludronate, pamidronate, neridronate, and olpadronate. In a specific embodiment, a bisphosphonate used in accordance with the methods described herein, is one of those identified in the foregoing sentence.

In some embodiments, the bisphosphonate used in accordance with the methods described herein is a non-nitrogenous containing bisphosphonate, such as, e.g., etidronate, clodronate or tiludronate. In other embodiments, the bisphosphonate used in accordance with the methods described herein, is a nitrogenous-containing bisphosphonate, such as, e.g., pamidronate, neridonate, olpadronate, alendronate, ibandronate, riserdronate, or zoledronate. In a specific embodiment, a bisphosphonate is selected for use in accordance with the methods described herein, is less toxic, is associated with fewer side effects or both.

In a specific embodiment, the bisphosphonate used in accordance with the methods described herein, is zoledronic acid. In another specific embodiment, the bisphosphonate used in accordance with the methods described herein is ibandronate.

In some embodiments, the second composition comprising bisphosphonate, which is used in accordance with the methods described herein, is one described in Table 2, infra. In a specific embodiment, the second composition comprising bisphosphonate, which is used in accordance with the methods described herein, is Zometa®. In another specific embodiment, the second composition comprising bisphosphonate, which is used in accordance with the methods described herein, is Boniva®.

In some embodiments, the specific MEK inhibitor and the specific bisphosphonate used to treat colorectal cancer in accordance with the methods described herein are the MEK inhibitor and bisphosphonate that increased survival of a fly avatar of colorectal cancer. In a specific embodiment, a fly avatar of colorectal cancer, such as described in International Patent Application Publication No. WO 2017/117344 A1 and U.S. Patent Application Publication No. 2019/0011435 A1 (each of which is incorporated herein by reference in its entirety) is used to identify the specific MEK Inhibitor and the specific bisphosphonate that are used to treat colorectal cancer in accordance with the methods described herein. In another specific embodiment, a personalized fly avatar of colorectal cancer generated such as described in Examples 1 and 3, infra, is used to identify the specific MEK Inhibitor and the specific bisphosphonate that are used to treat colorectal cancer in accordance with the methods described herein. In another specific embodiment, a generic avatar of colorectal cancer or an avatar army for colorectal cancer generated such as described in U.S. Patent Application Publication No. 2019/0011435 A1 or International Patent Application Publication No. WO 2017/117344 A1, each of which is incorporated herein by reference in its entirety, is used to identify the specific MEK inhibitor and the specific bisphosphonate that is used to treat colorectal cancer in accordance with the methods described herein.

In some embodiments, provided herein is a method of treating colorectal cancer, the method comprising administering to a human subject in need thereof a MEK inhibitor and a bisphosphonate, wherein the MEK inhibitor and the bisphosphonate were identified in a fly avatar, such as described infra, or as described in U.S. Patent Application Publication No. 2019/0011435 A1 or International Patent Application Publication No. WO 2017/117344 A1, each of which is incorporated herein by reference in its entirety. In a particular embodiment, the MEK inhibitor and bisphosphonate for use in the treatment of colorectal cancer in accordance with the methods described herein resulted in increased survival of a fly avatar of colorectal cancer, such as described herein, or in U.S. Patent Application Publication No. 2019/0011435 A1 or International Patent Application Publication No. WO 2017/117344 A1, each of which is incorporated herein by reference in its entirety.

In some embodiments, a fly avatar of colorectal cancer such as described herein, or in U.S. Patent Application Publication No. 2019/0011435 A1 or International Patent Application Publication No. WO 2017/117344 A1, each of which is incorporated herein by reference in its entirety, is used to confirm the MEK inhibitor and bisphosphonate for use in accordance with the methods described herein for treating colorectal cancer.

In certain embodiments, colorectal cancer cells (e.g., colorectal cancer cell lines or colorectal cancer cells obtained from a human subject) are used to identify the MEK inhibitor and bisphosphonate to use in accordance with the methods described herein. In some embodiments, colorectal cancer cells (e.g., colorectal cancer cell lines or colorectal cancer cells obtained from a human subject) are used to confirm the MEK inhibitor and bisphosphonate to use in accordance with the methods described herein. In a specific embodiment, the colorectal cancer cells are from the human subject intended to be treated or being treated in accordance with the methods described herein.

In certain embodiments, patient-derived xenografts in which colorectal cancer cells from a patient's colorectal cancer or a biopsy of a patient's colorectal cancer is implanted into an immunodeficient or humanized mouse, may be used to identify the MEK inhibitor and bisphosphonate to use in accordance with the methods described herein. In some embodiments, a patient-derived xenograft may be used to confirm the MEK inhibitor and bisphosphonate to use in accordance with the methods described herein.

In certain embodiments, a colorectal cancer animal model (e.g., genetically engineered mouse model or other colorectal cancer animal model) may be used to identify the MEK inhibitor and bisphosphonate to use in accordance with the methods described herein. In some embodiments, a colorectal cancer animal model (e.g., induced germline mutation models and genetically modified mice) may be used to confirm the MEK inhibitor and bisphosphonate to use in accordance with the methods described herein. See, e.g., Johnson and Fleet, Cancer Metastasis Rev. 32: 39-61 (2013), De-Souza and Costa-Casagrande, Arg. Bra. Cir. Dig 31(2):e1369 (2018), and Caetano-Oliveria et al., Pathophysiologically 25:89-99 (2018), each of which is incorporated herein by reference in its entirety, for examples of animal models of colorectal cancer.

In certain embodiments, the specific MEK and the specific bisphosphonate are tested in a fly avatar on colorectal cancer cells, or an animal model for colorectal cancer (e.g., a patient-derived xenograft, genetically modified mouse model or other animal model prior to administration to a human subject.

In a specific embodiment, the MEK inhibitor and the bisphosphonate are each formulated for administration for the intended route of administration. For example, a composition comprising a MEK inhibitor may be formulated for oral administration, intravenous administration, intramuscular administration, subcutaneous administration of any other route. In a specific embodiment, a composition comprising a MEK inhibitor is formulated for oral administration. In another example, a composition comprising a bisphosphonate may be formulated for oral administration, intravenous administration, intramuscular administration, subcutaneous administration, or any other route. In a specific embodiment, a composition comprising a bisphosphonate may be formulated for intravenous administration or oral administration. Examples of formulations for oral administration include a tablet, a capsule, a solution, a dispersion, and a suspension. Examples of formulations for intravenous administration include a liquid solution or suspension, solid forms suitable for solution or suspension in liquid prior to injection, or as emulsions.

In certain embodiments, the dosages of a MEK inhibitor administered to a human patient to treat colorectal cancer in accordance with the methods described herein is a dosage approved by a regulatory agency (e.g., a dosage approved by the FDA) for any approved use. In a specific embodiment, the dosage of a MEK inhibitor administered to a human patient to treat colorectal cancer in accordance with the methods described herein is a dosage provided in Table 1, infra, for the particular MEK inhibitor.

In some embodiments, the frequency of administration of a dose of a MEK inhibitor to a human patient to treat colorectal cancer is a frequency approved by a regulatory agency (e.g., FDA) for any use. In specific embodiments, the frequency of administration of a dose of a MEK inhibitor to a human patient to treat colorectal cancer in accordance with the methods described herein is a dosage provided in Table 1, infra, for the particular MEK inhibitor.

In certain embodiments, the dosage of a MEK inhibitor administered to a human patient to treat colorectal cancer in accordance with the methods described herein is a dosage lower than the dosage approved by a regulatory agency (e.g., a dosage approved by the FDA) for any approved use. In a specific embodiment, the dosage of a MEK inhibitor administered to a human patient to treat colorectal cancer in accordance with the methods described herein is a frequency lower the dosage provided in Table 1, infra, for the particular MEK inhibitor.

In some embodiments, the frequency of administration of a dose of a MEK inhibitor to a human patient to treat colorectal cancer is lower than the frequency approved by a regulatory agency (e.g., the FDA) for any use. In specific embodiments, frequency of administration of a MEK inhibitor to a human patient to treat colorectal cancer in accordance with the methods described herein is a frequency lower than the frequency provided in Table 1, infra, for the particular MEK inhibitor.

In certain embodiments, the dosage of a MEK inhibitor administered to a human patient to treat colorectal cancer in accordance with the methods described herein is dosage greater than the dosage approved by a regulatory agency (e.g., a dosage approved by the FDA) for any approved use. In a specific embodiment, the dosage of a MEK inhibitor administered to a human patient to treat colorectal cancer in accordance with the methods described herein is a dosage greater the dosage provided in Table 1, infra, for the particular MEK inhibitor.

In some embodiments, the frequency of administration of a dose of a MEK inhibitor to a human patient to treat colorectal cancer is greater than the frequency approved by a regulatory agency (e.g., the FDA) for any use. In specific embodiments, the frequency of administration of a MEK inhibitor administered to a human patient to treat colorectal cancer in accordance with the methods described herein is greater than the frequency provided in Table 1, infra, for the particular MEK inhibitor.

In a specific embodiment, the dosage of a MEK inhibitor administered to a subject to treat colorectal cancer in accordance with the methods described herein is a standard of care dosage. See Table 1, infra, for examples of standard care dosages for MEK inhibitors.

In a specific embodiment, the dosage of a MEK inhibitor administered to a subject to treat colorectal cancer in accordance with the methods described herein is generally lower than the dosages that are administered in a standard of care dosage.

In a specific embodiment, the dosage of a MEK inhibitor administered to a subject to treat colorectal cancer in accordance with the methods described herein is generally greater than the dosages that are administered as standard of care dosage. In another specific embodiment, the dosage of a MEK inhibitor administered to a subject to treat colorectal cancer in accordance with the methods described herein is generally for longer periods of time than those described in a standard of care dosage

In some embodiments, the frequency of administration of the MEK inhibitor ranges from once a day up to about once every eight weeks. In specific embodiments, the frequency of administration of the MEK inhibitor ranges from once a day, twice a day, once three times a day, every other day, once every three days, once a week, or once every other week. See Table 1, infra, for examples of the frequency of administration particularly MEK inhibitors.

In some embodiments, a dosage of a MEK inhibitor administered to a human subject to treat colorectal cancer in accordance with the methods described herein is in the range of 0.01 to 25 mg/kg, and more typically, in the range of 0.1 mg/kg to 10 mg/kg, of the subject's body weight. In one embodiment, a dosage administered to a human subject is in the range of about 0.1 mg/kg to about 1 mg/kg, about 0.1 mg/kg to about 1.5 mg/kg, about 0.1 mg/kg to about 2 mg/kg, about 0.1 mg/kg to about 2.5 mg/kg, about 0.1 mg/kg to about 3 mg/kg, about 0.1 mg/kg to about 3.5 mg/kg, about 0.1 mg/kg to about 4 mg/kg, about 0.1 mg/kg to about 4.5 mg/kg, about 0.1 mg/kg to about 5 mg/kg, about 0.1 mg/kg to about 5.5 mg/kg, about 0.1 mg/kg to about 6 mg/kg, about 0.1 mg/kg to about 6.5 mg/kg, about 0.1 mg/kg to about 7 mg/kg, about 0.1 mg/kg to about 7.5 mg/kg, about 0.1 mg/kg to about 8 mg/kg, about 0.1 mg/kg to about 8.5 mg/kg, about 0.1 mg/kg to about 9 mg/kg, about 0.1 mg/kg to about 9.5 mg/kg, about 0.1 mg/kg to about 10 mg/kg, about 0.1 mg/kg to about 15 mg/kg, or about 1 mg/kg to about 10 mg/kg, of about 0.1 mg/kg to about 25 mg/kg, or about 1 mg/kg to about 25 mg/kg, of the human subject's body weight.

In a specific embodiment, a MEK inhibitor is administered to a human subject to treat colorectal cancer in accordance with the methods described herein at a dosage of is 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, or about 25 mg/kg, of the human subject's body weight.

In another specific embodiment, a dosage of a MEK inhibitor administered to a subject to treat colorectal cancer in accordance with the methods described herein is a unit dose of 0.1 mg to 1000 mg or 1 mg to 500 mg. In specific embodiments, a dosage of a MEK inhibitor administered to a subject to treat colorectal cancer in accordance with the methods described herein is a unit dose of 0.1 mg to 900 mg, 0.1 mg to 800 mg, 0.1 mg to 700 mg, 0.1 mg to 600 mg, 0.1 mg to 500 mg, 0.1 mg to 400 mg, 0.1 mg to 300 mg, 0.1 mg to 200 mg, 0.1 mg to 100 mg. In another specific embodiment, a dosage of a MEK inhibitor administered to a subject to treat colorectal cancer in accordance with the methods described herein is a unit dose of 0.1 mg to 75 mg. In another specific embodiment, a dosage of a MEK inhibitor administered to a subject to treat colorectal cancer in accordance with the methods described herein is a unit dose of 1 mg to 200 mg, 1 mg to 175 mg, 1 mg to 150 mg, 1 mg to 125 mg, 1 mg to 100 mg, 1 mg to 80 mg, 1 mg to 75 mg, 1 mg to 70 mg, 1 to 60 mg, 1 to 65 mg, 1 mg to 55 mg, 1 mg to 50 mg, or 1 mg to 45 mg. In a specific embodiment, the dosage of a MEK inhibitor administered to a subject to treat colorectal cancer in accordance with the methods described herein is a unit dose of 1 mg to 40 mg, 1 mg to 35 mg, 1 mg to 30 mg, 1 mg to 25 mg, 1 mg to 20 mg, 1 mg to 15 mg, 1 mg to 10 mg, 1 mg to 5 mg, or 1 mg to 2 mg.

In a specific embodiment, a dosage of a MEK inhibitor is administered in the range of 0.01 to 10 g/m², and more typically, in the range of 0.1 g/m² to 7.5 g/m², of the subject's body weight. In one embodiment, a dosage administered to a human subject is in the range of 0.5 g/m² to 5 g/m², or 1 g/m² to 5 g/m² of the human subject's body's surface area. In a specific embodiment, the dosage of a MEK inhibitor administered to a subject in accordance with the methods described herein is one provided in the examples infra.

In certain embodiments, the dosage of a bisphosphonate administered to a human patient to treat colorectal cancer in accordance with the methods described herein is a dosage approved by a regulatory agency (e.g., a dosage approved by the FDA) for any approved use. In a specific embodiment, the dosage of a bisphosphonate administered to a human patient to treat colorectal cancer in accordance with the methods described herein is a dosage provided in Table 2, infra, for the particular bisphosphonate.

In some embodiments, the frequency of administration of a dose of a bisphosphonate to a human patient to treat colorectal cancer is a frequency approved by a regulatory agency (e.g., the FDA) for any use. In specific embodiments, the frequency of administration of a dose of a bisphosphonate administered to a human patient to treat colorectal cancer in accordance with the methods described herein is a dosage provided in Table 2, infra, for the particular bisphosphonate.

In certain embodiments, the dosage of a bisphosphonate administered to a human patient to treat colorectal cancer in accordance with the methods described herein is a dosage lower than the dosage approved by a regulatory agency (e.g., a dosage approved by the FDA) for any approved use. In a specific embodiment, the dosage of a bisphosphonate administered to a human patient to treat colorectal cancer in accordance with the methods described herein is a dosage lower the dosage provided in Table 2, infra, for the particular bisphosphonate.

In some embodiments, the frequency of administration of a dose of a bisphosphonate to a human patient to treat colorectal cancer is lower than the frequency approved by a regulatory agency (e.g., FDA) for any use. In specific embodiments, the frequency of administration of a bisphosphonate administered to a human patient to treat colorectal cancer in accordance with the methods described herein is a frequency lower than the frequency provided in Table 2, infra, for the particular bisphosphonate.

In certain embodiments, the dosage of a bisphosphonate administered to a human patient to treat colorectal cancer in accordance with the methods described herein is a dosage greater than the dosage approved by a regulatory agency (e.g., a dosage approved by the FDA) for any approved use. In a specific embodiment, the dosage of a bisphosphonate administered to a human patient to treat colorectal cancer in accordance with the methods described herein is a dosage greater than the dosage provided in Table 2, infra, for the particular bisphosphonate.

In some embodiments, the frequency of administration of a dose of a bisphosphonate to a human patient to treat colorectal cancer is a frequency greater than the frequency approved by a regulatory agency (e.g., the FDA) for any use. In specific embodiments, the frequency of administration of a bisphosphonate administered to a human patient to treat colorectal cancer in accordance with the methods described herein is a frequency greater than the frequency provided in Table 2, infra, for the particular bisphosphonate.

In a specific embodiment, the dosage of a bisphosphonate administered to a human subject to treat colorectal cancer in accordance with the methods described herein is a standard of care dosage. See Table 2, infra, for examples of standard care dosages for particular MEK inhibitors. In a specific embodiment, the dosage of a bisphosphonate administered to a human subject to treat colorectal cancer in accordance with the methods described herein is generally lower than the dosages that are administered as a standard of care dosage. In another specific embodiment, a dosage of the bisphosphonate administered to a human subject to treat colorectal cancer in accordance with the methods described herein is generally greater than the dosages that are administered in a standard of care dosage. In another specific embodiment, a dosage of the bisphosphonate administered to a human subject to treat colorectal cancer in accordance with the methods described herein is generally for longer periods of time than those described as a standard of care dosage. In some embodiments, the frequency of administration of a bisphosphonate ranges from once a day up to about once every eight weeks. In specific embodiments, the frequency of administration of the MEK inhibitor ranges from once a day, twice a day, once three times a day, every other day, once every three days, once a week, or once every other week. In certain embodiments, the frequency of administration of a bisphosphonate is once every 3 weeks, once a month, once every 2 months, once every 3 days, or every 6 months, or once a year. See Table 2, infra, for examples of the frequency of administration of particular bisphosphonates.

In some embodiments, the dosage of the bisphosphonate administered to a subject to treat colorectal cancer in accordance with the methods described herein is in the range of 0.01 to 50 mg/kg, of the subject's body weight. In one embodiment, the dosage of a bisphosphonate administered to a human subject to treat colorectal cancer in accordance with the methods described herein is in the range of about 0.1 mg/kg to about 1 mg/kg, about 0.1 mg/kg to about 1.5 mg/kg, about 0.1 mg/kg to about 2 mg/kg, about 0.1 mg/kg to about 2.5 mg/kg, about 0.1 mg/kg to about 3 mg/kg, about 0.1 mg/kg to about 3.5 mg/kg, about 0.1 mg/kg to about 4 mg/kg, about 0.1 mg/kg to about 4.5 mg/kg, about 0.1 mg/kg to about 5 mg/kg, about 0.1 mg/kg to about 5.5 mg/kg, about 0.1 mg/kg to about 6 mg/kg, about 0.1 mg/kg to about 6.5 mg/kg, about 0.1 mg/kg to about 7 mg/kg, about 0.1 mg/kg to about 7.5 mg/kg, about 0.1 mg/kg to about 8 mg/kg, about 0.1 mg/kg to about 8.5 mg/kg, about 0.1 mg/kg to about 9 mg/kg, about 0.1 mg/kg to about 9.5 mg/kg, about 0.1 mg/kg to about 10 mg/kg, about 0.1 mg/kg to 50 mg/kg, or about 1 mg/kg to 50 mg/kg, of the human subject's body weight. In another embodiment, the dosage of a bisphosphonate administered to a human subject to treat colorectal cancer in accordance with the methods described herein is in the range of about 0.1 mg/kg to 25 mg/kg, about 1 mg/kg to 25 mg/kg, or about 1 mg/kg to 10 mg/kg of the human subject's body weight.

In a specific embodiment, a bisphosphonate is administered to a human subject to treat colorectal cancer in accordance with the methods described herein is a dosage of 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg of the human subject's body weight.

In another specific embodiment, the dosage of a bisphosphonate administered to a subject to treat colorectal cancer in accordance with the methods described herein is a unit dose of 0.1 mg to 2000 mg. In specific embodiments, the dosage of a bisphosphonate is administered to a subject to treat colorectal cancer in accordance with the methods described herein is a unit dose of 0.1 mg to 1900 mg, 0.1 mg to 1800 mg, 0.1 mg to 1700 mg, 0.1 mg to 1600 mg, 0.1 mg to 1500 mg, 0.1 mg to 1400 mg, 0.1 mg to 1300 mg, 0.1 mg to 1200 mg, 0.1 mg to 1000 mg, 0.1 mg to 900 mg, 0.1 mg to 800 mg, 0.1 mg to 700 mg, 0.1 mg to 600 mg, 0.1 mg to 500 mg, 0.1 mg to 400 mg, 0.1 mg to 300 mg, 0.1 mg to 200 mg, 0.1 mg to 100 mg. In another specific embodiment, the dosage of a bisphosphonate administered to a subject to treat colorectal cancer in accordance with the methods described herein is a unit dose of 0.1 mg to 1600 mg, 1 mg to 1600 mg, 1 mg to 1500 mg, 1 mg to 1400 mg, 1 mg to 1300 mg, 1 mg to 1200 mg, 1 mg to 1100 mg or 1 mg to 1000 mg. In another specific embodiment, the dosage of a bisphosphonate administered to a subject to treat colorectal cancer in accordance with the methods described herein is a unit dose of 1 mg to 900 mg, 1 mg to 800 mg, 1 mg to 700 mg, 1 mg to 600 mg, 1 mg to 500 mg, 1 mg to 400 mg, 1 mg to 300 mg, 1 mg to 200 mg, 1 mg to 100 mg, or 1 mg to 50 mg. In a specific embodiment, the dosage of a bisphosphonate is administered is in the range of 0.01 to 10 g/m², and more typically, in the range of 0.1 g/m² to 7.5 g/m², of the subject's body weight. In one embodiment, the dosage administered to a human subject is in the range of 0.5 g/m² to 5 g/m², or 1 g/m² to 5 g/m² of the human subject's body's surface area.

In a specific embodiment, the dosage of a bisphosphonate used in accordance with the methods described is one described in the examples infra.

In certain embodiments, the dosage of a MEK inhibitor administered to a human subject in accordance with the methods described herein to treat colorectal cancer is an approved dosage for any indication and the dosage is altered depending on the condition of the subject (e.g., health and/or status of cancer). For example, the dosage of the MEK inhibitor administered to a human subject in accordance with the methods described herein to treat colorectal cancer may be reduced or the frequency of administering a dose may be reduced if the subject experiences an adverse reaction (e.g., a moderate or severe adverse reaction) as described in the examples below. In another example, the dosage of the MEK inhibitor may be increased if the subject does not experience an adverse reaction (e.g., a moderate or severe adverse reaction) associated with the inhibitor and the physician/clinician treating the subject believes that an increase in dosage may be beneficial to the subject. Similarly a physician/clinician may begin treating a human subject in accordance with the methods described herein with approved dosage of a bisphosphonate and reduce the dosage if the subject experiences an adverse reactions (e.g., a moderate or severe adverse reaction) to the bisphosphonate or physician/clinician may increase the dosage if the physician/clinician believes that the increase will be beneficial to the subject and the subject does not experience an adverse reaction (e.g., a moderate or severe adverse reaction) to the bisphosphonate. In treating the colorectal cancer patient, the physician/clinician may be monitoring the patient for adverse reaction to the MEK inhibitor and bisphosphonate and consider a course of treatment s/he believes appropriate given the condition of the patient (e.g., health and the stage of the patient's cancer). Examples of adverse reactions to bisphosphonates and MEK inhibitors are known in the art e.g., in the Physicians' Desk Reference or in prescribing information for the MEK inhibitor or bisphosphonate.

The MEK inhibitor or a composition thereof and the bisphosphonate or a composition thereof may be administered concurrently to the human subject to treat colorectal cancer in accordance with the methods described herein. The term “concurrently” is not limited to the administration of the MEK inhibitor or a composition thereof and the bisphosphonate or a composition thereof at exactly the same time, but rather, it is meant that they are administered to a human subject in a sequence and within a time interval such that they can act together. For example, the MEK inhibitor or a composition thereof and the bisphosphonate or a composition thereof may be administered at the same time or sequentially in any order at different points in time. For example, a first composition comprising a MEK inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) after the administration of a second composition comprising a bisphosphonate to a human subject in need thereof.

In various embodiments, the MEK inhibitor or a composition thereof and the bisphosphonate or a composition thereof are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the MEK inhibitor or a composition thereof and the bisphosphonate or a composition thereof are administered within the same office visit.

In a specific embodiment, a MEK inhibitor (e.g., trametinib) or a composition thereof is administered daily to a human subject to treat colorectal cancer and a bisphosphonate (e.g., zoledronic acid) or a composition thereof is administered every four weeks. The bisphosphonate may be administered intravenously and the trametinib may be administered orally. In a specific embodiment, the dosage, frequency and route of administration of a bisphosphonate and a MEK inhibitor are provided in the examples infra.

In some embodiments, a particular bisphosphonate and a particular MEK inhibitor are administered to a patient to treat colorectal cancer and after a certain period of time, the particular bisphosphonate, particular MEK inhibitor, or both are substituted with a different bisphosphonate, a different MEK inhibitor, or both, respectively. In certain embodiments, the certain period of time is about 1 week, 2 weeks, 3 weeks, 1 month, 2 months, 3 months, 4 months, 5 months, 6 months, 7 months, 8 months, 9 months, 10 months, 11 months, 12 months or longer. In some embodiments, the certain period of time is 1 to 3 weeks, 1 to 3 months, 3 to 6 months, 1 to 6 months, 6 to 9 months, 3 to 9 months, 9 to 12 months, or 6 to 12 months.

In certain embodiments, a MEK inhibitor or composition thereof and bisphosphonate or composition thereof are administered to treat the colorectal cancer patient as provided in their approved labels for any use. In some embodiments, the MEK inhibitor or composition thereof and the bisphosphonate are administered to the patient cyclically to treat colorectal cancer.

TABLE 1
List of MEK Inhibitors

| MEK | | | | | |
|---|---|---|---|---|---|
| In- | Manu- | | | Routes | |
| hibitor: | facture/ | Active | Inactive | of | |
| Drug | Dis- | Ingre- | Ingre- | Admin- | Dosing |
| Name | tributor | dients | dients | istration | Information |
| MEKI- | Novartis | Tra- | Table | Oral | Dosage |
| NIST ^{®} | Pharma- | metinib | core: | | forms: 0.5 |
| Tra- | ceutical | dimethyl | colloidal | | mg and 2 |
| metinib | Corp. | sulfoxide | silicon | | mg tablet. |
| (GSK1- | | | dioxide, | | Rec- |
| 120212) | | | croscar- | | ommended |
| | | | mellose | | as a |
| | | | sodium, | | single |
| | | | hypro- | | agent or |
| | | | mellose, | | in com- |
| | | | mag- | | bination |
| | | | nesium | | with |
| | | | stearate | | dabrafenib |
| | | | (vege- | | for the |
| | | | table | | treatment |
| | | | source), | | of un- |
| | | | mannitol, | | resectable |
| | | | micro- | | or |
| | | | crysta- | | metastatic |
| | | | lline | | melanoma |
| | | | cellulose, | | with |
| | | | sodium | | BRAF |
| | | | lauryl | | V600E |
| | | | sulfate. | | or V600K |
| | | | Coating: | | mutations; |
| | | | hypro- | | or in com- |
| | | | mellose, | | bination |
| | | | iron | | with |
| | | | oxide | | dabra- |
| | | | red | | fenib for |
| | | | (2 mg | | adjuvant |
| | | | tablets), | | treatment |
| | | | iron | | of |
| | | | oxide | | patients |
| | | | yellow | | with |
| | | | (0.5 mg | | melanoma |
| | | | tablets), | | with |
| | | | poly- | | BRAF |
| | | | ethylene | | V600E or |
| | | | glycol, | | V600K |
| | | | poly- | | mutations; |
| | | | sorbate | | and in com- |
| | | | 80 (2 mg | | bination |
| | | | tablets), | | with dabra- |
| | | | titanium | | fenib for |
| | | | dioxide. | | the treat- |
| | | | | | ment of |
| | | | | | patients |
| | | | | | with |
| | | | | | metastatic |
| | | | | | non-small |
| | | | | | lung cancer |
| | | | | | with BRAF |
| | | | | | V600E |
| | | | | | mutations. |
| | | | | | Recommended |
| | | | | | dosage is |
| | | | | | 2 mg orally |
| | | | | | once daily |
| | | | | | until disease |
| | | | | | progression |
| | | | | | or unacceptable |
| | | | | | toxicity. |
| | | | | | Recommended |
| | | | | | that 2 mg daily |
| | | | | | taken at least |
| | | | | | 1 hour before |
| | | | | | or at least 2 |
| | | | | | hours after meal. |
| | | | | | Dose reductions |
| | | | | | for adverse |
| | | | | | reactions |
| | | | | | includes a |
| | | | | | first dose |
| | | | | | reduction to |
| | | | | | 1.5 mg |
| | | | | | orally once daily |
| | | | | | and a second dose |
| | | | | | reduction to 1 mg |
| | | | | | orally once daily. |
| Selu- | Sponsor: | N/A | N/A | Oral | In clinical trials |
| metinib | Astra- | | | | for the treatment |
| (AZD- | Zeneca | | | | of non-small lung |
| 6244) | | | | | cancer. Dosages |
| | | | | | being tested |
| | | | | | include three |
| | | | | | 25 mg capsules |
| | | | | | administered |
| | | | | | orally, twice |
| | | | | | daily, (total |
| | | | | | dose 75 mg dose |
| | | | | | BID) on an |
| | | | | | uninterrupted |
| | | | | | schedule in |
| | | | | | combination with |
| | | | | | docetaxel. |
| MEK- | Array | bini- | Tablet | Oral | Dosage forms: |
| TOVI ^{®} | Bio- | metinib | core: | | 15 mg |
| Bini- | Pharma | | lactose | | tablets. |
| metinib | Inc. | | mono- | | Recommended |
| (MEK- | | | hydrate, | | dosage for |
| 162) | | | micro- | | treatment |
| | | | crysta- | | of patients with |
| | | | lline | | unresectable or |
| | | | cellu- | | metastatic |
| | | | lose, | | melanoma |
| | | | croscar- | | with a |
| | | | mellose | | BRAF V600E |
| | | | sodium, | | or V600K |
| | | | mag- | | mutation |
| | | | nesium | | is 45 mg |
| | | | stearate | | orally taken |
| | | | (vege- | | twice daily, |
| | | | table | | approximately 12 |
| | | | source), | | hours apart, in |
| | | | and | | combination with |
| | | | colloidal | | encorafenib until |
| | | | silicon | | disease |
| | | | dioxide. | | progression |
| | | | Tablet | | or unacceptable |
| | | | Coating: | | toxicity. |
| | | | poly- | | Dose |
| | | | vinyl | | reductions for |
| | | | alcohol, | | adverse reactions |
| | | | poly- | | includes a |
| | | | ethylene | | first dose |
| | | | glycol, | | reduction to 30 mg |
| | | | titanium | | orally twice daily |
| | | | dioxide, | | and a subsequent |
| | | | talc, | | modification to |
| | | | ferric | | permanently |
| | | | oxide | | discontinue if |
| | | | yellow, | | unable to |
| | | | ferro- | | tolerate 30 mg |
| | | | soferric | | orally twice daily |
| | | | oxide | | |
| COTE- | Genen- | cobi- | Tablet | Oral | Dosage forms: |
| LLIC ^{®} | tech | metinib | core: | | 20 mg tablets. |
| Cobi- | USA, | fumarate | micro- | | Recommended |
| metinib | Inc. | | crysta- | | dosage for |
| (XL518) | | | lline | | treatment of |
| | | | cellu- | | patients with |
| | | | lose, | | unresectable or |
| | | | lactose | | metastatic |
| | | | mono- | | melanoma |
| | | | hydrate, | | with a BRAF |
| | | | croscar- | | V600E or |
| | | | mellose | | V600K mutation |
| | | | sodium, | | in combination |
| | | | mag- | | with vere- |
| | | | nesium | | murafenib is 60 |
| | | | stearate. | | mg (three 20 mg |
| | | | Table | | tablets) orally |
| | | | Coating: | | taken once |
| | | | poly- | | daily for the |
| | | | vinyl | | first 21 days of |
| | | | alcohol, | | each 28-day cycle |
| | | | titanium | | until disease |
| | | | dioxide, | | progression or |
| | | | poly- | | unacceptable |
| | | | ethylene | | toxicity. |
| | | | glycol | | Dose reductions |
| | | | 3350, | | include a first dose |
| | | | talc. | | reduction to 40 mg |
| | | | | | orally once daily |
| | | | | | and second dose |
| | | | | | reduction to 20 mg |
| | | | | | orally once daily |
| | | | | | and a subsequent |
| | | | | | modification to |
| | | | | | permanently |
| | | | | | discontinue if |
| | | | | | unable to |
| | | | | | tolerate 20 mg |
| | | | | | orally once daily |
| Refa- | Bayer | Refa- | N/A | Oral | In clinical trials |
| metinib | | metinib | | | for treatment |
| (RDE- | | | | | of patients with |
| A119; | | | | | advanced or meta- |
| BAY | | | | | static cancer in |
| 869766) | | | | | combination with |
| | | | | | Regorafenib. The |
| | | | | | dose being tested |
| | | | | | is from 30 mg |
| | | | | | twice daily |
| | | | | | (b.i.d) or 20 mg |
| | | | | | b.i.d |
| Pima- | Merck | Pima- | N/A | Oral | Clinical trials for |
| sertib | KGaA | sertib | | | treating different |
| (AS70- | | | | | cancers in |
| 3026) | | | | | combination with |
| | | | | | other agents. |
| PD03- | Uni- | N/A | N/A | Oral | In clinical trials |
| 25901 | versity | | | | for treating |
| | of | | | | patients with |
| | Alabama | | | | neurofibromatosis |
| | at | | | | type-1 (NF1) and |
| | Birm- | | | | plexiform |
| | ingham | | | | neurofibromas. |
| | | | | | Dosages being |
| | | | | | tested include 2 |
| | | | | | mg/m2/dose by |
| | | | | | mouth on a |
| | | | | | bid with |
| | | | | | a maximum dose |
| | | | | | of 4 mg bid for 4 |
| | | | | | weeks. Patients |
| | | | | | receive drug on a |
| | | | | | 3 week on/1 week |
| | | | | | off schedule. |
| PD09- | Calbio- | N/A | N/A | Intra- | Dosages tested in |
| 8059 | chem | | | venous | mice for hepatoma |
| | | | | or | include 375 μM |
| | | | | Intra- | in 1 ml solution. |
| | | | | dermal | |
| AZD- | Astra- | N/A | N/A | Oral | Clinical trials for |
| 8330 | Zeneca | | | | treating patients |
| | | | | | with advanced |
| | | | | | malignancies. |
| | | | | | Dosages tested |
| | | | | | include 0.5 mg to |
| | | | | | 20 mg once-daily |
| | | | | | or twice-daily. |
| RO49- | Hoff- | N/A | N/A | Oral | Clinical trials for |
| 87655 | mann- | | | | treating patients |
| | La | | | | with advanced |
| | Roche | | | | and/or |
| | | | | | metastatic solid |
| | | | | | tumors. Dosages |
| | | | | | tested include |
| | | | | | 1 mg |
| | | | | | administered daily |
| | | | | | for 28 days until |
| | | | | | disease |
| | | | | | progression |
| | | | | | or toxicity. |
| RO51- | Memorial | N/A | N/A | Oral | In clinical trials |
| 26766 | Sloan | | | | for treating |
| (CH51- | Kettering | | | | patients with |
| 26766) | Cancer | | | | advanced KRAS- |
| | Center | | | | mutant lung |
| | | | | | cancer. Dosages |
| | | | | | being tested |
| | | | | | include 4 mg two |
| | | | | | times per week on |
| | | | | | days 1 and 4 of |
| | | | | | each week. The in- |
| | | | | | structions state |
| | | | | | that the drug |
| | | | | | should be taken |
| | | | | | by mouth on |
| | | | | | an empty stomach, |
| | | | | | either one hour |
| | | | | | before or two |
| | | | | | hours after a meal. |
| WX-554 | Wilex | N/A | N/A | Oral | Clinical trials for |
| | | | | | treating patients |
| | | | | | with solid tumors. |
| | | | | | Dosages tested |
| | | | | | include 150 mg |
| | | | | | once weekly |
| | | | | | or two doses |
| | | | | | at 75 mg twice |
| | | | | | weekly. |
| E6201 | Spirita | N/A | N/A | Intra- | In clinical trials for |
| | On- | | | venous | treating patients |
| | cology, | | | | with metastatic |
| | LLC | | | | melanoma |
| | | | | | central nervous |
| | | | | | system metastases |
| | | | | | (CNS). Dosages |
| | | | | | being tested |
| | | | | | include |
| | | | | | IV infusion |
| | | | | | administered at |
| | | | | | 320 mg/m² |
| | | | | | twice weekly on |
| | | | | | Days 1, 4, 8, 11, |
| | | | | | 15 and 18 for |
| | | | | | three weeks, |
| | | | | | and repeated |
| | | | | | every 28 days |
| | | | | | (1 cycle) until |
| | | | | | progression of |
| | | | | | disease, |
| | | | | | observation |
| | | | | | of unacceptable |
| | | | | | adverse events. |
| | | | | | Dose reductions |
| | | | | | for toxicity |
| | | | | | include a |
| | | | | | first dose |
| | | | | | reduction at 240 |
| | | | | | mg/m² twice |
| | | | | | weekly and 160 |
| | | | | | mg/m² twice |
| | | | | | weekly a |
| | | | | | second reduction |
| | | | | | administered over |
| | | | | | Days 1, 4, 8, |
| | | | | | 11, 15 and |
| | | | | | 18 for three |
| | | | | | weeks, repeated |
| | | | | | every 28 days. |
| GDC- | Genen- | N/A | N/A | Oral | Clinical trials for |
| 0623 | tech, | | | | treating patients |
| | Inc. | | | | locally advanced |
| | | | | | or metastatic solid |
| | | | | | tumors. Dosages |
| | | | | | tested include a |
| | | | | | QD regimen of |
| | | | | | 7-160 mg on a 21 |
| | | | | | day on/7 day off |
| | | | | | dosing schedule, |
| | | | | | and BID |
| | | | | | regimen of 45 |
| | | | | | mg on a 21-day |
| | | | | | on/7-day off |
| | | | | | dosing schedule. |
| CI-1040 | Pfizer | N/A | N/A | Oral | Clinical trials for |
| (PD18- | | | | | treating patients |
| 4352) | | | | | with advanced |
| | | | | | non-small-cell |
| | | | | | lung, breast, colon |
| | | | | | and pancreatic |
| | | | | | cancer. Dosages |
| | | | | | tested include 100 |
| | | | | | mg to 800 mg |
| | | | | | for 21 |
| | | | | | days repeated and |
| | | | | | every 28 days |
| | | | | | until disease |
| | | | | | progression |
| | | | | | or toxicity. |
| TAK- | Mill- | N/A | N/A | Oral | Clinical trials for |
| 733 | ennium | | | | treating patients |
| | Pharma- | | | | with advanced |
| | ceuticals, | | | | solid tumors. |
| | Inc. | | | | Dosages tested |
| | | | | | include 0.2-22 |
| | | | | | mg administered |
| | | | | | orally once |
| | | | | | daily on Days |
| | | | | | 1 through 21 in |
| | | | | | 28-day treatment |
| | | | | | cycle. |

TABLE 2
List of Bisphosphonates

| Bis- | | | | | |
|---|---|---|---|---|---|
| phos- | Manu- | | | Routes | |
| phates | facture/ | Active | | of | |
| Drug | Dis- | Ingre- | Inactive | Admin- | Dosing |
| Name | tributor | dients | Ingredients | istration | Information |
| Di- | Procter | Eti- | Tablet | Oral | Dosage forms: |
| dronel ^{®} | & | dronate | core: | | 400 mg |
| (eti- | Gamble | di- | Mag- | | tablet. |
| dronate) | Pharma- | sodium | nesium | | 1. Recommended |
| | ceuticals, | | stearate, | | dosage for |
| | Inc. | | micro- | | the treatment of |
| | | | crystalline | | symptomatic |
| | | | cellulose, | | Paget's disease |
| | | | and starch | | of bone is 5 to 10 |
| | | | | | mg/kg/day, not |
| | | | | | to exceed 6 |
| | | | | | months, or 11 to 20 |
| | | | | | mg/kg/day, not to |
| | | | | | exceed 3 months. |
| | | | | | 2. Recommended |
| | | | | | dosage for |
| | | | | | prevention and |
| | | | | | treatment |
| | | | | | of heterotopic |
| | | | | | ossification is: 20 |
| | | | | | mg/kg/day for 1 |
| | | | | | month before and |
| | | | | | 3 months after |
| | | | | | surgery (4 months |
| | | | | | total) for total |
| | | | | | hip replacement |
| | | | | | patients; and 20 |
| | | | | | mg/kg/day for 2 |
| | | | | | weeks followed by |
| | | | | | 10 mg/kg/day for |
| | | | | | 10 weeks for |
| | | | | | spinal cord injury. |
| Fosa- | Merck | Alen- | Tablet | Oral | Dosage forms: |
| max ^{®} | Sharp & | dronate | core: | | 5 mg, 10 mg, |
| (alen- | Dohme | sodium | Micro- | | 35 mg, 40 |
| dronate) | Corp., | | crystalline | | mg and 70 |
| | a sub- | | cellulose, | | mg tablets |
| | sidiary | | anhydrous | | and 70 mg |
| | of | | lactose, | | oral solution. |
| | Merck | | cros- | | 1. Recommended |
| | & Co., | | carmellose | | dosage for |
| | Inc. | | sodium, | | treatment of |
| | | | and mag- | | osteoporosis in |
| | | | nesium | | postmenopausal |
| | | | stearate | | women is one |
| | | | | | 70 mg tablet once |
| | | | | | weekly, or one |
| | | | | | bottle of 70 mg |
| | | | | | oral solution once |
| | | | | | weekly, or one 10 |
| | | | | | mg tablet once |
| | | | | | daily. |
| | | | | | 2. Recommended |
| | | | | | dosage for |
| | | | | | prevention of |
| | | | | | osteoporosis in |
| | | | | | postmenopausal |
| | | | | | women is one 35 |
| | | | | | mg tablet once |
| | | | | | weekly, or one |
| | | | | | 5 mg tablet |
| | | | | | once daily. |
| | | | | | 3. Recommended |
| | | | | | dosage for |
| | | | | | treatment to |
| | | | | | increase bone |
| | | | | | mass in men with |
| | | | | | osteoporosis is |
| | | | | | one 70 mg tablet |
| | | | | | once weekly, or |
| | | | | | one bottle of 70 |
| | | | | | mg oral solution |
| | | | | | once weekly, or |
| | | | | | one 10 mg |
| | | | | | tablet once daily |
| | | | | | 4. Recommended |
| | | | | | dosage for |
| | | | | | treatment of |
| | | | | | glucocorticoid- |
| | | | | | induced |
| | | | | | osteoporosis is |
| | | | | | one 5 mg tablet |
| | | | | | once daily, except |
| | | | | | for postmenopausal |
| | | | | | women not |
| | | | | | receiving estrogen, |
| | | | | | for whom the |
| | | | | | recommended |
| | | | | | dosage is one 10 |
| | | | | | mg tablet once |
| | | | | | daily. |
| | | | | | 5. Recommended |
| | | | | | dosage for treat- |
| | | | | | ment of Paget's |
| | | | | | disease of bone is |
| | | | | | 40 mg once a day |
| | | | | | for six months. |
| Acto- | Sanofi | Rise- | Cros- | Oral | Dosage forms: |
| nel ^{®} | | dronate | povidone, | | 5-mg tablet |
| (rise- | | sodium | ferric | | 1. Recommended |
| dronate) | | | oxide | | dosage for |
| | | | red | | treatment of |
| | | | (35-mg | | postmenopausal |
| | | | tablets | | osteoporosis |
| | | | only), | | is one 5-mg |
| | | | ferric | | tablet orally, |
| | | | oxide | | taken daily, or |
| | | | yellow (5 | | one 35-mg tablet |
| | | | and | | orally, taken |
| | | | 35-mg | | once a week. |
| | | | tablets | | 2. Recommended |
| | | | only), | | dose for the |
| | | | hydro- | | prevention of |
| | | | xypropyl | | postmenopausal |
| | | | cellulose, | | osteoporosis |
| | | | hydro- | | is one 5-mg |
| | | | xypropyl | | tablet orally, |
| | | | methyl- | | taken daily or |
| | | | cellulose, | | alternatively, one |
| | | | lactose | | 35-mg tablet |
| | | | mono- | | orally, taken |
| | | | hydrate, | | once a week. |
| | | | mag- | | 3. Recommended |
| | | | nesium | | dose for the |
| | | | stearate, | | treatment and |
| | | | micro- | | prevention of |
| | | | crystalline | | glucocorticoid- |
| | | | cellulose, | | induced |
| | | | poly- | | osteoporosis is |
| | | | ethylene | | one 5-mg |
| | | | glycol, | | tablet orally, |
| | | | silicon | | taken daily. |
| | | | dioxide, | | 4. Recommended |
| | | | titanium | | dose for the |
| | | | dioxide. | | Paget's Disease is |
| | | | | | 30 mg orally |
| | | | | | once daily |
| | | | | | for 2 months. |
| Boniva ^{®} | Genen- | Iban- | Tablet | Oral | Dosage forms: |
| (iban- | tech | dronate | core: | | 2.5 mg, or |
| dronate) | USA, | sodium | lactose | | 150 mg tablet. |
| | Inc. | | mono- | | Recommended |
| | | | hydrate, | | dosage for |
| | | | povidone, | | treatment and |
| | | | micro- | | prevention of |
| | | | crystalline | | postmenopausal |
| | | | cellulose, | | osteoporosis |
| | | | cros- | | is 2.5 mg |
| | | | povidone, | | once daily or |
| | | | purified | | 150 mg tablet |
| | | | stearic | | once a month. |
| | | | acid, | | |
| | | | colloidal | | |
| | | | silicon | | |
| | | | dioxide, | | |
| | | | and | | |
| | | | purified | | |
| | | | water. | | |
| | | | Tablet | | |
| | | | coating: | | |
| | | | hypro- | | |
| | | | mellose, | | |
| | | | titanium | | |
| | | | dioxide, | | |
| | | | talc, poly- | | |
| | | | ethylene | | |
| | | | glycol | | |
| | | | 6000, and | | |
| | | | purified | | |
| | | | water. | | |
| Boniva ^{®} | Genen- | Iban- | Sodium | | Dosage forms: |
| (iban- | tech | dronate | chloride, | | 3 mg/3 |
| dronate) | USA, | sodium | glacial | | mL solution. |
| | Inc. | | acetic acid, | | Recommended |
| | | | sodium | | dosage for |
| | | | acetate | | the treatment of |
| | | | and water | | postmenopausal |
| | | | | | osteoporosis is |
| | | | | | 3 mg every 3 |
| | | | | | months |
| | | | | | administered |
| | | | | | intravenously |
| | | | | | over a period |
| | | | | | of 15 to 30 |
| | | | | | seconds and |
| | | | | | must not be |
| | | | | | administered more |
| | | | | | frequently than |
| | | | | | once every |
| | | | | | 3 months. |
| | | | | | Injection must |
| | | | | | be administered |
| | | | | | intravenously |
| | | | | | only by a health |
| | | | | | care professional. |
| | | | | | Care must be |
| | | | | | taken not to |
| | | | | | administer intra- |
| | | | | | arterially or |
| | | | | | paravenously as |
| | | | | | this could lead to |
| | | | | | tissue damage |
| Reclast ^{®} | Novartis | Zole- | Mannitol | Intra- | Dosage forms: 5 |
| (zole- | Pharma- | dronic | and | venous | mg in a 100 |
| dronic | ceuticals | acid | sodium | | mL solution. |
| acid) | Corp. | mono- | citrate. | | 1. Recommended |
| | | hydrate | | | dosage for |
| | | | | | treatment of |
| | | | | | osteoporosis in |
| | | | | | postmenopausal |
| | | | | | women is a 5 |
| | | | | | mg infusion once a |
| | | | | | year given |
| | | | | | intravenously |
| | | | | | over no less than |
| | | | | | 15 minutes. |
| | | | | | 2. Recommended |
| | | | | | dosage for |
| | | | | | prevention of |
| | | | | | osteoporosis in |
| | | | | | postmenopausal |
| | | | | | women is a 5 |
| | | | | | mg infusion given |
| | | | | | once every 2 years |
| | | | | | intravenously over |
| | | | | | no less than |
| | | | | | 15 minutes. |
| | | | | | 3. Recommended |
| | | | | | dosage for osteo- |
| | | | | | porosis in men |
| | | | | | is a 5 mg infusion |
| | | | | | once a year given |
| | | | | | intravenously |
| | | | | | over no less than |
| | | | | | 15 minutes. |
| | | | | | 4. Recommended |
| | | | | | dosage for |
| | | | | | treatment and |
| | | | | | prevention of |
| | | | | | glucocorticoid- |
| | | | | | induced is a 5 |
| | | | | | mg infusion once |
| | | | | | a year given |
| | | | | | intravenously |
| | | | | | over no less than |
| | | | | | 15 minutes. |
| | | | | | 5. Recommended |
| | | | | | dosage for treat- |
| | | | | | ment of Paget's |
| | | | | | Disease of bone is |
| | | | | | a 5 mg infusion |
| | | | | | not be less than |
| | | | | | 15 minutes given |
| | | | | | over a constant |
| | | | | | infusion rate. |
| Zometa ^{®} | Novartis | Zole- | Mannitol, | Intra- | Dosage forms: 4 |
| (zole- | Pharma | dronic | USP, as | venous | mg/100 ml single- |
| dronic | Stein | acid | bulking | | use ready-to- |
| acid) | AG for | | agent, | | use bottle, and 4 |
| | Novartis | | water for | | mg/5 ml single- |
| | Pharma- | | injection, | | use vial of |
| | ceuticals | | and | | concentrate. |
| | Corp. | | sodium | | Recommended |
| | | | citrate, | | dosage for |
| | | | USP, as | | treatment of |
| | | | buffering | | patients with |
| | | | agent. | | multiple myeloma |
| | | | | | and metastatic |
| | | | | | bone lesions from |
| | | | | | solid tumors for |
| | | | | | patients with |
| | | | | | creatinine |
| | | | | | clearance (CrCl) |
| | | | | | greater than |
| | | | | | 60 mL/min is 4 |
| | | | | | mg infused over |
| | | | | | no less than 15 |
| | | | | | minutes every 3 to |
| | | | | | 4 weeks |
| | | | | | 2. Recommended |
| | | | | | dosage for |
| | | | | | treatment of |
| | | | | | patients for |
| | | | | | hypercalcemia of |
| | | | | | malignancy |
| | | | | | presenting with |
| | | | | | mild-to-moderate |
| | | | | | renal impairment |
| | | | | | prior to initiation |
| | | | | | of therapy (serum |
| | | | | | creatinine less |
| | | | | | than 400 μmol/L |
| | | | | | or less than |
| | | | | | 4.5 mg/dL). |
| Binosto ^{®} | Mission | Alen- | Mono- | Oral | Dosage forms: |
| (alen- | Pharma- | dronate | sodium | | 70 mg tablet |
| dronate | cal | sodium | citrate | | 1. Recommended |
| sodium) | Company | | anhydrous, | | dosage for |
| | | | citric acid | | treatment of |
| | | | anhydrous, | | osteoporosis in |
| | | | sodium | | postmenopausal |
| | | | hydrogen | | women is one 70 |
| | | | carbonate, | | mg effervescent |
| | | | and | | tablet once |
| | | | sodium | | weekly. |
| | | | carbonate | | 2. Recommended |
| | | | anhydrous | | dosage for |
| | | | as | | treatment to |
| | | | buffering | | increase bone |
| | | | agents, | | mass in men |
| | | | strawberry | | with osteoporosis |
| | | | flavor, | | is one 70 mg |
| | | | acesulfame | | effervescent tablet |
| | | | potassium, | | once weekly. |
| | | | and | | |
| | | | sucralose | | |
| CLAS- | Roche | Sodium | Tablet | Oral | Dosage forms: |
| TEON ^{®} | Products | clo- | core: | | 400 mg capsule. |
| (Clo- | Limited | dronate | Maize | | Recommended |
| dronate) | | | starch, | | dosage for |
| | | | talc, mag- | | treating osteolytic |
| | | | nesium | | lesions, |
| | | | stearate, | | hypercalcaemia |
| | | | sodium | | and bone pain |
| | | | starch | | associated with |
| | | | glycolate. | | skeletal metastases |
| | | | Tablet | | in patients |
| | | | coating: | | with carcinoma |
| | | | titanium | | of the breast |
| | | | dioxide | | or multiple |
| | | | (E171), | | myeloma is 4 |
| | | | indigotin | | capsules (1600 mg |
| | | | (E132)and | | sodium |
| | | | gelatin | | clodronate) daily. |
| SKEL- | Sanofi- | Tilu- | Sodium | Oral | Dosage forms: |
| LED ^{®} | aventis | dronate | lauryl | | 400 mg, tablet. |
| (Tilu- | U.S. | sodium | sulfate, | | Recommended |
| dronate) | LLC | | hydro- | | dosage treatment |
| | | | xypropyl | | of Paget's |
| | | | methyl- | | disease of bone |
| | | | cellulose | | (osteitis |
| | | | 2910, | | deformans) |
| | | | cros- | | is administered |
| | | | povidone, | | at 400-mg daily. |
| | | | magnesium | | |
| | | | stearate, | | |
| | | | and lactose | | |
| | | | mono- | | |
| | | | hydrate. | | |
| Aredia ^{®} | Novartis | Pami- | Mannitol, | Intra- | Dosage forms: |
| (Pami- | Pharma- | dronate | USP | venous | 30-mg, 60-mg, |
| dronate) | ceutical | di- | and | | and 90-mg vial |
| | Cor- | sodium | phosphoric | | solution |
| | poration | | acid | | 1. Recommended |
| | | | | | dosage for |
| | | | | | treating moderate |
| | | | | | hypercalcemia is |
| | | | | | 60 to 90 mg. The |
| | | | | | 60-mg dose is |
| | | | | | given as an initial, |
| | | | | | single dose, intra- |
| | | | | | venous infusion |
| | | | | | over at least 4 |
| | | | | | hours. The 90-mg |
| | | | | | dose must be |
| | | | | | given by an initial, |
| | | | | | single dose intra- |
| | | | | | venous infusion |
| | | | | | over 24 hours. |
| | | | | | 2. Recommended |
| | | | | | dosage |
| | | | | | for treating severe |
| | | | | | hypercalcemia is |
| | | | | | 90 mg given by |
| | | | | | an initial, single |
| | | | | | dose, intravenous |
| | | | | | infusion over |
| | | | | | 24 hours. |
| | | | | | 3. Recommended |
| | | | | | dosage for |
| | | | | | treating patients |
| | | | | | with moderate |
| | | | | | to severe |
| | | | | | Paget's disease of |
| | | | | | bone is 30 mg |
| | | | | | daily, administered |
| | | | | | as a 4- |
| | | | | | hour infusion |
| | | | | | on 3 consecutive |
| | | | | | days for a total |
| | | | | | dose of 90 mg. |
| | | | | | 4. Recommended |
| | | | | | dosage for |
| | | | | | treating patients |
| | | | | | with osteolytic |
| | | | | | bone lesions of |
| | | | | | multiple myeloma |
| | | | | | is 90 mg |
| | | | | | administered |
| | | | | | as a 4-hour |
| | | | | | infusion given on |
| | | | | | a monthly basis. |
| | | | | | 5. Recommended |
| | | | | | dosage for |
| | | | | | treating patients |
| | | | | | with osteolytic |
| | | | | | bone metastases |
| | | | | | is 90 mg ad- |
| | | | | | ministered over |
| | | | | | a 2-hour |
| | | | | | infusion given |
| | | | | | every 3-4 weeks |
| Neri- | Grü- | Neri- | N/A | Intra- | Cinical trials for |
| dronate | nenthal | dronic | | venous | treating patients |
| | GmbH | acid | | | with complex |
| | | | | | regional pain |
| | | | | | syndrome (CRPS). |
| | | | | | Dosages tested |
| | | | | | include 100 mg |
| | | | | | administered on |
| | | | | | Day 1, Day 4, Day |
| | | | | | 7, and Day 10, |
| | | | | | resulting in a total |
| | | | | | dose of neridronic |
| | | | | | acid 400 mg. |
| Olpa- | Eijkman | Olpa- | N/A | Intra- | Cinical trials |
| dronate | & | dronic | | venous | for treating |
| | Kuipers | acid | | | patients with |
| | Health- | | | | chronic back |
| | Care | | | | lower back pain. |
| | B.V. | | | | Dosage tested |
| | | | | | include 20 |
| | | | | | mg and 40 mg. |

In a specific embodiment, provided herein is a method for treating colorectal cancer, the method comprising administering to a human subject in need thereof first composition comprising trametinib and a second composition comprising zoledronic acid.

In some embodiments, provided herein is a method for treating colorectal cancer, the method comprising administering to a human subject in need thereof a first composition comprising trametinib dimethyl sulfoxide and a second composition comprising zoledronic acid.

In some embodiments, provided herein is a method for treating colorectal cancer, the method comprising administering to a human subject in need thereof two compositions, wherein one composition is MEKINIST® and the second composition comprises zoledronic acid.

In some embodiments, provided herein is a method for treating colorectal cancer, the method comprising administering to a human subject in need thereof two compositions, wherein one composition is cobimetinib and the second composition comprises zoledronic acid.

In some embodiments, provided herein is a method for treating colorectal cancer, the method comprising administering to a human subject in need thereof two compositions, wherein one composition comprises cobimetinib fumarate and the second composition comprises zoledronic acid.

In some embodiments, provided herein is a method for treating colorectal cancer, the method comprising administering to a human subject in need thereof two compositions, wherein one composition is COTELLIC® and the second composition comprises zoledronic acid.

In some embodiments, provided herein is a method for treating colorectal cancer, the method comprising administering to a human subject in need thereof two compositions, wherein one composition comprises binimetinib and the second composition comprises zoledronic acid.

In some embodiments, provided herein is a method for treating colorectal cancer, the method comprising administering to a human subject in need thereof two compositions, wherein one composition is MEKTOVI® and the second composition comprises zoledronic acid.

In some embodiments, provided herein is a method for treating colorectal cancer, the method comprising administering to a human subject in need thereof two compositions, wherein one composition comprises trametinib and the second composition is Zometa®.

In some embodiments, provided herein is a method for treating colorectal cancer, the method comprising administering to a human subject in need thereof two compositions, wherein one composition comprises trametinib dimethyl sulfoxide and the second composition is Zometa®.

In some embodiments, provided herein is a method for treating colorectal cancer, the method comprising administering to a human subject in need thereof two compositions, wherein one composition comprises MEKINIST® and the second composition is Zometa®.

In some embodiments, provided herein is a method for treating colorectal cancer, the method comprising administering to a human subject in need thereof a first composition comprising cobimetinib and a second composition is Zometa®.

In some embodiments, provided herein is a method for treating colorectal cancer, the method comprising administering to a human subject in need thereof two compositions, wherein one composition comprises cobimetinib fumarate and the second composition is Zometa®.

In some embodiments provided herein is a method for treating colorectal cancer, the method comprising administering to a human subject in need thereof COTELLIC® and Zometa®.

In some embodiments, provided herein is a method for treating colorectal cancer, the method comprising administering to a human subject in need thereof two compositions, wherein one composition comprises binimetinib and the second composition is Zometa®.

In some embodiments, provided herein is a method for treating colorectal cancer, the method comprising administering to a human subject in need thereof MEKTOVI® and Zometa®.

In some embodiments, provided herein is a method for treating colorectal cancer, the method comprising administering to a human subject in need thereof two compositions, wherein one composition is comprises trametinib and the second comprises ibandronate.

In some embodiments, provided herein is a method for treating colorectal cancer, the method comprising administering to a human subject in need thereof two compositions, wherein one composition comprises trametinib dimethyl sulfoxide and the second composition comprises ibandronate.

In some embodiments, provided herein is a method for treating colorectal cancer, the method comprising administering to a human subject in need thereof MEKINIST® and a composition comprising ibandronate.

In some embodiments, provided herein is a method for treating colorectal cancer, the method comprising administering to a human subject in need thereof two compositions, wherein one composition comprises cobimetinib and the second composition comprises a ibandronate.

In some embodiments, provided herein is a method for treating colorectal cancer, the method comprising administering to a human subject in need thereof two compositions, wherein one composition comprises cobimetinib fumarate and the second composition comprises ibandronate.

In some embodiments, provided herein is a method for treating colorectal cancer, the method comprising administering to a human subject in need thereof COTELLIC® and a composition comprising ibandronate.

In some embodiments, provided herein is a method for treating colorectal cancer, the method comprising administering to a human subject in need thereof two compositions, wherein one composition comprises binimetinib and the second composition comprises ibandronate.

In some embodiments, the method comprises administering to a human subject diagnosed with colorectal cancer MEKTOVI® and a composition comprising ibandronate.

In some embodiments, provided herein is a method for treating colorectal cancer, the method comprising administering to a human subject in need thereof BONIVA® and a composition comprising trametinib dimethyl sulfoxide.

In some embodiments, provided herein is a method for treating colorectal cancer, the method comprising administering to a human subject in need thereof MEKINIST® and BONIVA®.

In some embodiments, provided herein is a method for treating colorectal cancer, the method comprising administering to a human subject in need thereof BONIVA® and a composition comprising cobimetinib.

In some embodiments, provided herein is a method for treating colorectal cancer, the method comprising administering to a human subject in need thereof BONIVA® and a composition comprising cobimetinib fumarate.

In some embodiments, provided herein is a method for treating colorectal cancer, the method comprising administering to a human subject in need thereof COTELLIC® and BONIVA®.

In some embodiments, provided herein is a method for treating colorectal cancer, the method comprising administering to a human subject in need thereof BONIVA® and a composition comprising binimetinib.

In some embodiments, provided herein is a method for treating colorectal cancer, the method comprising administering to a human subject in need thereof MEKTOVI® and BONIVA®.

In some embodiments, a method of treating colorectal cancer as described herein results in one, two, three, or more of the following effects: complete response, partial response, objective response, increase in overall survival, increase in disease free survival, increase in objective response rate, increase in time to progression, stable disease, increase in progression-free survival, increase in time-to-treatment failure, and improvement or elimination of one or more symptoms of cancer. In a specific embodiment, a method of treating colorectal cancer as described herein results in an increase in overall survival. In another specific embodiment, a method of treating colorectal cancer as described herein results in an increase in progression-free survival. In another specific embodiment, a method of treating colorectal cancer as described herein results in an increase in overall survival and an increase in progression-free survival.

In a specific embodiment, “complete response” has the meaning understood by one of skill in the art. In a specific embodiment, a complete response refers to the disappearance of all signs of cancer in response to treatment. A complete response may not mean that the cancer is cured but that the patient is in remission. In a specific embodiment, colorectal cancer is in complete remission if clinically detectable disease is not detected by known techniques such as radiographic studies, bone marrow, and biopsy or protein measurements.

In a specific embodiment, “partial response” has the meaning understood by one of skill in the art. In a specific embodiment, a partial response refers to a decrease in the size of colorectal cancer in the human body in response to the treatment. In a specific embodiment, a partial response refers to at least about a 10%, 20%, 30%, 40%, 50%, 60%, 70%, 80%, or 90% decrease in all measurable tumor burden (e.g., the number of malignant cells present in the subject, or the measured bulk of tumor masses or the quantity of abnormal monoclonal protein) in the absence of new lesions.

In a specific embodiment, “overall survival” has the meaning understood by one of skill in the art. In a specific embodiment, overall survival refers to the length of time from either the date of the diagnosis or the start of treatment for colorectal cancer, that the human subject diagnosed with colorectal cancer is still alive. Demonstration of a statistically significant improvement in overall survival can be considered to be clinically significant if the toxicity profile is acceptable, and has often supported new drug approval.

Several endpoints are typically based on tumor assessments. These endpoints include disease free survival (“DFS”), objective response rate (“ORR”), time to progression (“TTP”), progression-free survival (“PFS”), and time-to-treatment failure (“TTF”). The collection and analysis of data on these time-dependent endpoints are often based on indirect assessments, calculations, and estimates (e.g., tumor measurements).

In a specific embodiment, “Disease Free Survival” (“DFS”) has the meaning understood by one of skill in the art. In a specific embodiment, disease-free survival refers to the length of time after primary treatment for colorectal cancer ends that the human subject survives without any signs or symptoms of cancer. DFS can be an important endpoint in situations where survival may be prolonged, making a survival endpoint impractical. DFS can be a surrogate for clinical benefit or it can provide direct evidence of clinical benefit. This determination is typically based on the magnitude of the effect, its risk-benefit relationship, and the disease setting. The definition of DFS can be complicated, particularly when deaths are noted without prior tumor progression documentation. These events may be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

In a specific embodiment, “objective response rate” (“ORR”) has the meaning understood by one of skill in the art. In one embodiment, an objective response rate is defined as the proportion of patients with tumor size reduction of a predefined amount and for a minimum time period. Response duration maybe measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST1.1 criteria) (see, e.g., Eisenhower et al., European J. Cancer 45: 228-247 (2009), which is hereby incorporated by reference in its entirety). The significance of ORR is assessed by its magnitude and duration, and the percentage of complete responses (no detectable evidence of tumor).

In a specific embodiment, “Time To Progression” (“TTP”) has the meaning understood by one of skill in the art. In a specific embodiment, time to progression refers to the length of time from the date of diagnosis or start of treatment for colorectal cancer until the cancer gets worse or spreads to other parts of the human body. In a specific embodiment, TTP is the time from randomization until objective tumor progression; TTP does not include deaths.

In a specific embodiment, “Progression Free Survival” (“PFS”) has the meaning understood by one of skill in the art. In a specific embodiment, PFS may refer to the length of time during and after treatment of colorectal cancer that the human patient lives with the cancer but it does not get worse. In a specific embodiment, PFS is defined as the time from randomization until objective tumor progression or death. PFS may include deaths and thus can be a better correlate to overall survival.

In a specific embodiment, “Time-to-Treatment Failure” (“TTF”) has the meaning understood by one of skill in the art. In a specific embodiment, TTF is composite endpoint measuring time from randomization to discontinuation of treatment for any reason, including disease progression, treatment toxicity, and death.

In a specific embodiment, stable disease refers to colorectal cancer that is neither decreasing or increasing in extent or severity.

In a specific embodiment, the RECIST 1.1 criteria is used to measure how well a human subject responds to the treatment methods described herein.

In a specific embodiment, the methods described herein may result in a decrease in tumor burden from baseline (e.g., 10%, 15%, 20%, 25%, 30%, 35%, 40%, 45%, 50%, 55%, or more, or 10% to 25%, 25% to 50%, or 25% to 75% decrease in tumor burden from baseline) and a partial response to treatment based on RECIST 1.1 criteria. In another specific embodiment, the methods of treatment described herein may result in a stable disease (e.g., stable decrease approximately for 1 month, 2 months, 3 months, 4 months, 5 months, 6 months, 7 months, 8 months, 9 months, 10 months, 11 months, 12 months, or more, or 2 to 6 months, 3 to 6 months, 3 to 9 months, 6 to 9 months, or 6 to 12 months). In another specific embodiment, the methods of treatment described herein result in one, two or more, or all the effects observed in the patient treated as described in Example 1, infra.

In some embodiments, the methods described herein may result in an improvement in and/or the elimination of one or more symptoms of colorectal cancer in the human subject. The one or more symptoms of colorectal cancer treated in accordance with the methods described herein may include, but are not limited to, changes in bowel habits, constipation, diarrhea, alternating diarrhea and constipation, rectal bleeding or blood in stool, abdominal bloating, abdominal cramps, abdominal discomfort, gas pains, feeling of incomplete bowel emptying, thinner than normal stools, unexplained weight loss, unexplained loss of appetite, nausea, vomiting, anemia, jaundice, weakness, and fatigue or tiredness. Colorectal cancer symptoms may also include pain, fracture, constipation, decreased alertness, shortness of breath, difficulty breathing, coughing, chest wall pain, extreme fatigue, increased abdominal girth, swelling of the feet and hands, yellowing or itch skin, bloating, swollen belly, pain, confusion, memory loss, headache, blurred or double vision, difficulty with speech, difficulty with movement, and seizures.

In a specific embodiment, the human patient treated in accordance with the methods described herein is one described, infra. In some embodiments, the colorectal cancer treated in accordance with the methods described herein is a colorectal adenocarcinoma, gastrointestinal stromal tumor, colorectal squamous cell carcinoma, gastrointestinal carcinoid tumor, primary colorectal lymphoma, colorectal melanoma, or colorectal leiomyosarcoma. In certain embodiments, the colorectal cancer treated in accordance with the methods described herein is an inherited form.

In some embodiments, the colorectal cancer treated in accordance with the methods described is an N-RAS mutant or H-RAS mutant. In a specific embodiment, the colorectal cancer treated in accordance with the methods described herein is KRAS-mutant colorectal cancer. In some embodiments, the colorectal cancer treated in accordance with the methods described herein contains a gene isoform (e.g., an oncogenic isoform(s) of HER1) previously demonstrated to activate one, two or all of the following: KRAS, HRAS or NRAS. In certain embodiments, the colorectal cancer treated in accordance with the methods described herein is KRAS-mutant colorectal adenocarcinoma cancer. In another specific embodiment, the colorectal cancer treated in accordance with the methods described herein has characteristics/features of a colorectal cancer described infra. In another embodiment, the colorectal cancer treated in accordance with the methods described herein is metastatic. Additional information regarding the colorectal cancer that may be treated in accordance with the methods described infra.

In a specific embodiment, a method of treating colorectal cancer described herein is the first line, second line, or third line of treatment the patient has undergone for colorectal cancer.

In a specific embodiment, the methods described herein are utilized in a combination with one or more other anti-cancer therapies, such as surgery, chemotherapy, radiation therapy, other kinase inhibitors and agents that block immune checkpoint inhibitors (e.g., anti-PDL1, anti-PD1, or anti-CTLA-4, antibodies). Examples of agents that block immune checkpoint inhibitors include, e.g., ipilimumab, nivolumad, pembrolizumab, atezolizumab, avelumab, durvalumab and cemiplimab. In a specific embodiment, a method for treating colorectal cancer comprises administering a first composition comprising a MEK inhibitor (e.g., trametinib), and a second composition comprising a bisphosphonate (e.g., zoledronic acid). The PDR describes currently available therapies for the treatment of cancer that may be used in combination with the methods described herein are known in the art as well as the dosages and frequency of use of such therapies (see, e.g., PDR 71^{st} 2017 Edition, which is hereby incorporated by reference in its entirety). In certain embodiments, one or more other anti-cancer therapies may be administered concurrently with, subsequent to, or prior to the combination of a MEK inhibitor or a composition thereof and a bisphosphonate or a composition thereof to treat colorectal cancer. For example, one, two or more other anti-cancer therapies may be administered to a human subject within minutes, hours, days, a week, 2 weeks, 3 weeks, 1 month, 2 months, 3 months, 4 months, 5 months, 6 months, or more when the human subject is being treated with a MEK inhibitor and a bisphosphonate in accordance with the methods described herein.

In some embodiments, no other anti-cancer therapies are administered to the human subject for colorectal cancer while the subject is being treated with a MEK inhibitor or a composition thereof and a bisphosphonate or a composition thereof as described herein.

In certain embodiments, one or more non-cancer therapies (e.g., pain reliever, antihistamine, or other anti-rash medications, such as, e.g., described infra) are administered to the human subject being treated for colorectal cancer in accordance with the methods described herein.

### Patient Population

The term “subject” and “patient” are used interchangeable herein to refer to a human subject. In one embodiment, a subject treated in accordance with the methods described herein has been diagnosed with colorectal cancer.

In a specific embodiment, a subject treated in accordance with the methods described herein may be unresponsive to approved therapies for colorectal cancer. In another specific embodiment, a subject treated in accordance with the methods described herein is refractory to one or more other anti-cancer therapies. In some embodiments, a human subject treated in accordance with the methods described herein has not undergone treatment with one or more other anti-cancer therapies.

In a specific embodiment, a subject to receive or a MEK inhibitor or a composition thereof and a bisphosphonate or a composition thereof has received other therapies to treat cancer. In another embodiment, the subject to receive or receiving a MEK inhibitor or a composition thereof and a bisphosphonate or a composition thereof has experienced one or more adverse effects or intolerance of one or more therapies to treat cancer. In another embodiment, the subject to receive or receiving a MEK inhibitor or a composition thereof and a bisphosphonate or a composition thereof has not experienced one or more adverse effects or intolerance of one or more therapies to treat cancer. In an alternative embodiment, the subject to receive or receiving a MEK inhibitor or a composition thereof and a bisphosphonate or a composition thereof has not received or is not receiving other therapies to treat cancer. In another embodiment, the subject to receive or receiving a MEK inhibitor or a composition thereof and a bisphosphonate or a composition thereof has been unresponsive to other therapies to treat cancer. In another embodiment, the subject to receive or receiving a MEK inhibitor or a composition thereof and a bisphosphonate or a composition thereof has had a relapse of colorectal cancer. In another embodiment, a subject treated in accordance with the methods described herein has or will undergo surgery to remove a tumor or neoplasm. The subject may receive a MEK inhibitor or a composition thereof and a bisphosphonate or a composition thereof before or after surgery. In some embodiments, a subject treated in accordance with the methods described herein has or will undergo radiation therapy, chemotherapy, or both. The subject may receive a MEK inhibitor or a composition thereof and a bisphosphonate or a composition thereof before or after having surgery, receiving radiation therapy, chemotherapy, an agent that blocks an immune checkpoint inhibitor (e.g., an anti-PD-1, an anti-PDL1, or an anti-CTLA-4 antibody) or any combination of the foregoing. In a specific embodiment, a subject treated in accordance with the methods described herein has been or is receiving one or more of the anti-cancer therapies described in the examples infra.

In certain embodiments, a MEK inhibitor or a composition thereof and a bisphosphonate or a composition thereof is administered to a subject as an alternative to chemotherapy, radiation therapy, hormonal therapy, targeted therapy, and/or biological therapy/immunotherapy where the therapy has proven or may prove too toxic, i.e., results in unacceptable or unbearable side effects, for the subject. In some embodiments, a MEK inhibitor or a composition thereof and a bisphosphonate or a composition thereof are administered to a subject that is susceptible to adverse reactions from other therapies. The subject may, e.g., have a suppressed immune system (e.g., post-operative patients, chemotherapy patients, and patients with immunodeficiency disease), have an impaired renal or liver function, be elderly, be a child, be an infant, have a neuropsychiatric disorder, take a psychotropic drug, have a history of seizures, or be on medication that would negatively interact with the therapies. In a specific embodiment, an elderly human is a human 65 years old or older.

In some embodiments, a subject treatment in accordance with the methods described herein may be in remission from colorectal cancer. In some embodiments, a subject treatment in accordance with the methods described herein may not be in remission from colorectal cancer. In some embodiment, the subject is not being treated with a bisphosphonate for an approved use, (e.g., loss of bone density, osteoporosis, osteitis deformans, and similar diseases).

In a specific embodiment, a subject being treated in accordance with the methods described herein is not being administered bisphosphonate to reduce the risk of cancer. In another specific embodiment, a subject being treated in accordance with the methods described herein is taking bisphosphonate for an approved use (e.g., to prevent or treat osteoporosis or similar disease). In another specific embodiment, a subject being treated in accordance with the methods described herein was but is no longer taking bisphosphonate for approved use or to reduce the risk of cancer. In another specific embodiment, a subject being treated with the methods described herein has never previously taken bisphosphonate for any use.

A subject treated in accordance with the methods described herein may have colorectal cancer that is a primary cancer or a metastatic cancer.

A subject treated in accordance with the methods described herein may have colorectal cancer caused by tumor cells that have metastasized, which may be a secondary or metastatic tumor. The cells of the tumor may be like those in the original tumor. In a specific embodiment, a subject treated in accordance with the methods described herein has metastatic colorectal cancer. In some embodiments, a subject treatment in accordance with the methods described herein may have KRAS-mutant colorectal cancer. In some embodiments, a subject treated in accordance with the methods described herein may have KRAS-mutant colorectal adenocarcinoma cancer. In certain embodiments, a subject treated in accordance with the methods described herein may have NRAS-mutant colorectal cancer or HRAS-mutant colorectal cancer. In some embodiments, a subject treated in accordance with the methods described herein may have colorectal cancer that contains a gene isoform previously demonstrated to activate one, two, or all of the following: KRAS, HRAS, or NRAS. In a specific embodiment, a subject treatment in accordance with the methods described herein may have colorectal adenocarcinoma. In a specific embodiment, a subject treated in accordance with the methods described herein may have gastrointestinal stromal tumor. In a specific embodiment, a subject treated in accordance with the methods described herein may have colorectal squamous cell carcinoma. In a specific embodiment, a subject treated in accordance with the methods described herein may have gastrointestinal carcinoid tumor. In a specific embodiment, a subject treated in accordance with the methods described herein may have primary colorectal lymphoma. In a specific embodiment, a subject treated in accordance with the methods described herein may have colorectal melanoma. In a specific embodiment, a subject treated in accordance with the methods described herein may have colorectal leiomyosarcoma. In a specific embodiment, a subject treated in accordance with the methods described herein has a colorectal cancer with characteristics/features of a colorectal cancer described infra. In another specific embodiment, a subject treated in accordance with the methods described herein is treated similar to a subject described in the examples infra.

In some embodiments, a human subject treated in accordance with the methods described herein may have various stages of colorectal cancer. In some embodiments, a human subject treated in accordance with the methods described herein may have Stage A colorectal cancer, which refers to when a tumor penetrates into the mucosa of the bowel wall but not further. In some embodiments, a human subject treated in accordance with the methods described herein may have Stage B colorectal cancer, which refers to when a tumor penetrates into and through the muscularis propria of the bowel wall. In some embodiments, a human subject treated in accordance with the methods described herein may have Stage C colorectal cancer, which refers to when a tumor penetrates into but not through muscularis propria of the bowel wall, there is pathologic evidence of colorectal cancer in the lymph nodes; or a tumor penetrates into and through the muscularis propria of the bowel wall, there is pathologic evidence of cancer in the lymph nodes. In some embodiments, a human subject treated in accordance with the methods described herein may have Stage D colorectal cancer, which refers to when a tumor has spread beyond the confines of the lymph nodes, into other organs, such as the liver, lung, or bone.

In certain embodiments, a subject treated in accordance with the methods described herein may have has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, a human subject treated in accordance with the methods described herein is 65 years old or older, or 70 years old or older. In some embodiments, a human subject treated in accordance with the methods described herein is 18 years of age or older. In a specific embodiment, a subject treated in accordance with the methods described herein is a subject such as described in the examples infra.

### Fly Avatars and Other Cancer Models

In some aspects, provided herein is a method for treating colorectal cancer, the method comprising administering to a human subject diagnosed with colorectal cancer a first composition comprising MEK inhibitor and a second composition comprising a bisphosphonate, wherein (a) cancer cells from the subject exhibit increased activity of one or more oncogenes and/or reduced activity of one or more tumor suppressors, and (b) the first composition comprising MEK inhibitor and the second composition comprising a bisphosphonate, when fed to a culture of a Drosophila larva avatar, allows the Drosophila larva avatar to survive to pupation, and wherein the Drosophila larva avatar is genetically modified such that upon induction through an external factor there is an increase in the activity of one or more orthologs of the subject's one or more oncogenes and/or inhibition of one or more orthologs of the human subject's one or more tumor suppressors in a larval tissue that is necessary for survival to pupation, which increase in activity and/or inhibition prevents an untreated Drosophila larva avatar from surviving to pupation. In a specific embodiment, the larval tissue that is necessary for survival is a hindgut or an imaginal disc. In another specific embodiment, the external factor is temperature.

In some embodiments, provided herein is a method for screening/selecting a specific MEK inhibitor or a composition thereof and a specific bisphosphonate or a composition thereof for treating a human subject diagnosed with colorectal cancer using a fly avatar of colorectal cancer, colorectal cancer cells or an animal model for colorectal cancer. In some embodiments, the specific MEK inhibitor or a composition thereof and the specific bisphosphonate or a composition thereof is used to treat colorectal cancer in accordance with the methods described herein increase survival of a fly avatar of colorectal cancer. In a specific embodiment, a fly avatar of colorectal cancer, such as described in International Patent Application Publication No. WO 2017/117344 A1 and U.S. Patent Application Publication No. 2019/0011435 A1 (each of which is incorporated herein by reference in its entirety) is used to identify the specific MEK inhibitor or a composition thereof and the specific bisphosphonate or a composition thereof that are used to treat colorectal cancer in accordance with the methods described herein. In another specific embodiment, a personalized fly avatar of colorectal cancer, which is generated such as described in Examples 1 and 3, infra, or in International Patent Application Publication No. WO 2017/117344 A1 and U.S. Patent Application Publication No. 2019/0011435 A1 (each of which is incorporated herein by reference in its entirety), is used to identify the specific MEK inhibitor or a composition thereof and the specific bisphosphonate or a composition thereof that are used to treat colorectal cancer in accordance with the methods described herein.

In some embodiments, provided herein is a method of treating colorectal cancer, the method comprising administering to a human subject in need thereof a specific MEK inhibitor or a composition thereof and a specific bisphosphonate or a composition thereof, wherein the specific MEK inhibitor or a composition thereof and the specific bisphosphonate or a composition thereof are identified in a fly avatar, such as described herein in U.S. Patent Application Publication No. 2019/0011435 A1 or International Patent Application Publication No. WO 2017/117344 A1, each of which is incorporated herein by reference in its entirety. In a particular embodiment, the specific MEK inhibitor or a composition thereof and the specific bisphosphonate or a composition thereof for use in treatment of colorectal cancer in accordance with the methods described herein results in an increased survival of a fly avatar of colorectal cancer, such as described herein, or in U.S. Patent Application Publication No. 2019/0011435 A1 or International Patent Application Publication No. WO 2017/117344 A1, each of which is incorporated herein by reference in its entirety.

In some embodiment, a fly avatar of colorectal cancer such as described herein, or in U.S. Patent Application Publication No. 2019/0011435 A1 or International Patent Application Publication No. WO 2017/117344 A1 (each of which is incorporated herein by reference in its entirety) is used to confirm a specific MEK inhibitor or a composition thereof and a specific bisphosphonate or a composition thereof used in accordance with the methods described herein for treating colorectal cancer.

In some embodiments, a Drosophila avatar of a human subject's colorectal cancer is engineered by genetically modifying a fly to correspondingly increase the activity of an ortholog(s) of the human subject's oncogene product(s), and inhibit the activity of an ortholog(s) of the human subject's tumor suppressor gene product(s) in a tissue/organ vital/necessary for survival (for example, the hindgut of the larva). In some embodiments, the activity of the engineered orthologs are designed to be under inducible control so that, e.g., upon induction, the untreated larva avatar (e.g., untreated Drosophila larva avatar) does not survive to pupation or mature to an adult fly. In some embodiments, this allows for the preferred activity to be controlled at will to facilitate screening.

In some embodiments, the combination of a specific MEK inhibitor or a composition thereof and a specific bisphosphonate or a composition thereof, for therapeutic efficacy are added to the food supplied to the culture of Drosophila avatars. Embryos are placed on the food; they begin consuming the food as larvae, at which point the activity of the transgenic orthologs are or have been induced. Therapeutic efficacy of the specific MEK inhibitor or a composition thereof and the specific bisphosphonate or a composition thereof is indicated by survival of the larva. In some embodiments, the assay does not require tumor visualization, expensive equipment, or detection of markers not compatible with high through-put screening for a readout. In some embodiments, a specific MEK inhibitor or a composition thereof and a specific bisphosphonate or a composition thereof arrived at or confirmed by the fly avatar assay may be communicated to the oncologist and ultimately to the patient for treatment. Where the MEK inhibitor and bisphosphonate involves combinations of known drugs where the toxicity and therapeutic indices are known, no further testing may be necessary.

In some embodiments, constructing a fly avatar, the following guiding principles should be followed: (a) The exact mutation of the colorectal cancer patient's tumor is not required to be engineered into the fly avatar. All that is required is to up-regulate the activity of orthologs of the patient's genes that demonstrate increased activity, and down-regulate the activity of orthologs of the patient's genes that exhibit decreased activity. (b) Due to the lethality of the engineered phenotype, the expression of the orthologs should be placed under inducible control so that the lethal activity can be induced at will, e.g., when the larva cultures are fed the combinations. (N.B., during fly development, embryos hatch to progress through three larval stages followed by pupation and metamorphosis to adult flies.) (c) While the activity of the orthologs is required to be altered in a larval organ vital to survival, the altered activity need not be confined/targeted solely to that organ: activity of the orthologs can also be altered in other tissues. In some embodiments, it is critical for the assay that the altered expression/activity occur in an organ vital to survival of the larvae in order to rapidly test a specific MEK inhibitor or a composition thereof and a specific bisphosphonate or a composition thereof for efficacy by incorporating into the larval food, and using survival of the larvae as the readout.

In some embodiments, to generate a fly avatar of colorectal cancer that reflects the patient's specific genomic complexity, fly orthologs are altered to identify a genomic analysis in the fly's hindgut using a GAL4/UAS expression system. In a specific embodiment, transgenes downstream of UAS (a yeast-derived promoter that is responsive specifically to the yeast GAL4 transcription factor) are cloned and transgenic flies containing a stable genomic insertion of UAS-transgenes with flies directing GAL4 expression in the fly hindgut are targeted.

In some embodiments, the fly avatar of colorectal cancer may be exposed to a specific MEK inhibitor or a composition thereof and a specific bisphosphonate or a composition thereof. In some embodiments, the fly avatar of colorectal cancer may be exposed to two or more specific MEK inhibitors or compositions thereof and specific bisphosphonates or compositions thereof at the same time, or sequentially. In some embodiments, the fly avatar of colorectal cancer may be exposed a first composition comprising a specific MEK inhibitor and a second composition comprising a specific bisphosphonate. In some embodiments, the fly avatar of colorectal cancer may be exposed to two or more specific MEK inhibitors or compositions thereof and specific bisphosphonates or compositions thereof at two or more overlapping time periods. In some embodiments, the fly avatar of colorectal cancer may be exposed to a first composition comprising MEK inhibitor for a first time period and exposed to a second composition comprising a bisphosphonate for a second period, wherein the first and second time periods at least partially overlap. In some embodiments, the fly avatar of colorectal cancer may be exposed to two or more specific MEK inhibitors or compositions thereof and specific bisphosphonates or compositions thereof at two or more non-overlapping time periods specific MEK inhibitors or compositions thereof and specific bisphosphonates or compositions thereof the fly avatar of colorectal cancer may be exposed to a first composition comprising MEK inhibitor for a first time period and exposed to a second composition comprising a bisphosphonate for a second period, wherein the first and second time periods do not overlap. As discussed herein, the exposure of the fly avatar of colorectal cancer to the specific MEK inhibitor or a composition thereof and the specific bisphosphonate or a composition thereof may be done by placing the combination in food.

In a specific embodiment, the screening assays for the specific MEK inhibitor or a composition thereof and the specific bisphosphonate or a composition thereof for treating colorectal cancer, in fly avatars of colorectal cancer is performed in individual tubes or wells of plate (e.g., a 96 well plate). The intent is to place food, the MEK inhibitors and bisphosphonates, and avatars into each tube or well. Food (e.g., fly, such as Drosophila, media) is placed into each tube or well; this may be done by hand or by automated sorting, e.g., via a liquid handler. In some embodiments, each specific MEK inhibitor or a composition thereof and specific bisphosphonate or a composition thereof may be added into duplicate tubes or wells at a chosen concentration that is not lethal to non-modified fly avatar of colorectal cancer. In some embodiment, the final food concentration of each of the specific MEK inhibitor or and specific bisphosphonate may be 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 125 μM, 150 μM, 175 μM, or 200 μM. In some embodiments, the specific MEK inhibitor and the specific bisphosphonate is mixed into the food and may be allowed to further diffuse for a period of time (e.g., 6-12 hours, 8-12 hours, 12-18 hours, 12-24 hours, 16-24 hours, 18 to 24 hours, 24 to 36 hours, or 12 to 36 hours). A designated number of transgenic fly embryos are placed into each tube or well on top of the solidified food/drug mixture. For example, each tube or well may contain 3, 4, 5, 6, 7, 8, 9, 10, 11, 12, 13, 14, 15 or more fly embryos. Each tube or plate may then be covered with a breathable substance and animals develop at the optimized temperature.

In some embodiments, a specific MEK inhibitor or a composition thereof and a specific bisphosphonate or a composition thereof may be determined to be effective if the presence of the specific MEK inhibitor and the specific bisphosphonate at least partly rescues the lethality caused by expression of the construct or expression system. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95% or more of fly avatars to adulthood. In some embodiments, the specific MEK inhibitor and the specific bisphosphonate may be determined to be effective if the presence of the specific MEK inhibitor and the specific bisphosphonate reduces the degree of lethality caused by expression of the construct or expression system. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95% or more of fly avatars to pupation. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95% or more of fly avatars to pupation. In some embodiments, the specific MEK inhibitor and the specific bisphosphonate may be determined to be effective if the presence of the specific MEK inhibitor and the specific bisphosphonate reduces the severity of a phenotype caused by expression of the construct or expression system.

In some embodiments, the specific MEK inhibitor and the specific bisphosphonate may be determined to be effective if the specific MEK inhibitor and the specific bisphosphonate causes the proteomic and/or phenomic profile of the avatar to more closely resemble the proteomic and/or phenomic profile of a healthy subject as compared to the proteomic and/or phenomic profile of the fly avatar of colorectal cancer in the absence of the specific MEK inhibitor and the specific bisphosphonate.

In some embodiments, the specific MEK inhibitor and the specific bisphosphonate may be determined to be effective if the presence of the specific MEK inhibitor and the specific bisphosphonate rescues the lethality caused by expression of the construct or expression system. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95%, or more of fly avatars to adulthood. In some embodiments, the specific MEK inhibitor and the specific bisphosphonate may be determined to be effective if the presence of the specific MEK inhibitor and the specific bisphosphonate reduces the degree of lethality caused by expression of the construct or expression system. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95%, or more of fly avatars to pupation. In some embodiments, the specific MEK inhibitor and the specific bisphosphonate may be determined to be effective if it rescues at least 0.5%, 1%, 5%, 10%, 15%, 20%, 25% 30%, 35%, 40%, 45%, 50%, 55%, 60%, 65%, 70%, 75%, 80%, 85%, 90%, 95%, or more of fly avatars to pupation. In some embodiments, the specific MEK inhibitor and the specific bisphosphonate may be determined to be effective the specific MEK inhibitor and the specific bisphosphonate causes a greater reduction in the degree of lethality in the avatar as compared to the reduction in the degree of lethality in the avatar caused by separate MEK inhibitors and bisphosphonates.

In some embodiments, provided herein is a method for screening/selecting for a specific MEK inhibitor or a composition thereof and a specific bisphosphonate or a composition thereof for treating a subject diagnosed with colorectal cancer, wherein cancer cells from the subject exhibit increased activity of one or more oncogenes and/or reduced activity of one or more tumor suppressors, comprises: screening a library of combination MEK inhibitors and/or bisphosphonates that when fed to a culture of a fly larva avatar, allow the fly larva avatar to survive to pupation, such that upon induction through an external factor there is an increase in the activity of an ortholog(s) of the subject's oncogene(s) and/or inhibition an ortholog(s) of the subject's tumor suppressor(s) in a larval tissue that is necessary for survival to pupation, which increase in activity and/or inhibition prevents an untreated fly larva avatar from surviving to pupation. In certain embodiments, the specific MEK inhibitor or a composition thereof and the specific bisphosphonate or a composition thereof allows 0.5%, 1%, 5%, 10%, 15%, 20%, 25% 30%, 35%, 40%, 45%, 50%, 55%, 60%, 65%, 70%, 75%, 80%, 85%, 90%, 95%, or more of fly larva avatar to survive to pupation. In certain embodiments, the specific MEK inhibitor or a composition thereof and the specific bisphosphonate or a composition thereof allows at least 0.5%, 1%, 5%, 10%, 15%, 20%, 25% 30%, 35%, 40%, 45%, 50%, 55%, 60%, 65%, 70%, 75%, 80%, 85%, 90%, 95%, or more of Drosophila larva avatar to survive to pupation. In certain embodiments, the specific MEK inhibitor or a composition thereof and the specific bisphosphonate or a composition thereof allows between 0.5% and 5%, between 5% and 15%, between 15% and 25%, between 25% and 35%, between 35% and 50%, between 50% and 70%, between 70% and 90%, or between 80% and 98% of fly larva avatar to survive to pupation.

In some embodiments, the fly avatar of colorectal cancer used in a screening assay described herein may be a personalized fly avatar of colorectal cancer. The personalized avatar may be used to screen for specific MEK inhibitors or compositions thereof and specific bisphosphonates or compositions thereof that may be effective to treat a colorectal cancer human subject.

In some embodiments, the fly avatar of colorectal cancer may be used to screen for specific MEK inhibitors or compositions thereof and specific bisphosphonates or compositions thereof for the treatment of colorectal cancer. In a specific embodiment, a fly avatar of colorectal cancer may be used in a screening assay described herein. In some embodiments, a fly avatar of colorectal cancer may be used test whether the human subject diagnosed with colorectal cancer will be responsive to a specific MEK inhibitor or a composition thereof and a specific bisphosphonate or a composition thereof. In a specific embodiment, provided herein is a fly avatar described in the examples infra.

In some embodiments, the fly avatar of colorectal cancer is a personalized avatar of colorectal cancer recapitulates a patient's genome, proteome, and/or phenome and can be used to select a specific MEK inhibitor or a composition thereof and a specific bisphosphonate or a composition thereof that may be effective for the treatment of colorectal cancer. In some embodiments, a colorectal cancer subject may be identified to comprise a mutation in KRAS gene. In some embodiments, a fly avatar of colorectal cancer may be engineered as described herein to contain a cDNA representing the ortholog of KRAS. Once the fly avatar of colorectal cancer is induced to express the cDNA, overexpression of the KRAS ortholog results. In some embodiments, the personalized fly avatar has the characteristics of a fly avatar described in the examples infra.

While the invention is not limited to the use of the fly avatar of colorectal cancer to select a specific MEK inhibitor or a composition thereof and a specific bisphosphonate or a composition thereof (other animal model avatars could be used for this purpose), the fly avatar of colorectal cancer model system as described herein offers the advantage of flexibility and speed—the genetic tools available for rapidly generating transgenic flies may be used to up- or down-regulate the activity of multiple orthologs of human gene products in the fly avatar to reflect the patient's profile. Moreover, the assay used to identify or test specific MEK inhibitors or a compositions thereof and specific bisphosphonates or a compositions thereof is rapid and does not require expensive equipment for read-outs. In some embodiments, colorectal cancer cells (e.g., colorectal cancer cell lines or colorectal cancer cells obtained from a human subject) are used to identify the MEK inhibitor and bisphosphonate to use in accordance with the methods described herein. In some embodiments, colorectal cancer cells (e.g., colorectal cancer cell lines or colorectal cancer cells obtained from a human subject) are used to confirm the a specific MEK inhibitor or a composition thereof and a specific bisphosphonate or a composition thereof to use in accordance with the methods described herein.

In certain embodiments, patient-derived xenografts in which colorectal cancer cells from a patient's colorectal cancer or a biopsy of a patient's colorectal cancer is implanted into an immunodeficient or humanized mouse, may be used to identify the MEK inhibitor and bisphosphonate to use in accordance with the methods described herein. In some embodiments, patient-derived xenograft may be used to confirm the a specific MEK inhibitor or a composition thereof and a specific bisphosphonate or a composition thereof to use in accordance with the methods described herein. In some embodiment, an animal model of colorectal cancer may be used to identify the specific MEK inhibitor and bisphosphonate to use in accordance with the method described herein.

In some embodiments, in accordance with the methods described herein, colorectal cancer cells from the human subject are analyzed to characterize the patient's mutations. In a specific embodiment, the colorectal cancer is KRAS-mutant colorectal cancer. In another specific embodiment, the colorectal cancer in accordance with the methods described herein is KRAS-mutant colorectal adenocarcinoma cancer. In some embodiments, the information is used to design and construct a Drosophila avatar that recapitulates the colorectal cancer patient's phenome. Similar information obtained from the colorectal cancer patients.

### Kits

In some aspects, provided herein are kits comprising a MEK inhibitor or a composition thereof described herein in one container and the bisphosphonate or a composition thereof described herein in another container. Examples of types of MEK inhibitors and types of bisphosphonates that may be included in a kit are disclosed infra. Examples of the types of compositions that may be included in the kits are also provided infra. In a specific embodiment, the compositions in the kits are sterile. In another specific embodiment, each container included in the kit is sterile. The kit may further comprise a label or printed instructions instructing the use of a MEK inhibitor or a composition thereof described herein and bisphosphonate or a composition thereof described herein for treatment of colorectal cancer.

In order that the invention disclosed herein may be more efficiently understood, examples are provided below. It should be understood that these examples are for illustrative purposes only and are not to be construed as limiting the invention in any manner.

### EXAMPLES

The examples below are intended to exemplify the practice of embodiments of the disclosure but are by no means intended to limit the scope thereof.

### Example 1—a Personalized Platform Identifies Trametinib Plus Zoledronate for Patient with KRAS Mutant Metastatic Colorectal Cancer

This example demonstrates the effectiveness of using the combination of trametinib and zoledronate to treat colorectal cancer. In addition, this example demonstrates the utility of a personalized fly avatar to identify drugs useful for treatment of colorectal cancer.

### Materials and Methods

Enrollment: The study was regulated by three separate protocols approved by the Mount Sinai Institutional Review Board (IRB): 1) a biorepository protocol that regulated inventory and processing of tumor and patient specific normal control (whole blood in EDTA) specimens; 2) a molecular analysis protocol that included genomic analysis, model building/validation and drug screening pipelines; and 3) a treatment protocol including a personalized treatment consent for the recommended therapy after the results are reviewed and approved a multidisciplinary tumor board.

Sample processing and genome assays: Genomic analysis was performed on (i) FFPE primary tumor specimen and (ii) whole blood collected at the time of consent to serve as a patient-matched normal control. Detailed protocols for sample processing, next generation sequencing assays, and data integration were described previously (Uzilov et al., “Development and Clinical Application of an Integrative Genomic Approach to Personalized Cancer Therapy,” Genome Med. 8:62 (2016), which is hereby incorporated by reference in its entirety).

Variant selection and validation: Whole exome sequencing of tumor and blood DNA identified 132 somatic and 965 rare germline variants. The analysis was focused on genes recurrently mutated in cancers including colorectal as well as those involved in cancer-relevant signaling pathways and cellular processes. To determine the likelihood that observed missense variants are deleterious (e.g., negatively impact protein function) two functional prediction algorithms were used: dbNSFP and CADD (Kircher et al., “A General Framework for Estimating the Relative Pathogenicity of Human Genetic Variants,” Nat. Genet. 46:310-315 (2014); Liu et al., “dbNSFP v3.0: A One-Stop Database of Functional Predictions and Annotations for Human Nonsynonymous and Splice-Site SNVs,” Hum. Mutat. 37:235-241 (2016), which are hereby incorporated by reference in their entirety). Variants predicted to be benign (e.g., unlikely to impact protein function) by both methods were eliminated. The remaining variants were first manually reviewed by examining the raw sequence reads to exclude false positives from automated WES variant calling algorithms. In addition, each variant was independently assessed by a Pacific Biosciences sequencing platform for orthogonal validation using targeted amplicon circular consensus sequencing as previously described (Uzilov et al., “Development and Clinical Application of an Integrative Genomic Approach to Personalized Cancer Therapy,” Genome Med. 8:62 (2016); Uzilov et al., “Identification of a Novel RASD1 Somatic Mutation in a USP8-mutated Corticotroph Adenoma,” Cold Spring Harb. Mol. Case Stud. 3, a001602 (2017), which are hereby incorporated by reference in their entirety). Using this method, it was confirmed that the presence of each variant except for SMARCA4, which was inconclusive strictly due to technical reasons and was included in the final selection of variants for building the Drosophila model.

Immunohistochemical Analysis: To confirm the findings of the gene-expression analysis, immunohistochemical assays were performed on 5 μm formalin-fixed, paraffin-embedded (FFPE) primary tumor sections for both FLT-1 (Abcam, catalog # ab9540, 1:200) and FLT-3 (Abcam catalog # ab150599, 1:100) with appropriate antibody controls (Donovan et al., “A Systems Pathology Model for Predicting Overall Survival in Patients with Refractory, Advanced Non-small-cell Lung Cancer Treated with Gefitinib,” Eur. J. Cancer 45:1518-1526 (2009), which is hereby incorporated by reference in its entirety). Immunohistochemical scoring was performed semi-quantitatively with an H-score (i.e., “histo” score) with intensity of staining ranging from 0-3+ multiplied by the percentage of positive expressing cells with a final score ranging from 0-300. The sample was considered over-expressed based on a discriminating threshold of >/= to an H-score of 150.

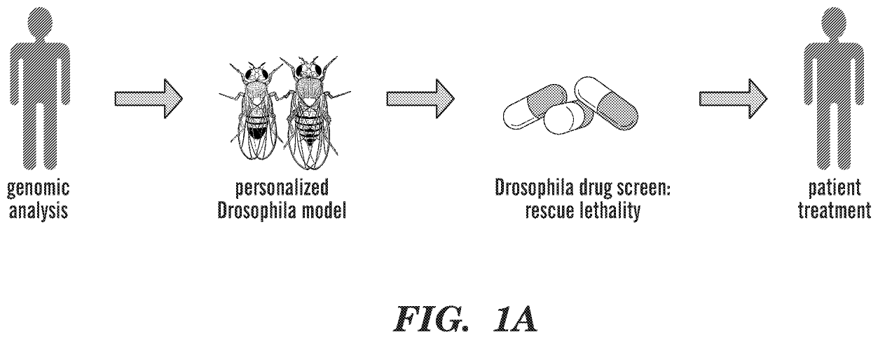

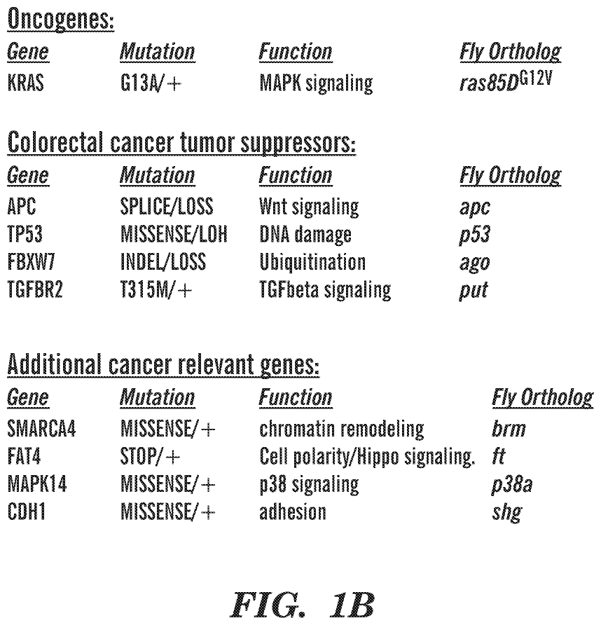

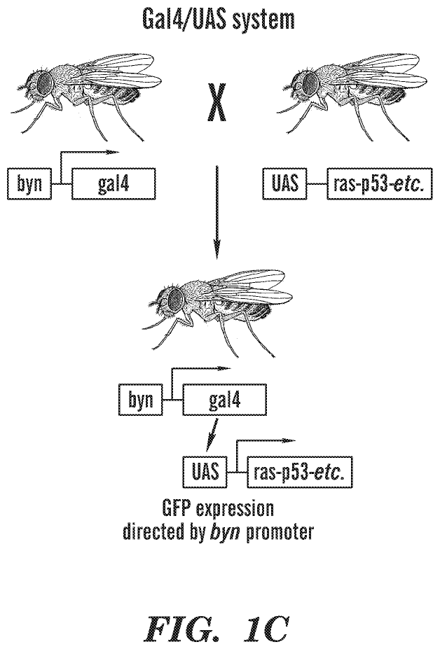

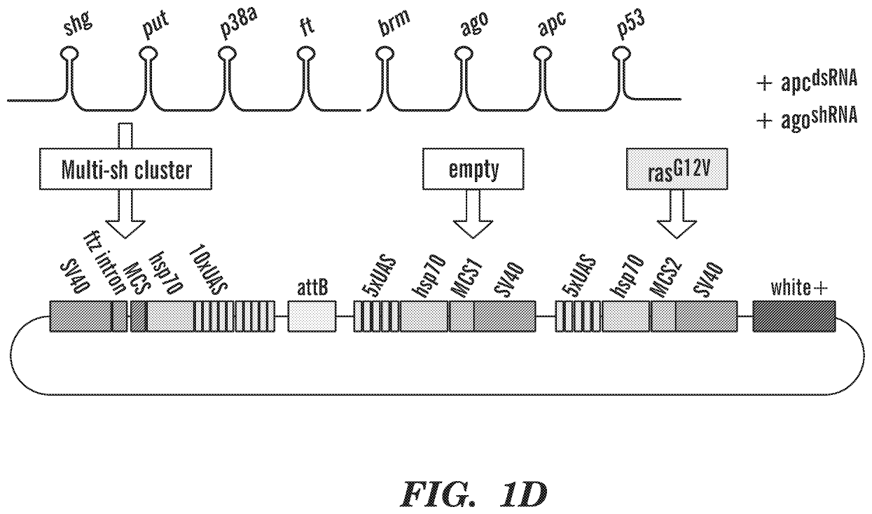

Model Building: Patient specific models were generated using a UAS-containing vector modified from a previously reported Drosophila transformation vector (Ni et al., “A Genome-scale shRNA Resource for Transgenic RNAi in Drosophila,” Nat. Methods 8:405-407 (2011), which is hereby incorporated by reference in its entirety). The modified vector contains three UAS cassettes each with their own UAS promoter, SV40 terminator sequences and unique multiple cloning sites (FIGS. 5 and 6). Oncogenic Drosophila ras85D(G12V) was PCR-amplified from a previously validated transgenic construct using primers designed to append restriction sites for enzymes FseI and PacI to the 5′ and 3′ ends of the product and cloned into one of the MCSs (FIG. 1D).

Short hairpins for gene knock-down were selected using DSIR, a publicly available tool for designing short hairpin RNAs (Vert et al., “An Accurate and Interpretable Model for siRNA Efficacy Prediction,” BMC Bioinformatics 7:520 (2006), which is hereby incorporated by reference in its entirety) following previously established hairpin selection criteria for Drosophila (Ni et al., “A Genome-scale shRNA Resource for Transgenic RNAi in Drosophila,” Nat. Methods 8:405-407 (2011), which is hereby incorporated by reference in its entirety). Individual hairpins were separated by spacer sequences found 5′ to well-expressed Drosophila microRNAs. To help ensure that a personalized model with a desired knock-down profile was obtained, two independent clusters that target the same 8 genes using different hairpin clusters were generated (006.1 and 006.2). Hairpin, spacer and final cluster sequences are provided in Tables 3A-3C. Full vector sequences and maps for both personalized constructs can be found in FIGS. 5 and 6.

Hairpin clusters were generated by gene synthesis (Genewiz). Sequence-confirmed products were then cloned into the multigenic vector using XbaI (5′) and NotI (3′). Transgenic flies were generated by PhiC31 mediated targeted integration into the attp40 site on the second chromosome (Bestgene) (Bischof et al., “An Optimized Transgenesis System for Drosophila Using Germ-line-specific phiC31 Integrases,” Proc. Nat'l. Acad. Sci. USA 104:3312-3317 (2007), which is hereby incorporated by reference in its entirety). To ensure strong knock-down for biallelically inactivated genes, previously validated transgenic RNAi knock-down lines for apc (VDRL) and ago (TRIP) were introduced by standard genetic crosses after transgenic flies were generated.

Model validation: Personalized models were validated by qPCR and western blots. Experimental and control animals for validation were generated by crossing both models (006.1 and 006.2) to a tub-gal4 tub-gal80^{ts} line to transiently and ubiquitously induce transgene expression for 3 days. Whole larvae with the genotypes 1) tub-gal4 tub-gal80^{ts}>UAS-006.1; UAS ago^{RNAi} UAS-apc^{RNAi}, 2) tub-gal4 tub-gal80^{ts}>UAS-006.2; UAS ago^{RNAi} UAS-apc^{RNAi} and 3) tub-gal4 tub-gal80^{ts}/+ as controls were collected (3 biological replicates/genotype; 6 larvae/replicate).

For protein extraction, larvae were homogenized using a motorized pestle in ice-cold 100 μl RIPA Buffer (Sigma) with Phosphatase Inhibitor Cocktail Set III (EMD Millipore) and Protease Inhibitor Cocktail (Roche). Lysates were centrifuged at 4° C. for 10 minutes at 13,000 RPM; supernatants (70 μl) were transferred to a fresh tube, 25 μl 4× NuPAGE LDS Sample Buffer and 10 μl NuPAGE 10× Reducing Agent (Invitrogen) were added. After a brief spin down, samples were boiled for 10 minutes, briefly spun down and centrifuged at 4° C. for 5 minutes at 13,000 RPM. 80 μl supernatant was transferred to new tubes and stored at −80° C. Western blots were performed (Bangi et al., “Cagan, Functional Exploration of Colorectal Cancer Genomes Using Drosophila,” Nat. Commun. 7:13615 (2016), which is hereby incorporated by reference in its entirety) using the following primary and secondary antibodies: Mouse anti-p53 (DSHB Dmp53-H3; 1:1000), mouse anti-dual phosphorylated ERK (dpERK, Sigma, 1:1000), mouse anti-Syntaxin as loading control (DSHB; 1:1000), goat-anti-mouse HRP secondary (1:10,000).

Larvae collected for RNA extraction were stored in 300 μl RNAlater (Life Technologies). RNA extraction was performed using the RNeasy plus kit with RNase-free DNase Set for on-column DNA digestion (Qiagen) following the manufacturer's instructions. RNA concentration was measured using Qubit. For qPCR analysis, 1 μg RNA was converted to cDNA using the High-capacity RNA-to-cDNA Kit (Life Technologies) and qPCR performed using PerfeCTa SYBR Green fastMix for IQ (VWR Scientific). A panel of 4 housekeeping genes (rp132, cyp33, gapdh and sdha) were first assayed to identify the best candidate and cyp33 was selected as providing the most robust and consistent results. qPCR data was analyzed using the double-ΔCT method (Sopko et al., “Combining Genetic Perturbations and Proteomics to Examine Kinase-phosphatase Networks in Drosophila Embryos,” Dev. Cell 31:114-127 (2014), which is hereby incorporated by reference in its entirety).

Model imaging: Whole guts were dissected from third instar byn-GAL4 tubulin-GAL80^{ts} UAS-GFP/UAS-transgene larvae that were induced at 25° C. for 4 days. Control and experimental animals were fixed with 4% paraformaldehyde, washed, and mounted. Images were taken at 5× (low magnification) and 10× in FIGS. 2A-2D. Quantification of the anterior portions of hindguts from drug treated animals were performed with ImageJ software using images captured at 10× magnification.

Drug Screening: Drugs in a custom Focused FDA library were purchased individually as powder from the following commercial sources: Selleck Chemicals, LC Laboratories, Tocris, and MedChemExpress. Drugs were dissolved in 100% DMSO or water based on the solubility information provided by the manufacturers. For each drug, the highest possible dose (based on solubility) that did not lead to detectable toxicity on wild type animals was selected for screening and drugs were aliquoted into 384-well plates.

The library was screened at a single dose for each drug along with DMSO controls (8 replicates/condition) by diluting each drug in the library 1:1000, which brings the DMSO concentration in the food to 0.1%. Drug-food mixtures were made using an automated liquid handling workstation (Perkin Elmer) by adding 0.7 μl drug into 12×75 mm round bottom test tubes (Sarstedt), followed by 700 μl semi-defined Drosophila medium (recipe obtained from the Bloomington Drosophila Stock Center) and mixing by pipetting.

After food was solidified, a mixture of experimental and control embryos (at a 1:2 ratio based on expected Mendelian ratios) were aliquoted into each drug/food tube (15 μl/tube). Embryo suspensions were generated using a buffer designed to minimize embryo clumping and settling (15% glycerol, 1% BSA, and 0.1% TWEEN 20 in water). Embryos for drug screening were generated from the following cross in cages: w/Y; UAS-006.1; UAS ago^{RNAi} UAS-apc^{RNAi}/Stub-gal80-T Xw UAS-dicer2; +; byn-gal4 UAS-GFP tub-gal80^{ts}/TM6, Hu, Tb. Embryos were obtained from each cage for 4-5 consecutive days by providing daily a fresh apple juice plate with yeast paste. Egg lays were performed at 22° C. to minimize transgene expression during embryogenesis to prevent embryonic defects or lethality that could not potentially be rescued by drug feeding. After embryos were aliquoted, drug tubes were transferred to 25° C. to induce transgene expression. After 2 weeks, the number of surviving experimental pupae (EP) were counted in each tube. Drugs that showed significantly higher numbers of experimental survivors compared to vehicle controls (multiple Student's t-tests corrected for multiple comparisons using the Holm-Sidak method, PRISM software) were considered hits.

Drug combination screens were performed by combining trametinib at its screening dose (1 μM in the food) with each drug in the library and mixing with Drosophila medium (8 replicates for each combination). DMSO and Trametinib alone served as controls. Drug combinations identified as candidate hits were re-tested in an independent experiment by combining the screening dose of trametinib with 3 different doses of each partner drug (original screening dose, 10% and 1% of screening dose). Experimental and control pupae (EP and CP, respectively) were counted for each tube and % survival to pupal stage calculated using the formula [(EP×2/CP)×100]. Statistical analysis was performed as described above.

### Results

Clinical Synopsis and Treatment History

A 53-year-old man without prior comorbidities was found to have a large partially obstructing mass of the distal sigmoid colon. A biopsy confirmed the diagnosis of colorectal adenocarcinoma. Intra-operatively, he was noted to have synchronous liver metastases. A laparoscopic lower anterior resection was performed with creation of a sigmoid end colostomy. Surgical pathology identified a moderately differentiated pT3N2a adenocarcinoma of the rectosigmoid colon with proficient DNA mismatch repair protein expression, lymphovascular and perineural invasion, and negative margins. A targeted next generation sequencing panel identified a KRAS(G13A) mutation; BRAF, NRAS and PIK3CA were wild type.

Six weeks after surgery, the patient initiated systemic therapy with FOLFOX and bevacizumab. Serum CEA, which was 9.6 ng/mL on the day of surgery, decreased to 7.1 ng/mL at the start of chemotherapy. After six months of therapy, his CEA normalized and a repeat computed tomography (CT) of the chest, abdomen, and pelvis showed a partial response by the liver metastases. He underwent a segment 8 hepatectomy, en bloc diaphragm resection, and colostomy reversal followed by three months of post-operative FOLFOX.

On a repeat CT one month later, multiple new lung nodules and left superior mediastinal adenopathy were identified. Serum CEA was normal at 1.8 ng/mL. The patient resumed chemotherapy with FOLFIRI and bevacizumab for an additional six months. Serial imaging performed during the chemotherapy regimen initially showed a slight decrease in size of the pulmonary nodules. Subsequent imaging 3 months later showed a mixed response: slight interval progression of some pulmonary nodules and stability in others. There was also an increase in scant subcentimeter retroperitoneal lymphadenopathy, and a more prominent left supraclavicular lymph node. A subsequent CT two months later revealed progression of lung metastases plus new left axillary, subpectoral and mediastinal adenopathy. Previously noted retroperitoneal and pelvic adenopathy had increased. Serum CEA was 2.3 ng/mL. Anticipating possible emergence of resistant disease, an experimental personalized treatment platform (FIG. 1A) was initiated while the patient received chemotherapy. Given the limited expected efficacy of available third line options upon failure of FOLFIRI/bevacizumab (Colucci et al., “Phase III Randomized Trial of FOLFIRI Versus FOLFOX4 in the Treatment of Advanced Colorectal Cancer: A Multicenter Study of the Gruppo Oncologico Dell'Italia Meridionale,” J. Clin. Oncol. 23:4866-4875 (2005); Deng et al., “Bevacizumab Plus Irinotecan, 5-fluorouracil, and Leucovorin (FOLFIRI) as the Second-line Therapy for Patients with Metastatic Colorectal Cancer, a Multicenter Study,” Med. Oncol. 30:752 (2013); Giantonio et al., “Bevacizumab in Combination with Oxaliplatin, Fluorouracil, and Leucovorin (FOLFOX4) for Previously Treated Metastatic Colorectal Cancer: Results from the Eastern Cooperative Oncology Group Study E3200,” J. Clin. Oncol. 25:1539-1544 (2007); Qu et al., “Value of Bevacizumab in Treatment of Colorectal Cancer: A Meta-analysis,” World J. Gastroenterol. 21:5072-5080 (2015); and Saltz et al., “Bevacizumab in Combination with Oxaliplatin-based Chemotherapy as First-line Therapy in Metastatic Colorectal Cancer: A Randomized Phase III Study,” J. Clin. Oncol. 26:2013-2019 (2008), which are hereby incorporated by reference in their entirety), the patient elected to enroll in the experimental study two months after the colostomy.

Genomic Analysis and Mutant Selection

As a first step towards developing a personalized Drosophila model, a comprehensive analysis of the patient's tumor genomic landscape was carried out (FIG. 1A). To this end, DNA from the primary tumor specimen and patient's blood (patient-specific normal control) was extracted and Whole Exome Sequencing (WES), targeted HotSpot panel, and Copy Number Analysis (CNA) assays were performed. The patient's tumor exhibited a large number of variants: 132 somatic and 965 rare germline variants.

To build a patient-specific Drosophila model, the analysis was focused on mutations in recurrently mutated cancer driver genes as well as genes that regulate cancer relevant signaling pathways and cellular processes. In addition to confirming the oncogenic KRAS(G13A) mutation, WES analysis of the patient's tumor showed biallelic loss of the well-established colorectal cancer drivers APC, TP53, and FBXW7 and a germline heterozygous missense mutation in TGFBR2 (FIG. 1B). Heterozygous somatic mutations in SMARCA4, FAT4, MAPK14, and a heterozygous germline mutation in CDH1 (FIG. 1B). While these genes are not frequently mutated in tumors, they regulate important cancer-relevant biological processes including chromatin remodeling, cell polarity and adhesion.

CNA identified a large number of alterations that included hundreds of genes. Using immunohistochemistry to assess gene expression levels, the analysis was focused on copy number alterations recurrently observed in colon tumors (N. Cancer Genome Atlas, “Comprehensive Molecular Characterization of Human Colon and Rectal Cancer,” Nature 487:330-337 (2012), which is hereby incorporated by reference in its entirety). The patient's tumor included a copy gain event in a region that encompassed receptor tyrosine kinases FLT1 and FLT3. However, immunohistochemistry analysis of the tumor specimen did not reveal an increase in the levels of either protein and they were not included in the Drosophila model.

Model Building and Validation

To build a Drosophila model that reflected the patient's specific genomic complexity, Drosophila orthologs of the nine genes identified in the genomic analysis were altered (FIG. 1B) in the fly's hindgut using the GAL4/UAS expression system (FIG. 1C) (Brand and Perrimon, “Targeted Gene Expression as a Means of Altering Cell Fates and Generating Dominant Phenotypes,” Development 118:401-415 (1993), which is hereby incorporated by reference in its entirety). Specifically, transgenes downstream of UAS, a yeast-derived promoter that is responsive specifically to the yeast GAL4 transcription factor, was cloned. To target transgenes to the hindgut, transgenic flies containing a stable genomic insertion of UAS-transgenes were crossed together with flies directing GAL4 expression in the hindgut (byn-GAL4; FIG. 1C). A UAS-GFP reporter was included to visualize transformed tissue.

A previously developed transformation vector (Ni et al., “A Genome-scale shRNA Resource for Transgenic RNAi in Drosophila,” Nat. Methods 8:405-407 (2011), which is hereby incorporated by reference in its entirety), was modified to contain three UAS cloning cassettes (FIG. 10). Oncogenic Drosophila ras85D(G12V) was placed under the control of one UAS promoter. To simultaneously reduce activity in eight tumor suppressors, synthetic clusters of sequences encoding short hairpin RNAs (shRNA) targeting each gene were generated; the sequences were modeled on endogenous miRNA clusters found in Drosophila and human genomes (Tables 3A-3C, see Methods). For genes biallelically inactivated in the patient—APC, TP53, and FBXW7—hairpins predicted to provide strong knockdown were selected; for the remaining genes with heterozygous variants, hairpins predicted to provide moderate knockdown were used. Hairpin sequences were assembled into a single oligonucleotide and placed under the control of a separate UAS promoter (FIG. 10, see Methods). Two stable transgenic Drosophila lines were generated to assess different hairpin predictions: 006.1 and 006.2 each with ras85D(G12V) but a different set of shRNA-based hairpin oligonucleotides targeting the same eight genes. After transgenic lines were established additional RNAi, constructs for apc and ago were introduced by standard genetic crosses to ensure strong knockdown. While both models showed effective knockdown of most target genes, model 006.1 showed a more favorable knockdown profile (FIG. 5A and FIG. 5B). Hindgut lysates were used to analyze knockdown of Shg and p53 proteins using commercially available antibodies; these results were consistent with qPCR data (FIG. 6A and FIG. 6B). Expression of the transgenes in the larval hindgut with the byn-GAL4 driver led to significant expansion of the anterior portion of the hindgut (FIGS. 2A-2B), reflecting aspects of transformation as were previously published (Bangi et al., “Cagan, Functional Exploration of Colorectal Cancer Genomes Using Drosophila,” Nat. Commun. 7:13615 (2016), which is hereby incorporated by reference in its entirety). Accordingly, the model 006.1 was selected for drug screening.

Drug Screening

It has previously been demonstrated that ‘rescue from lethality’ can be used as a quantitative phenotypic readout for high throughput drug screening. Targeting transgene expression to the developing hindgut epithelium can lead to broad transformation in the epithelium and organismal lethality; this lethality can be rescued by drugs mixed with the fly's food. A drug's ability to rescue Drosophila cancer models to pupation or adulthood indicates the drug is both effective and non-toxic.

For drug screening a custom “Focused FDA Library” was assembled consisting of 121 drugs FDA-approved for (i) cancer, (ii) non-cancer indications with reported anti-tumor effects, and (iii) non-cancer indications with cancer relevant targets. The first round of screening did not identify any drugs that provided significantly improved survival (Table 4A). This result is consistent with previous work demonstrating that genetically complex cancer models are often resistant to single agents and that drug combinations can be effective at addressing genetic complexity (Bangi et al., “Cagan, Functional Exploration of Colorectal Cancer Genomes Using Drosophila,” Nat. Commun. 7:13615 (2016), which is hereby incorporated by reference in its entirety).

Given the presence of oncogenic RAS in the patient's tumor, focus was placed on identifying effective drug combinations that included the MEK inhibitor trametinib. Trametinib is strongly effective against oncogenic RAS alone but not against highly multigenic colorectal cancer Drosophila models. A combination screen focusing on non-cancer drugs in a Focused FDA Library was screened in the presence of trametinib; the bisphosphonate class drug ibandronate was identified as strongly effective in combination with trametinib (Table 4B). These results were confirmed in an independent experiment in which trametinib was tested in combination with three different doses of ibandronate (FIG. 2C, Table 4C).

Bisphosphonates have been previously reported to have anti-tumor effects as single agents as well as in combination with different tyrosine kinase inhibitors. Two additional bisphosphonates, pamidronate and zoledronate, in combination with trametinib were tested. Zoledronate also proved to be an effective partner for trametinib (FIG. 2D, Table 4C). Ibandronate synergized with trametinib at a wider range of doses and provided a more significant rescue than zoledronate. The reason for this difference is not clear; for example, it may reflect subtle toxicity that was not apparent in controls, differences in off-target activities that lead to toxicity at higher doses, or differences in drug stability/metabolism in Drosophila. A multidisciplinary tumor board that included pharmacists, oncologists and clinical trial experts that reviewed the findings noted the oral ibandronate can cause esophagitis (Guay, “Ibandronate: A New Oral Bisphosphonate for Postmenopausal Osteoporosis,” Consult. Pharm. 20:1036-1055 (2005), which is hereby incorporated by reference in its entirety); intravenous (IV) administration of bisphosphonates would avoid esophagitis. Given the data supporting zoledronate as a potential anti-cancer agent (Konstantinopoulos et al., “Post-translational Modifications and Regulation of the RAS Superfamily of GTPases as Anticancer Targets,” Nature Reviews 6:541-555 (2007); Mo & Elson, “Studies of the Isoprenoid-mediated Inhibition of Mevalonate Synthesis Applied to Cancer Chemotherapy and Chemoprevention,” Exp. Biol. Med. (Maywood) 229:567-585 (2004); Wong et al., “HMG-CoA Reductase Inhibitors and the Malignant Cell: The Statin Family of Drugs as Triggers of Tumor-specific Apoptosis,” Leukemia 16:508-519 (2002); Yuen et al., “Bisphosphonates Inactivate Human EGFRs to Exert Antitumor Actions,” Proc. Nat'l. Acad. Sci. USA 111:17989-17994 (2014), which are hereby incorporated by reference in their entirety), the tumor board recommended a combination of IV zoledronate and oral trametinib for the patient.

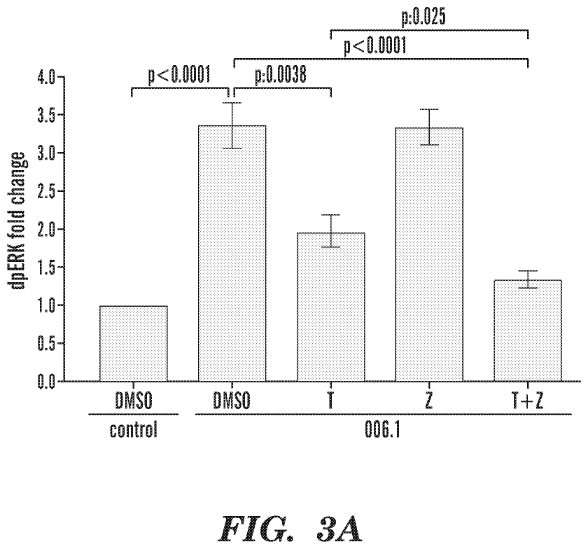

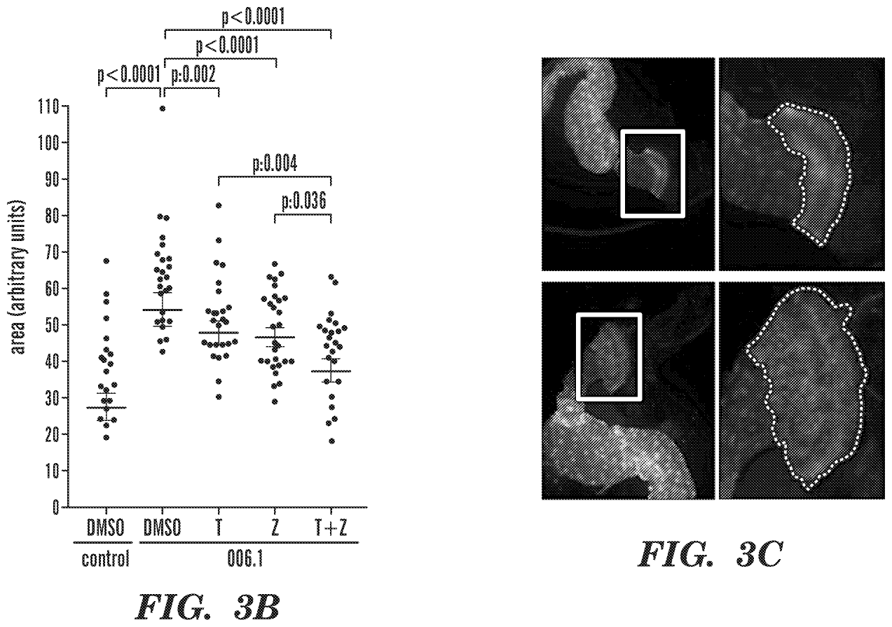

Drug response at the molecular and phenotypic level in the patient model's hindgut was explored (FIG. 3A). RAS/MAPK signaling pathway output using dually phosphorylated ERK (dpERK) in hindgut lysates from drug treated experimental animals was first evaluated. Lysates from the patient model demonstrated significantly increased dpERK levels compared to control animals (FIG. 3A, and FIG. 6C). Trametinib significantly reduced dpERK levels in the patient model while zoledronate had no detectable effect on MAPK signaling output. Combining trametinib with zoledronate led to a stronger reduction in dpERK levels than trametinib alone, indicating that zoledronate enhances the ability of trametinib to inhibit MAPK signaling. Regarding phenotypic changes, a statistically significant reduction in the expansion of the anterior portion of the hindgut in the patient model in response to each single agent was found; the trametinib/zoledronate combination directed a stronger rescue than either drug alone (FIG. 3B and FIG. 3C). Of note, the observation that zoledronate (i) partially rescued the anterior portion of the hindgut but (ii) had no effect on MAPK signaling output (FIG. 3A) suggests a complex, pleiotropic mechanism of action for the combination.

Patient Treatment

Prior to beginning third line therapy, the patient underwent an ophthalmologic exam and cardiac multigated acquisition (MUGA) scan, both of which were normal. A pre-treatment baseline CT reported target lesions including left axillary and paraaortic, aortocaval, and right external iliac adenopathy, and a left upper lobe pulmonary nodule. The sum of the longest diameter for all target lesions was 74 mm. Pre-treatment baseline CEA was 2.2 ng/mL.

Patient treatment was initiated with oral trametinib (2 mg daily) plus zoledronate (4 mg IV every 4 weeks). Within two weeks of starting therapy, the patient developed a grade 2 acneiform rash on his face, neck and upper back which was attributed to trametinib. The rashes progressed and the patient was prescribed minocycline, topical clindamycin and antihistamines. Despite these measures, the rash progressed to grade 3 in severity and the patient developed facial swelling without dyspnea or dysphonia by week four of therapy. Trametinib was suspended and he was referred to dermatology, confirming the diagnosis of drug-induced dermatitis. The patient's symptoms improved with the addition of prednisone. Zoledronate infusions continued every four weeks.

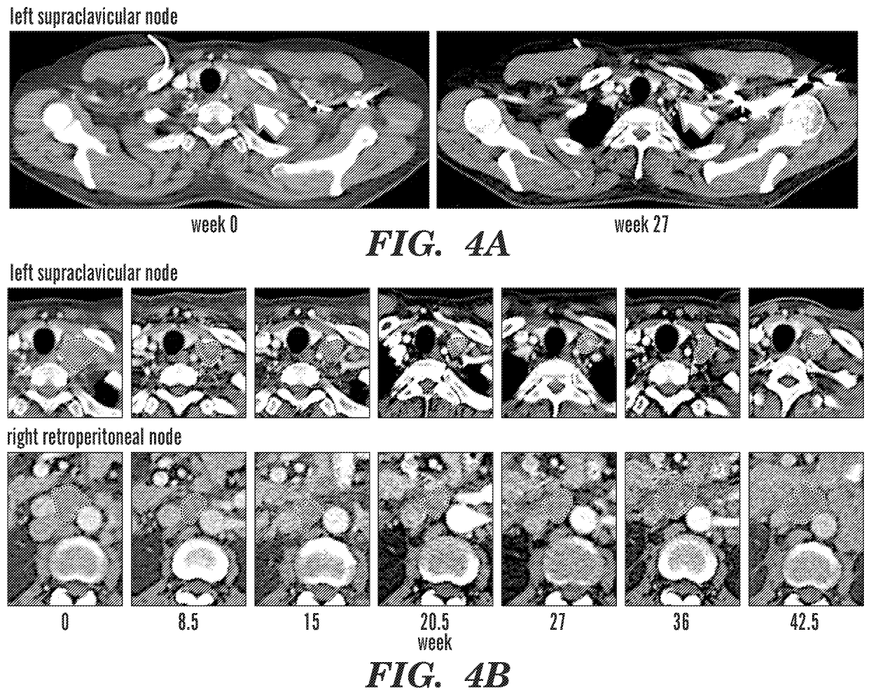

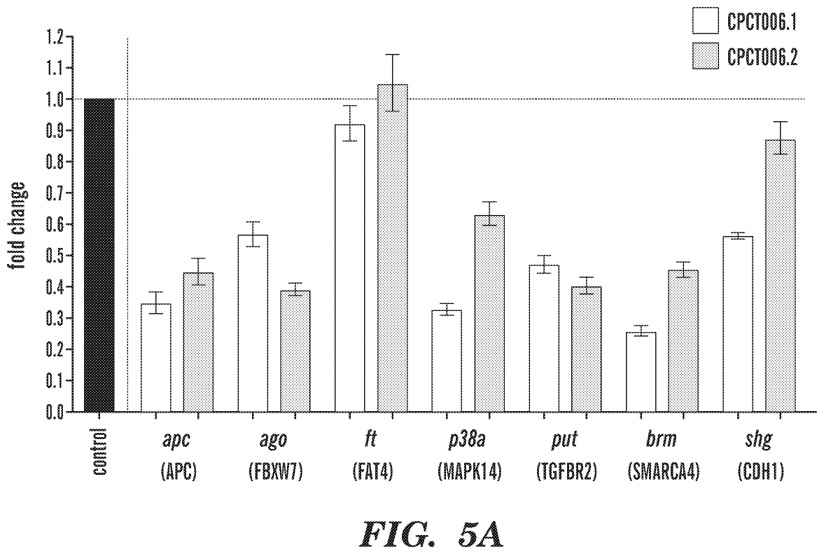

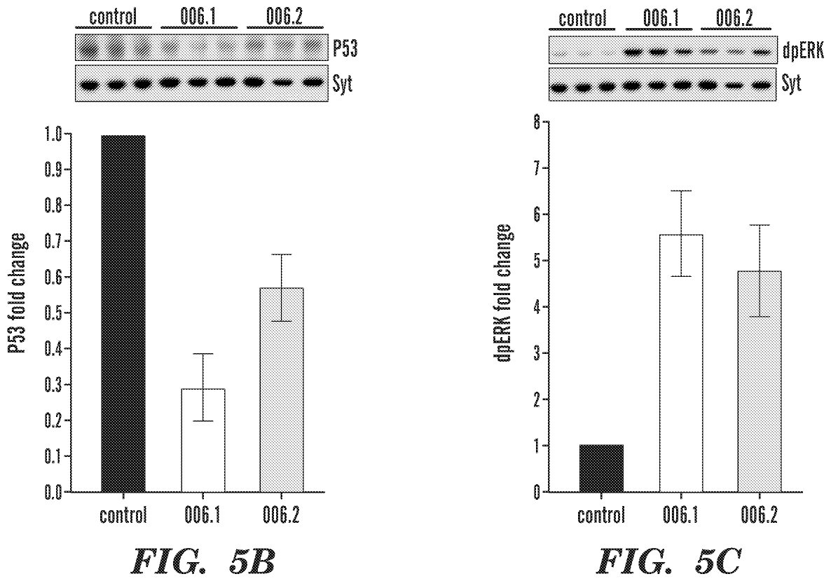

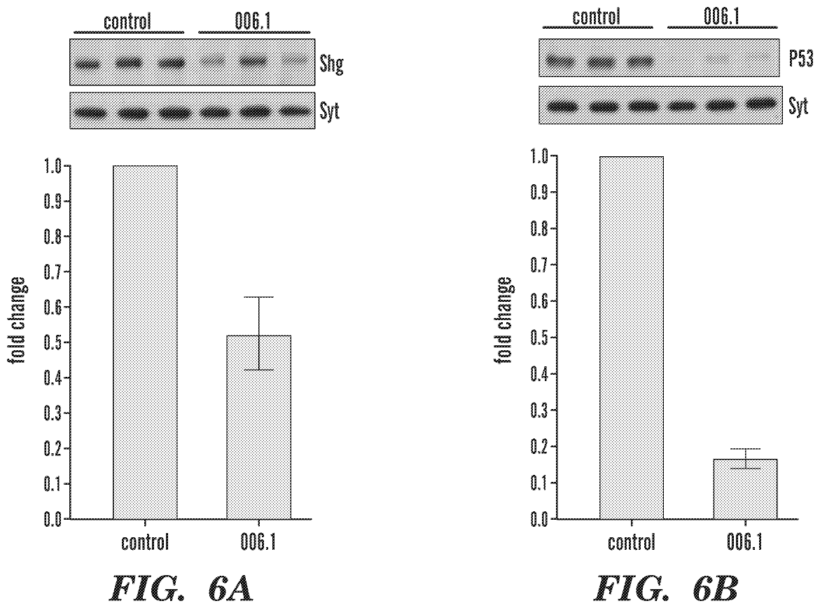

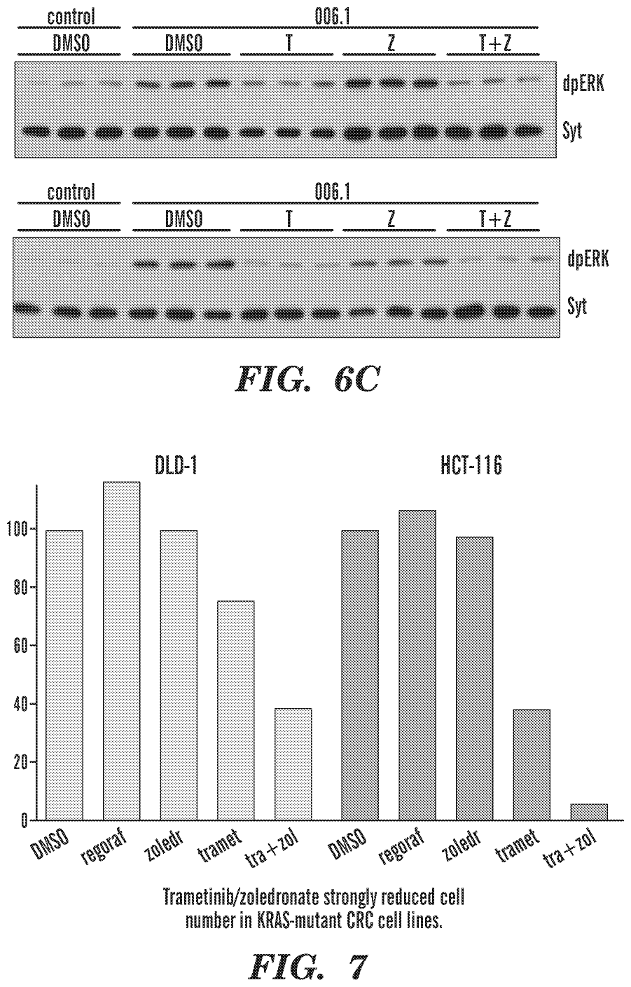

A CT of the chest abdomen and pelvis, performed eight weeks from the initial start date of therapy, revealed that the sum of the target lesion diameters had decreased to 41 mm, representing a 45% decrease from baseline and partial response to treatment based on RECIST 1.1 criteria (FIG. 4A and FIG. 4B, Table 5). The patient subsequently resumed trametinib a week later at a reduced dose of 0.5 mg every other day. Serum CEA at the time was 2.5 ng/ml. He tolerated the modified dose of trametinib well except for grade 1 pruritus. A repeat CT scan performed five weeks after resuming trametinib demonstrated a sustained partial response (PR) in target lesions (sum of diameters=41 mm). New peripancreatic and periportal adenopathy emerged measuring 16×15 mm and 15×53 mm, respectively. Based on these results, the dose of trametinib was increased to 0.5 mg daily. Twelve weeks after resuming trametinib, another CT was performed, showing a 10% increase in the sum of target lesions (now 45 mm) from nadir, but still 39% below baseline, indicative of a sustained partial response. The two new non-target lesions were also slightly larger (19×16 mm and 21×65 mm) but there were no new lesions.

Given the good tolerance of trametinib 0.5 mg daily without any new cutaneous toxicity, the dose was gradually increased to 1 mg daily. A further dose increase to trametinib 1.5 mg was attempted but the patient developed a pruritic rash after one week, causing the dose to be reduced back to 1 mg daily. A CT performed 18 weeks after resuming trametinib showed that the sum of target lesions was now 46 mm, constituting a 12% increase from nadir, but still 38% lower than baseline measurements. Additionally, the peripancreatic nodes had increased to 28×26 mm, and the periportal nodes to 27×85 mm.

Following the CT scan, trametinib was held while a ten-day course of stereotactic radiation was initiated to the abdominal adenopathy. Trametinib was resumed 11 days later at a dose of 1 mg daily. Serum CEA was 3.0 ng/ml. At this dose of trametinib, the patient occasionally experienced mild exacerbations of the drug rash and/or skin dryness involving his face or arms, but these reactions remained grade 1 in severity. Although the patient still maintained a good performance status (ECOG 1), he reported increasing fatigue, occasional postprandial nausea without vomiting, and abdominal bloating. He stopped trametinib on his own for four days due to these symptoms, then resumed. Approximately five weeks after completing radiation, a new CT demonstrated that the sum of the target lesions (now 62 mm) had increased by 51% from nadir, and the total sum was now 16% below baseline. New non-target lesions had also appeared: a left perirenal soft tissue nodule measuring 32×23 mm and an aortopulmonary window nodule measuring 15×18 mm. The irradiated periportal nodes were stable, but the peripancreatic nodes were slightly larger, measuring 28×26 mm. At this juncture, the decision was made to discontinue study therapy and switch to fourth line therapy with regorafenib.

Overall, the patient was treated with trametinib plus zoledronate for approximately eleven months, exhibiting a maximum 45% reduction in tumor burden. The primary toxicity was a severe rash controlled with antibiotics and antihistamines, permitting him to resume trametinib. The patient was eventually removed from treatment primarily due to emergence of novel lesions; the full genomic landscape of these lesions is unknown. There was an opportunity to explore the mutational profile of the treatment resistant peripancreatic and periportal nodes using a specimen obtained from an endoscopic ultrasound guided biopsy. The biopsy provided sufficient material for a targeted, high coverage analysis using Oncomine Comprehensive Panel version 2. No new mutations were reported on the panel, ruling out most druggable targets and at least many of the mutations known to promote resistance. A similar analysis using circulating cell-free DNA (cfDNA) identified a similar profile and also did not identify a specific resistance mechanism.

### Discussion

This example reports a novel treatment approach for a patient with advanced KRAS-mutant mCRC. Prior to the personalized therapy described herein, the patient had received but eventually failed multiple courses of chemotherapy. Anticipated response for this class of patients to third line targeted therapy or chemotherapy is poor with marginal improvement in overall survival (Grothey et al., “Regorafenib Monotherapy for Previously Treated Metastatic Colorectal Cancer (CORRECT): An International, Multicentre, Randomised, Placebo-controlled, Phase 3 Trial,” Lancet 381:303-312 (2013); Li et al., “Regorafenib Plus Best Supportive Care Versus Placebo Plus Best Supportive Care in Asian Patients with Previously Treated Metastatic Colorectal Cancer (CONCUR): A Randomised, Double-blind, Placebo-controlled, Phase 3 Trial,” Lancet. Oncol. 16:619-629 (2015); Mayer et al., “Randomized Trial of TAS-102 for Refractory Metastatic Colorectal Cancer,” N. Engl. J. Med. 372:1909-1919 (2015), which are hereby incorporated by reference in their entirety). Instead, based on extensive genomic analysis of the tumor we developed a ‘personalized’ Drosophila model as a whole animal screening platform was developed. A combination of trametinib plus a bisphosphonate reduced animal lethality. Treating the patient with trametinib/zoledronate led to a progression-free interval of three months overall, but a partial response of target lesions lasting eight months including a maximal 45% reduction in target lesions.

The model described here is one of the most genetically complex transgenic whole animal disease models described to date. Still, only a small subset of genomic alterations observed in the patient's tumor were able to be captured. Using functional prediction algorithms to prioritize those variants that are most likely to deleteriously impact protein function eliminated a significant number of variants most likely to be passenger events. Variants in genes identified as recurrently mutated drivers of cancer and those with clear cancer-relevant functions were focused on; however, the exclusion criteria are necessarily incomplete, and a large number of candidate variants remained. Further expanding the multigenic platform technology described here would provide an opportunity to generate even more sophisticated models that can better capture the genomic complexity of tumor genomic landscapes.

Most tumor genome landscapes contain a combination of heterozygous and homozygous loss of genes. Knockdown of a large number of genes to the desired level is a technically challenging issue. Use of hairpin sequences based on their predicted efficacy introduces a degree of uncertainty regarding how well they would perform in vivo, particularly in these genetically complex backgrounds. Generating two models each with a different set of hairpins targeting the same genes has been a useful approach to increase the likelihood of success. For instance, no significant knock down of ft in model 006.1 and ft or shg in model 006.2 were found. The knockdown profiles of the models would be further optimized by replacing the ineffective hairpins with improved version. However, building and validating additional models was not feasible in the time frame of the clinical study with the current approach.

Trametinib is a potent RAS pathway inhibitor, and its clinical failure to slow progression of most KRAS-mutant solid tumor types has been unexpected. This example demonstrates that trametinib can act on a nine-hit Drosophila model when dosed in combination with a bisphosphonate; this effectiveness translated into a partial response by the patient. The nature of zoledronate's synergy with trametinib is not clear. Zoledronate has been previously demonstrated to inhibit RAS pathway signaling through direct inhibition of EGFR activity and inhibition of prenylation (Konstantinopoulos et al., “Post-translational Modifications and Regulation of the RAS Superfamily of GTPases as Anticancer Targets,” Nature Reviews 6:541-555 (2007); Mo & Elson, “Studies of the Isoprenoid-mediated Inhibition of Mevalonate Synthesis Applied to Cancer Chemotherapy and Chemoprevention,” Exp. Biol. Med. (Maywood) 229:567-585 (2004); Wong et al., “HMG-CoA Reductase Inhibitors and the Malignant Cell: The Statin Family of Drugs as Triggers of Tumor-specific Apoptosis,” Leukemia 16:508-519 (2002); Yuen et al., “Bisphosphonates Inactivate Human EGFRs to Exert Antitumor Actions,” Proc. Nat'l. Acad. Sci. USA 111:17989-17994 (2014), which are hereby incorporated by reference in their entirety). Whether any of these activities are related to zoledronate's ability to synergize with trametinib is unclear.

Identifying an effective, unique drug combination—trametinib plus zoledronate—emphasizes the potential for moderately high-throughput screens that can be accomplished in a time frame that is useful for treating a patient. This approach may prove especially useful in tumors with challenging profiles, for example KRAS-mutant tumor types.

### Example 2—Effect of Zoledronate and Trametinib on Colorectal Cancer Cell Lines

RAS pathway inhibitors have shown limited efficacy in RAS-variant CRC patients. This includes trametinib, a potent and specific inhibitor of MEK. The Drosophila and (limited) patient data indicate that genetically complex RAS-variant colorectal tumors can be strongly sensitive to trametinib plus zoledronate.

In preliminary 2D culture experiments using human colorectal cancer (CRC) cell lines, it was found that zoledronate potentiated trametinib activity across a broad concentration curve to reduce expansion of two KRAS-variant human CRC cell lines—DLD1 and HCT116—when benchmarked against single drugs or against standard-of care regorafenib, FIG. 7 shows data at 15 nM.

### Example 3—Personalized Colorectal Cancer Fly Avatars Respond Strongly to Trametinib and Zoledronic Acid

Using similar techniques as described in Example 1, supra, three personalized fly avatars for three colorectal cancer patients were produced and the combination of zoledronic acid and trametinib was used to assess if the combination increased survival. These personalized fly avatars strongly responded to trametinib and zoledronic acid. Specifically, the fly avatars for three patients with the following features responded strongly to trametinib and zoledronic treatment, (1) Patient 1: KRAS, APC, TP53, SMAD2, ATM, PTEN, ARHGAP35, EP300, UPF1 mutants; (2) Patient 2: KRAS, APC, TP53, FBXW7, TGFβR2, SMARCA4, FAT4, MAPK14, CDH1; and (3) Patient 3: IGF2, TP53, PTEN, SMAD2, NCOR1, KMT2D, FANCL, LATS1, MUS81.

TABLE 3A
Hairpin sselected to target each gene,full hair pin sequences

| | | miR-1 | Variable | | Variable | miR-1 |
|---|---|---|---|---|---|---|
| | gene | flank | Passenger | Loop | Guide | flank 3′ |
| | name | 5′ (21 nt) | (21 nt) | (18 nt) | (21 nt) | (21 nt) |
| Cluster | generic | CCATATTCAG | NNNNNNNNNN | TAGTTATATT | NNNNNNNNNN | GCGAAATCTGGC |
| 006.1 | | CCTTTGAGAG | NNNNNNNNNN | CAAGCATA | NNNNNNNNNN | GAGACATCG |
| | | T | N | (SEQ ID | N | (SEQ ID |
| | | (SEQ ID | (SEQ ID | NO: 3) | (SEQ ID | NO: 5) |
| | | NO: 1) | NO: 2) | | NO: 4) | |
| | p53 | CCATATTCAG | CAACGTGGAC | TAGTTATATT | TTGAACTGAA | GCGAAATCTGGC |
| | | CCTTTGAGAG | GTTCAGTTCA | CAAGCATA | CGTCCACGTT | GAGACATCG |
| | | T | A | (SEQ ID | G | (SEQ ID |
| | | (SEQ ID | (SEQ ID | NO: 8) | (SEQ ID | NO: 10) |
| | | NO: 6) | NO: 7) | | NO: 9) | |
| | Apc | CCATATTCAG | CTCAAAGTTG | TAGTTATATT | TAAGAGTTGC | GCGAAATCTGGC |
| | | CCTTTGAGAG | TGCAACTCTT | CAAGCATA | ACAACTTTGA | GAGACATCG |
| | | T | A | (SEQ ID | G | (SEQ ID |
| | | (SEQ ID | (SEQ ID | NO: 13) | (SEQ ID | NO: 15) |
| | | NO: 11) | NO: 12) | | NO: 14) | |
| | ago | CCATATTCAG | TCCGATGACA | TAGTTATATT | TTTAAGTGTA | GCGAAATCTGGC |
| | | CCTTTGAGAG | ATACACTTAA | CAAGCATA | TTGTCATCGG | GAGACATCG |
| | | T | A | (SEQ ID | A | (SEQ ID |
| | | (SEQ ID | (SEQ ID | NO: 18) | (SEQ ID | NO: 20) |
| | | NO: 16) | NO: 17) | | NO: 19) | |
| | brm | CCATATTCAG | TACGACGAGG | TAGTTATATT | TAGAATGGTA | GCGAAATCTGGC |
| | | CCTTTGAGAG | ATACCATTCT | CAAGCATA | TCCTCGTCGT | GAGACATCG |
| | | T | A | (SEQ ID | A | (SEQ ID |
| | | (SEQ ID | (SEQ ID | NO: 23) | (SEQ ID | NO: 25) |
| | | NO: 21) | NO: 22) | | NO: 24) | |
| | ft | CCATATTCAG | CTGGCTAAGT | TAGTTATATT | TTTCTGTCCA | GCGAAATCTGGC |
| | | CCTTTGAGAG | GTGGACAGAA | CAAGCATA | CACTTAGCCA | GAGACATCG |
| | | T | A | (SEQ ID | G | (SEQ ID |
| | | (SEQ ID | (SEQ ID | NO: 28) | (SEQ ID | NO: 30) |
| | | NO: 26) | NO: 27) | | NO: 29) | |
| | p38a | CCATATTCAG | AAGGATGTAA | TAGTTATATT | TTGTGTTCAC | GCGAAATCTGGC |
| | | CCTTTGAGAG | AGTGAACACA | CAAGCATA | TTTACATCCT | GAGACATCG |
| | | T | A | (SEQ ID | T | (SEQ ID |
| | | (SEQ ID | (SEQ ID | NO: 33) | (SEQ ID | NO: 35) |
| | | NO: 31) | NO: 32) | | NO: 34) | |
| | put | CCATATTCAG | CTCACCGAGA | TAGTTATATT | TAGACTTGAA | GCGAAATCTGGC |
| | | CCTTTGAGAG | CTTCAAGTCT | CAAGCATA | GTCTCGGTGA | GAGACATCG |
| | | T | A | (SEQ ID | G | (SEQ ID |
| | | (SEQ ID | (SEQ ID | NO: 38) | (SEQ ID | NO: 40) |
| | | NO: 36) | NO: 37) | | NO: 39) | |
| | shg | CCATATTCAG | AAGAGTGCAA | TAGTTATATT | TTCTATTCTA | GCGAAATCTGGC |
| | | CCTTTGAGAG | ATAGAATAGA | CAAGCATA | TTTGCACTCT | GAGACATCG |
| | | T | A | (SEQ ID | T | (SEQ ID |
| | | (SEQ ID | (SEQ ID | NO: 43) | (SEQ ID | NO: 45) |
| | | NO: 41) | NO: 42) | | NO: 44) | |
| Cluster | p53 | CCATATTCAG | AGCGAGAACC | TAGTTATATT | TACACTGTTG | GCGAAATCTGGC |
| 006.2 | | CCTTTGAGAG | CAACAGTGTA | CAAGCATA | GGATTCTCGC | GAGACATCG |
| | | T | | (SEQ ID | T | (SEQ ID |
| | | (SEQ ID | (SEQ ID | NO: 48) | (SEQ ID | NO: 50) |
| | | NO: 46) | NO: 47) | | NO: 49) | |
| | Apc | CCATATTCAG | CTGGACGACC | TAGTTATATT | TCATCGAAGC | GCGAAATCTGGC |
| | | CCTTTGAGAG | AGCTTCGATG | CAAGCATA | TGGTCGTCCA | GAGACATCG |
| | | T | A | (SEQ ID | G | (SEQ ID |
| | | (SEQ ID | (SEQ ID | NO: 53) | (SEQ ID | NO: 55) |
| | | NO: 51) | NO: 52) | | NO: 54) | |
| | ago | CCATATTCAG | AAGCCTTTGT | TAGTTATATT | TTGACATAGA | GCGAAATCTGG |
| | | CCTTTGAGAG | ATCTATGTCA | CAAGCATA | TACAAAGGCT | CGAGACATCG |
| | | T | A | (SEQ ID | T | (SEQ |
| | | (SEQ ID | (SEQ ID | NO: 58) | (SEQ ID | ID NO: 60 |
| | | NO: 56) | NO: 57 | | NO: 59) | |
| | brm | CCATATTCAG | CTCGAAGCAT | TAGTTATATT | TTAAGGTGCT | GCGAAATCTGG |
| | | CCTTTGAGAG | CAGCACCTTA | CAAGCATA | GATGCTTCGA | CGAGACATCG |
| | | T | A | (SEQ ID | G | (SEQ ID |
| | | (SEQ ID | (SEQ ID | NO: 63) | (SEQ ID | NO: 65) |
| | | NO: 61) | NO: 62) | | NO: 64) | |
| | ft | CCATATTCAG | AGGTATGCGG | TAGTTATATT | TCGTAGTGAT | GCGAAATCTGG |
| | | CCTTTGAGAGT | GATCACTACG | CAAGCATA | CCCGCATACC | CGAGACATCG |
| | | (SEQ ID | A | (SEQ ID | T | (SEQ ID |
| | | NO: 66) | (SEQ ID | NO: 68) | (SEQ ID | NO: 70) |
| | | | NO: 67) | | NO: 69) | |
| | p38a | CCATATTCAGC | ATCGGTCTGC | TAGTTATATT | GAATATGTCC | GCGAAATCTGG |
| | | CTTTGAGAGT | TGGACATATT | CAAGCATA | AGCAGACCGA | CGAGACATCG |
| | | (SEQ ID | C | (SEQ ID | T | (SEQ ID |
| | | NO: 71) | (SEQ ID | NO: 73) | (SEQ ID | NO: 75) |
| | | | NO: 72) | | NO: 74) | |
| | put | CCATATTCAG | CACGGACATG | TAGTTATATT | TTGCATTCGT | GCGAAATCTGG |
| | | CCTTTGAGAG | CACGAATGCA | CAAGCATA | GCATGTCCGT | CGAGACATCG |
| | | T | A | (SEQ ID | G | (SEQ ID |
| | | (SEQ ID | (SEQ ID | NO: 78) | (SEQ ID | NO: 80) |
| | | NO: 76) | NO: 77) | | NO: 79) | |
| | shg | CCATATTCAG | TGTCCAGAAG | TAGTTATATT | TGCAGTGGTA | GCGAAATCTGG |
| | | CCTTTGAGAG | CTACCACTGC | CAAGCATA | GCTTCTGGAC | CGAGACATCG |
| | | T | A | (SEQ ID | A | (SEQ ID |
| | | (SEQ ID | (SEQ ID | NO: 83) | (SEQ ID | NO: 85) |
| | | NO: 81) | NO: 82) | | NO: 84) | |

gene
name

| | | |
|---|---|---|
| Cluster | p53 | CCATATTCAGCCTTTGAGAGTCAACGTGGACGTTCAGTTCAATAGTTATATTCAAG |
| 006.1 | | CATATTGAACTGAACGTCCACGTTGGCGAAATCTGGCGAGACATCG |
| | | (SEQ ID NO: 86) |
| | Apc | CCATATTCAGCCTTTGAGAGTCTCAAAGTTGTGCAACTCTTATAGTTATATTCAAG |
| | | CATATAAGAGTTGCACAACTTTGAGGCGAAATCTGGCGAGACATCG |
| | | (SEQ ID NO: 87) |
| | ago | CCATATTCAGCCTTTGAGAGTTCCGATGACAATACACTTAAATAGTTATATTCAAGC |
| | | ATATTTAAGTGTATTGTCATCGGAGCGAAATCTGGCGAGACATCG |
| | | (SEQ ID NO: 88) |
| | brm | CCATATTCAGCCTTTGAGAGTTACGACGAGGATACCATTCTATAGTTATATTCAAGC |
| | | ATATAGAATGGTATCCTCGTCGTAGCGAAATCTGGCGAGACATCG |
| | | (SEQ ID NO: 89) |
| | ft | CCATATTCAGCCTTTGAGAGTCTGGCTAAGTGTGGACAGAAATAGTTATATTCAAGC |
| | | ATATTTCTGTCCACACTTAGCCAGGCGAAATCTGGCGAGACATCG |
| | | (SEQ ID NO: 90) |
| | p38a | CCATATTCAGCCTTTGAGAGTAAGGATGTAAAGTGAACACAATAGTTATATTCAAGCA |
| | | TATTGTGTTCACTTTACATCCTTGCGAAATCTGGCGAGACATCG |
| | | (SEQ ID NO: 91) |
| | put | CCATATTCAGCCTTTGAGAGTCTCACCGAGACTTCAAGTCTATAGTTATATTCAAGC |
| | | ATATAGACTTGAAGTCTCGGTGAGGCGAAATCTGGCGAGACATCG |
| | | (SEQ ID NO: 92) |
| | shg | CCATATTCAGCCTTTGAGAGTAAGAGTGCAAATAGAATAGAATAGTTATATTCAAGC |
| | | ATATTCTATTCTATTTGCACTCTTGCGAAATCTGGCGAGACATCG |
| | | (SEQ ID NO: 93) |
| Cluster | p53 | CCATATTCAGCCTTTGAGAGTAGCGAGAATCCCAACAGTGTATAGTTATATTCAAGCA |
| 006.2 | | TATACACTGTTGGGATTCTCGCTGCGAAATCTGGCGAGACATCG |
| | | (SEQ ID NO: 94) |
| | Apc | CCATATTCAGCCTTTGAGAGTCTGGACGACCAGCTTCGATGATAGTTATATTCAAGCA |
| | | TATCATCGAAGCTGGTCGTCCAGGCGAAATCTGGCGAGACATCG |
| | | (SEQ ID NO: 95) |
| | ago | CCATATTCAGCCTTTGAGAGTAAGCCTTTGTATCTATGTCAATAGTTATATTCAAGC |
| | | ATATTGACATAGATACAAAGGCTTGCGAAATCTGGCGAGACATCG |
| | | (SEQ ID NO: 96) |
| | brm | CCATATTCAGCCTTTGAGAGTCTCGAAGCATCAGCACCTTAATAGTTATATTCAAGCA |
| | | TATTAAGGTGCTGATGCTTCGAGGCGAAATCTGGCGAGACATCG |
| | | (SEQ ID NO: 97) |
| | ft | CCATATTCAGCCTTTGAGAGTAGGTATGCGGGATCACTACGATAGTTATATTCAAGCA |
| | | TATCGTAGTGATCCCGCATACCTGCGAAATCTGGCGAGACATCG |
| | | (SEQ ID NO: 98) |
| | p38a | CCATATTCAGCCTTTGAGAGTATCGGTCTGCTGGACATATTCTAGTTATATTCAAGCA |
| | | TAGAATATGTCCAGCAGACCGATGCGAAATCTGGCGAGACATCG |
| | | (SEQ ID NO: 99) |
| | put | CCATATTCAGCCTTTGAGAGTCACGGACATGCACGAATGCAATAGTTATATTCAAGCA |
| | | TATTGCATTCGTGCATGTCCGTGGCGAAATCTGGCGAGACATCG |
| | | (SEQ ID NO: 100) |
| | shg | CCATATTCAGCCTTTGAGAGTTGTCCAGAAGCTACCACTGCATAGTTATATTCAAGCA |
| | | TATGCAGTGGTAGCTTCTGGACAGCGAAATCTGGCGAGACATCG |
| | | (SEQ ID NO: 101) |

TABLE 3B
Spacer sequences

| spacer | derived | |
|---|---|---|
| name | from | sequence |
| G-WAL F | Vector | actctgaatagggaattggg |
| | | aattgagatctgttctaga |
| | | (SEQ ID NO: 102) |
| G39.1 | miR-1 | agtagtgccaccaaaagtta |
| | | gccgcgttgtggaaaatcc |
| | | (SEQ ID NO: 103) |
| G39.2 | miR-279 | gagggaaatggagaacgcaa |
| | | aaatcccattataatggaa |
| | | (SEQ ID NO: 104) |
| G39.3 | miR-7 | atgtgcttgatcgtaactcc |
| | | atccaaactcgatattaac |
| | | (SEQ ID NO: 105) |
| G39.4 | miR-8 | acaaataatgttgcaataac |
| | | cagttgaaaccaatggaat |
| | | (SEQ ID NO: 106) |
| G39.5 | miR-278 | aactaacccgttcacctgcga |
| | | caatttttaatctatttt |
| | | (SEQ ID NO: 107) |
| G39.6 | Ban | agaccacgatcgaaagaggaa |
| | | aaacggaaaacgaacgaa |
| | | (SEQ ID NO: 108) |
| G39.7 | miR-14 | ggactagttttcattattta |
| | | tcagccagcaccaacaaca |
| G-WAL R | Vector | tagcggccgcaagaattcagg |
| | | cgaga (SEQ ID NO: 109) |

TABLE 3C
Fully assembled cluster sequences

| | GWAL-F | p53 | G39.1 | apc | G39.2 | ago | G39.3 |
|---|---|---|---|---|---|---|---|
| 006. | actc | CCAT | acta | CCAT | Gagg | CCAT | atgt |
| 1 | tgaa | ATTC | gtgc | ATTC | gaaa | ATTC | gctt |
| | tagg | AGCC | cacc | AGCC | tgga | AGCC | gatc |
| | gaat | TTTG | aaaa | TTTG | gaac | TTTG | gtaa |
| | tggg | AGAG | gtta | AGAG | gcaa | AGAG | ctcc |
| | aatt | TCAA | gccg | TCTC | aaat | TTCC | atcc |
| | gaga | CGTG | cgtt | AAAG | ccca | GATG | aaac |
| | tctg | GACG | gtgg | TTGT | ttat | ACAA | tcga |
| | ttct | TTCA | aaaa | GCAA | aatg | TACA | tatt |
| | aga | GTTC | tcc | CTCT | gaa | CTTA | aac |
| | (SEQ | AATA | (SEQ | TATA | (SEQ | AATA | (SEQ |
| | ID | GTTA | ID | GTTA | ID | GTTA | ID |
| | NO: | TATT | NO: | TATT | NO: | TATT | NO: |
| | 110) | CAAG | 112) | CAAG | 114) | CAAG | 116) |
| | | CATA | | CATA | | CATA | |
| | | TTGA | | TAAG | | TTTA | |
| | | ACTG | | AGTT | | AGTG | |
| | | AACG | | GCAC | | TATT | |
| | | TCCA | | AACT | | GTCA | |
| | | CGTT | | TTGA | | TCGG | |
| | | GGCG | | GGCG | | AGCG | |
| | | AAAT | | AAAT | | AAAT | |
| | | CTGG | | CTGG | | CTGG | |
| | | CGAG | | CGAG | | CGAG | |
| | | ACAT | | ACAT | | ACAT | |
| | | CG | | CG | | CG(S | |
| | | (SEQ | | (SEQ | | EQID | |
| | | ID | | ID | | NO: | |
| | | NO: | | NO: | | 115) | |
| | | 111) | | 113) | | | |
| 006.2 | actc | CCAT | agta | CCAT | gagg | CCAT | atgt |
| | tgaa | ATTC | gtgc | ATTC | gaaa | ATTC | gctt |
| | tagg | AGCC | cacc | AGCC | tgga | AGCC | gatc |
| | gaat | TTTG | aaaa | TTTG | gaac | TTTG | gtaa |
| | tggg | AGAG | gtta | AGAG | gcaa | AGAG | ctcc |
| | aatt | TAGC | gccg | TCTG | aaat | TAAG | atcc |
| | gaga | GAGA | cgtt | GACG | ccca | CCTT | aaac |
| | tctg | ATCC | gtgg | ACCA | ttat | TGTA | tcga |
| | ttct | CAAC | aaaa | GCTT | aatg | TCTA | tatt |
| | aga | AGTG | tcc | CGAT | gaa( | TGTC | aac |
| | (SEQ | TATA | (SEQ | GATA | SEQI | AATA | (SEQ |
| | ID | GTTA | ID | GTTA | DNO: | GTTA | ID |
| | NO: | TATT | NO: | TATT | 121) | TATT | NO: |
| | 117) | CAAG | 119) | CAAG | | CAAG | 123) |
| | | CATA | | CATA | | CATA | |
| | | TACA | | TCAT | | TTGA | |
| | | CTGT | | CGAA | | CATA | |
| | | TGGG | | GCTG | | GATA | |
| | | ATTC | | GTCG | | CAAA | |
| | | TCGC | | TCCA | | GGCT | |
| | | TGCG | | GGCG | | TGCG | |
| | | AAAT | | AAAT | | AAAT | |
| | | CTGG | | CTGG | | CTGG | |
| | | CGAG | | CGAG | | CGAG | |
| | | ACAT | | ACAT | | ACAT | |
| | | CG | | CG | | CG | |
| | | (SEQ | | (SEQ | | (SEQ | |
| | | ID | | ID | | ID | |
| | | NO: | | NO: | | NO: | |
| | | 118) | | 120) | | 122) | |
| | brm | G39.5 | Ft | G39.6 | p38a | G39.7 | Put |
| 006.1 | CCAT | aact | CCAT | agac | CCAT | ggac | CCAT |
| | ATTC | aacc | ATTC | cacg | ATTC | tagt | ATTC |
| | AGCC | cgtt | AGCC | atcg | AGCC | tttc | AGCC |
| | TTTG | cacc | TTTG | aaag | TTTG | atta | TTTG |
| | AGAG | tgcg | AGAG | agga | AGAG | ttta | AGAG |
| | TTAC | acaa | TCTG | aaaa | TAAG | tcag | TCTC |
| | GACG | tttt | GCTA | cgga | GATG | ccag | ACCG |
| | AGGA | taat | AGTG | aaac | TAAA | cacc | AGAC |
| | TACC | ctat | TGGA | gaac | GTGA | aaca | TTCA |
| | ATTC | ttt | CAGA | gaa | ACAC | aca | AGTC |
| | TATA | (SEQ | AATA | (SEQ | AATA | (SEQ | TATA |
| | GTTA | ID | GTTA | ID | GTTA | ID | GTTA |
| | TATT | NO: | TATT | NO: | TATT | NO: | TATT |
| | CAAG | 125) | CAAG | 127) | CAAG | 129) | CAAG |
| | CATA | | CATA | | CATA | | CATA |
| | TAGA | | TTTC | | TTGT | | TAGA |
| | ATGG | | TGTC | | GTTC | | CTTG |
| | TATC | | CACA | | ACTT | | AAGT |
| | CTCG | | CTTA | | TACA | | CTCG |
| | TCGT | | GCCA | | TCCT | | GTGA |
| | AGCG | | GGCG | | TGCG | | GGCG |
| | AAAT | | AAAT | | AWTC | | AAAT |
| | CTGG | | CTGG | | TGGC | | CTGG |
| | CGAG | | CGAG | | GAGA | | CGAG |
| | ACAT | | ACAT | | CATC | | ACAT |
| | CG | | CG | | G | | CG |
| | (SEQ | | (SEQ | | (SEQ | | (SEQ |
| | ID | | ID | | ID | | ID |
| | NO: | | NO: | | NO: | | NO: |
| | 124) | | 126) | | 128) | | 130) |
| 006.2 | CCAT | aact | CCAT | agac | CCAT | ggac | CCAT |
| | ATTC | aacc | ATTC | cacg | ATTC | tagt | ATTC |
| | AGCC | cgtt | AGCC | atcg | AGCC | tttc | AGCC |
| | TTTG | cacc | TTTG | aaag | TTTG | atta | TTTG |
| | AGAG | tgcg | AGAG | agga | AGAG | ttta | AGAG |
| | TCTC | acaa | TAGG | aaaa | TATC | tcag | TCAC |
| | GAAG | tttt | TATG | cgga | GGTC | ccag | GGAC |
| | CATC | taat | CGGG | aaac | TGCT | cacc | ATGC |
| | AGCA | ctat | ATCA | gaac | GGAC | aaca | ACGA |
| | CCTT | ttt | CTAC | gaa | ATAT | aca | ATGC |
| | AATA | (SEQ | GATA | (SEQ | TCTA | (SEQ | AATA |
| | GTTA | ID | GTTA | ID | GTTA | ID | GTTA |
| | TATT | NO: | TATT | NO: | TATT | NO: | TATT |
| | CAAG | 132) | CAAG | 134) | CAAG | 136) | CAAG |
| | CATA | | CATA | | CATA | | CATA |
| | TTAA | | TCGT | | GAAT | | TTGC |
| | GGTG | | AGTG | | ATGT | | ATTC |
| | CTGA | | ATCC | | CCAG | | GTGC |
| | TGCT | | CGCA | | CAGA | | ATGT |
| | TCGA | | TACC | | CCGA | | CCGT |
| | GGCG | | TGCG | | TGCG | | GGCG |
| | AAAT | | AAAT | | AAAT | | AAAT |
| | CTGG | | CTGG | | CTGG | | CTGG |
| | CGAG | | CGAG | | CGAG | | CGAG |
| | ACAT | | ACAT | | ACAT | | ACAT |
| | CG | | CG | | CG | | CG |
| | (SEQ | | (SEQ | | (SEQ | | (SEQ |
| | ID | | ID | | ID | | ID |
| | NO: | | NO: | | NO: | | NO: |
| | 131) | | 133) | | 135) | | 137) |

| | G39.2 | shg | G39.4 | GWALR-Not |
|---|---|---|---|---|
| 006.1 | gagg | CCAT | acaa | tagcggccgcaagaattcaggcg |
| | gaaa | ATTC | ataa | aga |
| | tgga | AGCC | tgtt | (SEQ ID NO: 141) |
| | gaac | TTTG | gcaa | |
| | gcaa | AGAG | taac | |
| | aaat | TAAG | cagt | |
| | ccca | AGTG | tgaa | |
| | ttat | CAAA | acca | |
| | aatg | TAGA | atgg | |
| | gaa | ATAG | aat | |
| | (SEQ | AATA | (SEQ | |
| | ID | GTTA | ID | |
| | NO: | TATT | NO: | |
| | 138) | CAAG | 140) | |
| | | CATA | | |
| | | TTCT | | |
| | | ATTC | | |
| | | TATT | | |
| | | TGCA | | |
| | | CTCT | | |
| | | TGCG | | |
| | | AAAT | | |
| | | CTGG | | |
| | | CGAG | | |
| | | ACAT | | |
| | | CG | | |
| | | (SEQ | | |
| | | ID | | |
| | | NO: | | |
| | | 139) | | |
| 006.2 | gagg | CCAT | acaa | tagcggccgcaagaattcaggcg |
| | gaaa | ATTC | ataa | aga |
| | tgga | AGCC | tgtt | (SEQ ID NO: 145) |
| | gaac | TTTG | gcaa | |
| | gcaa | AGAG | taac | |
| | aaat | TTGT | cagt | |
| | ccca | CCAG | tgaa | |
| | ttat | AAGC | acca | |
| | aatg | TACC | atgg | |
| | gaa | ACTG | aat | |
| | (SEQ | CATA | (SEQ | |
| | ID | GTTA | ID | |
| | NO: | TATT | NO: | |
| | 142) | CAAG | 144) | |
| | | CATA | | |
| | | TGCA | | |
| | | GTGG | | |
| | | TAGC | | |
| | | TTCT | | |
| | | GGAC | | |
| | | AGCG | | |
| | | AAAT | | |
| | | CTGG | | |
| | | CGAG | | |
| | | ACAT | | |
| | | CG | | |
| | | (SEQ | | |
| | | ID | | |
| | | NO: | | |
| | | 143) | | |

| | |
|---|---|
| 006.1 | actctgaatagggaattgggaattgagatctgttctagaCCATA |
| | TTCAGCCTTTGAGAGTCAACGTGGACGTTCAGTTCAATAGTTAT |
| | ATTCAAGCATATTGAACTGAACGTCCACGTTGGCGAAATCTGGC |
| | GAGACATCGagtagtgccaccaaaagttagccgcgttgtggaaa |
| | atccCCATATTCAGCCTTTGAGAGTCTCAAAGTTGTGCAACTCT |
| | TATAGTTATATTCAAGCATATAAGAGTTGCACAACTTTGAGGCG |
| | AAATCTGGCGAGACATCGgagggaaatggagaacgcaaaaatcc |
| | cattataatggaaCCATATTCAGCCTTTGAGAGTTCCGATGACA |
| | ATACACTTAAATAGTTATATTCAAGCATATTTAAGTGTATTGTC |
| | ATCGGAGCGAAATCTGGCGAGACATCGatgtgcttgatcgtaac |
| | tccatccaaactcgatattaacCCATATTCAGCCTTTGAGAGTT |
| | ACGACGAGGATACCATTCTATAGTTATATTCAAGCATATAGAAT |
| | GGTATCCTCGTCGTAGCGAAATCTGGCGAGACATCGaactaacc |
| | cgttcacctgcgacaatttttaatctattttCCATATTCAGCCT |
| | TTGAGAGTCTGGCTAAGTGTGGACAGAAATAGTTATATTCAAGC |
| | ATATTTCTGTCCACACTTAGCCAGGCGAAATCTGGCGAGACATC |
| | GagaccacgatcgaaagaggaaaaacggaaaacgaacgaaCCAT |
| | ATTCAGCCTTTGAGAGTAAGGATGTAAAGTGAACACAATAGTTA |
| | TATTCAAGCATATTGTGTTCACTTTACATCCTTGCGAAATCTGG |
| | CGAGACATCGggactagttttcattatttatcagccagcaccaa |
| | caacaCCATATTCAGCCTTTGAGAGTCTCACCGAGACTTCAAGT |
| | CTATAGTTATATTCAAGCATATAGACTTGAAGTCTCGGTGAGGC |
| | GAAATCTGGCGAGACATCGgagggaaatggagaacgcaaaaatc |
| | ccattataatggaaCCATATTCAGCCTTTGAGAGTAAGAGTGCA |
| | AATAGAATAGAATAGTTATATTCAAGCATATTCTATTCTATTTG |
| | CACTCTTGCGAAATCTGGCGAGACATCGacaaataatgttgcaa |
| | taaccagttgaaaccaatggaattagcggccgcaagaattcagg |
| | cgaga |
| | (SEQ ID NO: 146) |
| 006.2 | actctgaatagggaattgggaattgagatctgttctagaCCATA |
| | TTCAGCCTTTGAGAGTAGCGAGAATCCCAACAGTGTATAGTTAT |
| | ATTCAAGCATATACACTGTTGGGATTCTCGCTGCGAAATCTGGC |
| | GAGACATCGagtagtgccaccaaaagttagccgcgttgtggaaa |
| | atccCCATATTCAGCCTTTGAGAGTCTGGACGACCAGCTTCGAT |
| | GATAGTTATATTCAAGCATATCATCGAAGCTGGTCGTCCAGGCG |
| | AAATCTGGCGAGACATCGgagggaaatggagaacgcaaaaatcc |
| | cattataatggaaCCATATTCAGCCTTTGAGAGTAAGCCTTTGT |
| | ATCTATGTCAATAGTTATATTCAAGCATATTGACATAGATACAA |
| | AGGCTTGCGAAATCTGGCGAGACATCGatgtgcttgatcgtaac |
| | tccatccaaactcgatattaacCCATATTCAGCCTTTGAGAGTC |
| | TCGAAGCATCAGCACCTTAATAGTTATATTCAAGCATATTAAGG |
| | TGCTGATGCTTCGAGGCGAAATCTGGCGAGACATCGaactaacc |
| | cgttcacctgcgacaatttttaatctattttCCATATTCAGCCT |
| | TTGAGAGTAGGTATGCGGGATCACTACGATAGTTATATTCAAGC |
| | ATATCGTAGTGATCCCGCATACCTGCGAAATCTGGCGAGACATC |
| | GagaccacgatcgaaagaggaaaaacggaaaacgaacgaaCCAT |
| | ATTCAGCCTTTGAGAGTATCGGTCTGCTGGACATATTCTAGTTA |
| | 7ATTCAAGCATAGAATATGTCCAGCAGACCGATGCGAAATCTGG |
| | CGAGACATCGggactagtttteattatttatcagccagcaccaa |
| | caacaCCATATTCAGCCTTTGAGAGTCACGGACATGCACGAATG |
| | CAATAGTTATATTCAAGCATATTGCATTCGTGCATGTCCGTGGC |
| | GAAATCTGGCGAGACATCGgagggaaatggagaacgcaaaaatc |
| | ccattataatggaaCCATATTCAGCCTTTGAGAGTTGTCCAGAA |
| | GCTACCACTGCATASTTATATTCAAGCATATGCAGTGGTAGCTT |
| | CTGGACAGCGAAATCTGGCGAGACATCGacaaataatgttgcaa |
| | taaccagttgaaaccaatggaattagcggccgcaagaattcagg |
| | cgaga |
| | (SEQ ID NO: 147) |

TABLE 4A
Single agent drug screen data (EP: raw experimental pupae numbers)

| | replicate | replicate | replicate | replicate | replicate | replicate | replicate | replicate | | | |
|---|---|---|---|---|---|---|---|---|---|---|---|
| | 1 | 2 | 3 | 4 | 5 | 6 | 7 | 8 | | | |
| | EP | EP | EP | EP | EP | EP | EP | EP | mean | SEM | N |
| abiraterone | 3 | 0 | 1 | 2 | 1 | 2 | 1 | 0 | 1.25 | 0.365963 | 8 |
| acetate | | | | | | | | | | | |
| Afatinib | 2 | 0 | 2 | 1 | 0 | 1 | 1 | 2 | 1.125 | 0.295048 | 8 |
| anastrozole | 2 | 0 | 0 | 0 | 2 | 0 | 2 | 0 | 0.75 | 0.365963 | 8 |
| Axitinib | 3 | 0 | 1 | 1 | 1 | 0 | 2 | 0 | 1 | 0.377964 | 8 |
| bendamustine | 1 | 2 | 0 | 1 | 5 | 0 | 1 | 0 | 1.25 | 0.590097 | 8 |
| HCL | | | | | | | | | | | |
| bortezomib | 1 | 1 | 0 | 2 | 0 | 4 | 1 | 0 | 1.125 | 0.47949 | 8 |
| Bosutinib | 0 | 1 | 0 | 0 | 0 | 0 | 0 | 0 | 0.125 | 0.125 | 8 |
| busulflex | 0 | 0 | 0 | 1 | 1 | 1 | 0 | 0 | 0.375 | 0.182981 | 8 |
| cabazitaxel | 0 | 0 | 1 | 0 | 1 | 0 | 1 | 0 | 0.375 | 0.182981 | 8 |
| Cabozantinib | 0 | 1 | 0 | 0 | 1 | 0 | 1 | 0 | 0.375 | 0.182981 | 8 |
| Capecitabine | 2 | 1 | 0 | 0 | 5 | 1 | 1 | 1 | 1.375 | 0.564975 | 8 |
| (xeloda) | | | | | | | | | | | |
| Carfilzomib | 0 | 2 | 0 | 0 | 2 | 0 | 1 | 2 | 0.875 | 0.350382 | 8 |
| Cinacalcet | 2 | 2 | 0 | 2 | 1 | 1 | 0 | 0 | 1 | 0.327327 | 8 |
| clofarabine | 0 | 1 | 0 | 4 | 2 | 2 | 0 | 0 | 1.125 | 0.515388 | 8 |
| crizotinib | 0 | 0 | 2 | 0 | 6 | 0 | 2 | 0 | 1.25 | 0.75 | 8 |
| Dabrafenib | 2 | 2 | 1 | 1 | 2 | 1 | 1 | 1 | 1.375 | 0.182981 | 8 |
| dasatinib | 2 | 2 | 0 | 0 | 2 | 2 | 2 | 1 | 1.375 | 0.323899 | 8 |
| docetaxel | 0 | 0 | 0 | 0 | 0 | 1 | 0 | 0 | 0.125 | 0.125 | 8 |
| doxorubicin | 2 | 0 | 1 | 1 | 2 | 2 | 3 | 0 | 1.375 | 0.375 | 8 |
| ellence | 3 | 0 | 1 | 1 | 1 | 1 | 0 | 1 | 1 | 0.327327 | 8 |
| Enzalutamide | 0 | 4 | 5 | 2 | 5 | 0 | 2 | 1 | 2.375 | 0.730399 | 8 |
| erlotinib | 2 | 0 | 0 | 0 | 0 | 2 | 1 | 1 | 0.75 | 0.313392 | 8 |
| Everolimus | 0 | 1 | 1 | 0 | 0 | 2 | 0 | 0 | 0.5 | 0.267261 | 8 |
| exemestane | 3 | 0 | 0 | 2 | 0 | 0 | 2 | 0 | 0.875 | 0.440677 | 8 |
| (aromasin) | | | | | | | | | | | |
| flutamide | 0 | 1 | 0 | 0 | 1 | 0 | 0 | 0 | 0.25 | 0.163663 | 8 |
| fulvestrant | 3 | 2 | 1 | 1 | 2 | 3 | 0 | 0 | 1.5 | 0.422577 | 8 |
| gefitinib | 2 | 1 | 1 | 0 | 2 | 1 | 1 | 1 | 1.125 | 0.226582 | 8 |
| gemcitabine | 1 | 0 | 2 | 1 | 1 | 1 | 0 | 0 | 0.75 | 0.25 | 8 |
| imatinib | 0 | 2 | 0 | 0 | 1 | 1 | 1 | 2 | 0.875 | 0.295048 | 8 |
| Irinotecan | 0 | 4 | 0 | 0 | 3 | 0 | 0 | 0 | 0.875 | 0.580563 | 8 |
| (camptosar) | | | | | | | | | | | |
| lapatinib | 2 | 2 | 1 | 2 | 5 | 1 | 1 | 0 | 1.75 | 0.526104 | 8 |
| Lenalidomide | 1 | 1 | 3 | 2 | 1 | 1 | 1 | 0 | 1.25 | 0.313392 | 8 |
| Letrozole | 2 | 3 | 1 | 0 | 2 | 0 | 3 | 0 | 1.375 | 0.460493 | 8 |
| nelarabine | 1 | 1 | 3 | 0 | 0 | 0 | 0 | 0 | 0.625 | 0.375 | 8 |
| nilotinib | 2 | 1 | 1 | 0 | 0 | 0 | 0 | 0 | 0.5 | 0.267261 | 8 |
| pamidronate | 1 | 1 | 0 | 0 | 3 | 2 | 0 | 0 | 0.875 | 0.398098 | 8 |
| Pazopanib | 0 | 0 | 0 | 0 | 1 | 1 | 0 | 0 | 0.25 | 0.163663 | 8 |
| pemetrexed | 0 | 2 | 1 | 0 | 0 | 4 | 3 | 0 | 1.25 | 0.559017 | 8 |
| DiNA | | | | | | | | | | | |
| Pomalidonude | 0 | 0 | 2 | 3 | 0 | 0 | 0 | 0 | 0.625 | 0.419928 | 8 |
| Ponatinib | 0 | 1 | 3 | 1 | 1 | 1 | 0 | 0 | 0.875 | 0.350382 | 8 |
| Rapamycin | 0 | 1 | 1 | 0 | 0 | 2 | 1 | 1 | 0.75 | 0.25 | 8 |
| (sirolimus) | | | | | | | | | | | |
| Regorafenib | 0 | 0 | 0 | 1 | 0 | 0 | 0 | 1 | 0.25 | 0.163663 | 8 |
| sorafenib | 0 | 3 | 1 | 2 | 0 | 2 | 2 | 0 | 1.25 | 0.411877 | 8 |
| Sunitinib | 0 | 0 | 1 | 1 | 2 | 2 | 2 | 0 | 1 | 0.327327 | 8 |
| malate | | | | | | | | | | | |
| Tamoxifen | 0 | 1 | 0 | 1 | 1 | 1 | 2 | 0 | 0.75 | 0.25 | 8 |
| (nolvadex) | | | | | | | | | | | |
| temsirolimus | 1 | | 1 | 1 | 0 | 2 | 0 | 0 | 0.714862 | 0.285714 | 7 |
| topotecan | 0 | 0 | 0 | 0 | 0 | 0 | 0 | 0 | 0 | 0 | 8 |
| HCL | | | | | | | | | | | |
| Trametinib | 2 | 2 | 2 | 1 | 3 | 2 | 2 | 1 | 1.875 | 0.226582 | 8 |
| vandetanib | 1 | 0 | 1 | 2 | 0 | 1 | 3 | 0 | 1 | 0.377964 | 8 |
| vemurafenib | 1 | 1 | 1 | 2 | 1 | 1 | 2 | 1 | 1.25 | 0.163663 | 8 |
| Vismodegib | 2 | 3 | 0 | 0 | 0 | 1 | 3 | 0 | 1.125 | 0.47949 | 8 |
| vorinostat | 1 | 0 | 1 | 0 | 1 | 1 | 2 | 0 | 0.75 | 0.25 | 8 |
| zoledronic | 1 | 0 | 2 | 0 | 1 | 0 | 1 | 0 | 0.625 | 0.263052 | 8 |
| acid | | | | | | | | | | | |
| ibrutinib | 0 | 0 | 1 | 0 | 0 | 1 | 0 | 0 | 0.25 | 0.163663 | 8 |
| Idelalisib | 2 | 1 | 2 | 0 | 0 | 1 | 1 | 0 | 0.875 | 0.295048 | 8 |
| Belinostat | 2 | 0 | 1 | 2 | 0 | 0 | 1 | 0 | 0.75 | 0.313392 | 8 |
| Ceritinib | 1 | 3 | 4 | 2 | 0 | 0 | 2 | 1 | 1.625 | 0.497763 | 8 |
| Nintedanib | 1 | 3 | 1 | 2 | 2 | 0 | 1 | 0 | 1.25 | 0.365963 | 8 |
| Olaparib | 3 | 1 | 1 | 1 | 1 | 0 | 2 | 0 | 1.125 | 0.350382 | 8 |
| Lenvatinib | 2 | 1 | 0 | 0 | 0 | 0 | 1 | 0 | 0.5 | 0.267261 | 8 |
| Panobinostat | 1 | 2 | 0 | 2 | 0 | 1 | 2 | 0 | 1 | 0.327327 | 8 |
| Palbociclib | 0 | 2 | 0 | 2 | 1 | 1 | 0 | 0 | 0.75 | 0.313392 | 8 |
| Ruxolitinib | 0 | 0 | 0 | 0 | 0 | 1 | 0 | 0 | 0.125 | 0.125 | 8 |
| Alectinib | 1 | 0 | 0 | 0 | 1 | 0 | 0 | 0 | 0.25 | 0.163663 | 8 |
| Plain Food | 5 | 1 | 1 | 2 | 1 | 3 | 1 | 1 | 1.3125 | 0.338117 | 16 |
| Plain Food | 3 | 0 | 0 | 1 | 1 | 1 | 0 | 0 | | | |
| DMSO | 1 | 2 | 0 | 0 | 1 | 0 | 1 | 0 | 0.916667 | 0.154235 | 48 |
| DMSO | 3 | 1 | 1 | 0 | 1 | 1 | 0 | 0 | | | |
| DMSO | 2 | 2 | 1 | 3 | 0 | 3 | 1 | 1 | | | |
| DMSO | 1 | 0 | 2 | 0 | 2 | 1 | 0 | 0 | | | |
| DMSO | 0 | 0 | 0 | 1 | 0 | 1 | 1 | 0 | | | |
| DMSO | 5 | 1 | 2 | 1 | 0 | 0 | 0 | 1 | | | |

TABLE 4B
Trametinib combination drug screen data (EP: raw experimental pupae numbers)

| | replicate | replicate | replicate | replicate | replicate | replicate | replicate | replicate | | | |
|---|---|---|---|---|---|---|---|---|---|---|---|
| | 1 | 2 | 3 | 4 | 5 | 6 | 7 | 8 | | | |
| | EP | EP | EP | EP | EP | EP | EP | EP | mean | SEM | N |
| amlodipine besylate | 0 | 0 | 0 | 1 | 1 | 0 | 0 | 0 | 0.25 | 0.163663 | 8 |
| apremilast | 0 | 0 | 2 | 0 | 3 | 0 | 0 | 0 | 0.625 | 0.419928 | 8 |
| aripiprazole | 0 | 0 | 1 | 0 | 0 | 2 | 1 | 2 | 0.75 | 0.313392 | 8 |
| brexpiprazole | 0 | 0 | 1 | 1 | 2 | 0 | 3 | 1 | 1 | 0.377964 | 8 |
| brivaracetam | 0 | 0 | 2 | 0 | 2 | 1 | 1 | 1 | 0.875 | 0.295048 | 8 |
| cariprazine | 2 | 0 | 0 | 2 | 2 | 0 | 0 | 3 | 1.125 | 0.440677 | 8 |
| hydrochloride | | | | | | | | | | | |
| cholic acid | 1 | 0 | 2 | 1 | 1 | 1 | 3 | 2 | 1.375 | 0.323899 | 8 |
| clozapine | 1 | 0 | 0 | 0 | 3 | 2 | 1 | 2 | 1.125 | 0.398098 | 8 |
| cobimetinib | 1 | 1 | 2 | 0 | 1 | 0 | 0 | 0 | 0.625 | 0.263052 | 8 |
| cyproheptadine HCl | 0 | 0 | 0 | 0 | 3 | 0 | 1 | 3 | 0.875 | 0.47949 | 8 |
| dapagliflozin | 0 | 1 | 1 | 1 | 3 | 5 | 2 | 0 | 1.625 | 0.595744 | 8 |
| empagliflozin | 4 | 0 | 0 | 2 | 5 | 2 | 1 | 2 | 2 | 0.626783 | 8 |
| Entresto (LCZ696) | 0 | 0 | 2 | 2 | 2 | 1 | 2 | 6 | 1.875 | 0.666481 | 8 |
| flibanserin | 0 | 0 | 3 | 0 | 0 | 0 | 1 | 0 | 0.5 | 0.377964 | 8 |
| fluoxetine | 2 | 2 | 0 | 0 | 7 | 1 | 0 | 1 | 1.625 | 0.822398 | 8 |
| Fluticasone | 0 | 0 | 1 | 0 | 2 | 0 | 0 | 1 | 0.5 | 0.267261 | 8 |
| propionate | | | | | | | | | | | |
| haloperidol | 0 | 0 | 2 | 2 | 2 | 1 | 1 | 1 | 1.125 | 0.295048 | 8 |
| indomethacin | 0 | 0 | 0 | 0 | 1 | 0 | 0 | 0 | 0.125 | 0.125 | 8 |
| ivabradine HCl | 0 | 0 | 0 | 0 | 2 | 1 | 0 | 0 | 0.375 | 0.263052 | 8 |
| ivacaftor | 0 | 0 | 7 | 0 | 3 | 3 | 1 | 2 | 2 | 0.845154 | 8 |
| ixazomib | 1 | 1 | 1 | 0 | 1 | 2 | 0 | 0 | 0.75 | 0.25 | 8 |
| lesinurad sodium | 0 | 0 | 1 | 0 | 1 | 2 | 1 | 0 | 0.625 | 0.263052 | 8 |
| lumacaftor | 0 | 4 | 4 | 1 | 2 | 1 | 1 | 4 | 2.125 | 0.580563 | 8 |
| meloxicam | 0 | 0 | 0 | 3 | 1 | 1 | 0 | 1 | 0.75 | 0.365963 | 8 |
| metformin HCl | 0 | 0 | 0 | 1 | 1 | 0 | 2 | 2 | 0.75 | 0.313392 | 8 |
| nelfinavir | 0 | 0 | 0 | 1 | 4 | 0 | 0 | 0 | 0.625 | 0.497763 | 8 |
| Methanesulfonate | | | | | | | | | | | |
| Salt | | | | | | | | | | | |
| osimertinib | 0 | 0 | 0 | 0 | 0 | 0 | 3 | 1 | 0.5 | 0.377964 | 8 |
| paroxetine HCl | 0 | 0 | 0 | 0 | 1 | 2 | 0 | 1 | 0.5 | 0.267261 | 8 |
| perindropil erbumine | 0 | 0 | 3 | 3 | 5 | 1 | 2 | 2 | 2 | 0.597614 | 8 |
| pyrvinium | 4 | 1 | 6 | 4 | 2 | 1 | 3 | 0 | 2.625 | 0.705527 | 8 |
| selexipag | 3 | 0 | 1 | 0 | 2 | 4 | 4 | 2 | 2 | 0.566947 | 8 |
| sonidegib | 0 | 1 | 5 | 2 | 3 | 1 | 3 | 1 | 2 | 0.566947 | 8 |
| diphosphate salt | | | | | | | | | | | |
| sumatriptan | 0 | 0 | 1 | 1 | 1 | 1 | 1 | 1 | 0.75 | 0.163663 | 8 |
| succinate | | | | | | | | | | | |
| suvorexant | 0 | 0 | 3 | 1 | 5 | 1 | 1 | 3 | 1.75 | 0.61962 | 8 |
| Tacrolimus | 0 | 0 | 0 | 3 | 0 | 1 | 2 | 0 | 0.75 | 0.411877 | 8 |
| tofacitinib | 1 | 0 | 1 | 0 | 2 | 1 | 0 | 0 | 0.625 | 0.263052 | 8 |
| topiramate | 0 | 0 | 0 | 0 | 3 | 3 | 1 | 0 | 0.875 | 0.47949 | 8 |
| vorapaxar | 0 | 0 | 1 | 0 | 3 | 0 | 0 | 0 | 0.5 | 0.377964 | 8 |
| Voriconazole | 0 | 0 | 0 | 1 | 2 | 1 | 0 | 1 | 0.625 | 0.263052 | 8 |
| acipimox | 0 | 1 | 0 | 1 | 1 | 1 | 0 | 4 | 1 | 0.46291 | 8 |
| amifostine | 1 | 1 | 1 | 0 | 1 | 6 | 0 | 0 | 1.25 | 0.700765 | 8 |
| benztropine | 0 | 0 | 4 | 2 | 3 | 1 | 0 | 3 | 1.652 | 0.564975 | 8 |
| chloipromazine HCl | 0 | 1 | 1 | 1 | 1 | 2 | 0 | 3 | 1.125 | 0.350382 | 8 |
| famotidine | 0 | 0 | 0 | 0 | 1 | 2 | 0 | 2 | 0.625 | 0.323899 | 8 |
| fluvastatin | 0 | 0 | 0 | 0 | 0 | 0 | 0 | 2 | 0.25 | 0.25 | 8 |
| gemfibrozil | 0 | 1 | 0 | 0 | 3 | 0 | 0 | 0 | 0.5 | 0.377964 | 8 |
| ibandronate | 2 | 0 | 1 | 6 | 3 | 1 | 0 | 4 | 2.125 | 0.742522 | 8 |
| indapamide | 0 | 0 | 1 | 0 | 2 | 2 | 0 | 4 | 1.125 | 0.515388 | 8 |
| megestrol acetate | 0 | 1 | 1 | 0 | 1 | 2 | 1 | 2 | 1 | 0.267261 | 8 |
| nomifensine maleate | 0 | 0 | 0 | 0 | 1 | 4 | 0 | 1 | 0.75 | 0.40999 | 8 |
| pranaprofen | 0 | 1 | 1 | 1 | 1 | 3 | 1 | 3 | 1.375 | 0.375 | 8 |
| sisomicin | 1 | 1 | 1 | 1 | 2 | 1 | 0 | 0 | 0.875 | 0.226582 | 8 |
| sulindac | 0 | 0 | 1 | 2 | 1 | 1 | 0 | 2 | 0.875 | 0.295048 | 8 |
| thalidomide | 1 | 1 | 0 | 1 | 0 | 0 | 0 | 0 | 0.375 | 0.182981 | 8 |
| zonisamide | 0 | 2 | 0 | 1 | 3 | 1 | 1 | 0 | 1 | 0.377964 | 8 |
| camylofine | 0 | 0 | 1 | 0 | 6 | 1 | 0 | 0 | 1 | 0.731925 | 8 |
| dihydrochloride | | | | | | | | | | | |
| thonzonium bromide | 0 | 0 | 1 | 0 | 2 | 1 | 1 | 3 | 1 | 0.377964 | 8 |
| Plain Food | 0 | 0 | 0 | 1 | 0 | 0 | 0 | 0 | 0.125 | 0.085391 | 16 |
| Plain Food | 0 | 0 | 0 | 0 | 0 | 0 | 0 | 1 | | | |
| Trametinib alone | 0 | 0 | 0 | 0 | 1 | 1 | 0 | 0 | 0.291667 | 0.094776 | 24 |
| Trametinib alone | 1 | 0 | 0 | 0 | 1 | 1 | 0 | 0 | | | |
| Trametinib alone | 0 | 1 | 0 | 1 | 0 | 0 | 0 | 0 | | | |
| DMSO | 0 | 0 | 0 | 0 | 0 | 0 | 0 | 0 | 0 | 0 | 32 |
| DMSO | 0 | 0 | 0 | 0 | 0 | 0 | 0 | 0 | | | |
| DMSO | 0 | 0 | 0 | 0 | 0 | 0 | 0 | 0 | | | |
| DMSO | 0 | 0 | 0 | 0 | 0 | 0 | 0 | 0 | | | |

TABLE 4C
Number of experimental (EP) and control (CP) for graphs presented in FIG. 2C and 2D

| | replicate | | replicate | | replicate | | replicate | | replicate | | replicate | | replicate | | replicate | |
|---|---|---|---|---|---|---|---|---|---|---|---|---|---|---|---|---|
| | 1 | | 2 | | 3 | | 4 | | 5 | | 6 | | 7 | | 8 | |
| | CP | EP | CP | EP | CP | EP | CP | EP | CP | EP | CP | EP | CP | EP | CP | EP |
| 1 μM | 20 | 6 | 31 | 7 | 22 | 2 | 21 | 3 | 30 | 8 | 12 | 10 | 23 | 8 | 17 | 6 |
| Trametinib + | | | | | | | | | | | | | | | | |
| 1 μM | | | | | | | | | | | | | | | | |
| Ibandronate | | | | | | | | | | | | | | | | |
| 1 μM | 21 | 5 | 28 | 5 | 20 | 10 | 24 | 8 | 26 | 7 | 20 | 5 | 19 | 7 | 29 | 5 |
| Trametinib + | | | | | | | | | | | | | | | | |
| 0.1 μM | | | | | | | | | | | | | | | | |
| Ibandronate | | | | | | | | | | | | | | | | |
| 1 μM | 14 | 5 | 18 | 4 | 19 | 6 | 24 | 4 | 21 | 5 | 26 | 10 | 40 | 5 | 29 | 5 |
| Trametinib + | | | | | | | | | | | | | | | | |
| 0.01 μM | | | | | | | | | | | | | | | | |
| Ibandronate | | | | | | | | | | | | | | | | |
| 1 μM | 30 | 3 | 33 | 2 | 23 | 5 | 26 | 3 | 29 | 4 | 13 | 2 | 20 | 3 | 28 | 3 |
| Trametinib | | | | | | | | | | | | | | | | |
| 1 μM | 22 | 8 | 17 | 5 | 14 | 2 | 39 | 6 | 23 | 2 | 16 | 2 | 18 | 0 | 24 | 4 |
| Trametinib | | | | | | | | | | | | | | | | |
| 1 μM | 16 | 2 | 27 | 4 | 22 | 10 | 21 | 7 | 29 | 3 | 23 | 1 | 23 | 2 | 24 | 0 |
| Trametinib | | | | | | | | | | | | | | | | |
| 1 μM | 20 | 3 | 33 | 2 | 10 | 1 | 31 | 4 | 30 | 3 | 31 | 1 | 25 | 1 | 33 | 4 |
| Trametinib | | | | | | | | | | | | | | | | |
| 1 μM | 16 | 1 | 31 | 4 | 24 | 0 | 36 | 1 | 24 | 5 | 25 | 3 | 13 | 4 | 37 | 9 |
| Trametinib | | | | | | | | | | | | | | | | |
| 0.1% DMSO | 18 | 1 | 35 | 0 | 21 | 1 | 39 | 0 | 9 | 3 | 15 | 2 | 25 | 4 | 30 | 4 |
| 0.1% DMSO | 24 | 0 | 15 | 0 | 12 | 0 | 32 | 0 | 20 | 1 | 14 | 5 | 26 | 2 | 30 | 0 |
| 0.1% DMSO | 29 | 0 | 28 | 1 | 32 | 0 | 30 | 0 | 27 | 1 | 36 | 0 | 17 | 2 | 29 | 3 |
| 0.1% DMSO | 31 | 0 | 18 | 2 | 19 | 1 | 14 | 0 | 34 | 1 | 17 | 2 | 18 | 2 | 16 | 2 |
| 0.1% DMSO | 49 | 1 | 30 | 1 | 25 | 0 | 31 | 0 | 18 | 8 | 20 | 2 | 21 | 2 | 29 | 3 |
| 0.1% DMSO | 23 | 0 | 20 | 0 | 28 | 0 | 29 | 0 | 31 | 0 | 30 | 0 | 30 | 0 | 41 | 3 |
| 0.1% DMSO | 22 | 2 | 38 | 3 | 16 | 5 | 11 | 0 | 27 | 0 | 25 | 0 | 38 | 1 | 25 | 0 |
| 0.1% DMSO | 30 | 1 | 24 | 3 | 34 | 2 | 29 | 1 | 16 | 0 | | | | | | |
| 1 μM | 12 | 3 | 8 | 1 | 14 | 1 | 16 | 2 | 9 | 6 | 15 | 2 | 9 | 1 | 21 | 3 |
| Trametinib + | | | | | | | | | | | | | | | | |
| 7 μM | | | | | | | | | | | | | | | | |
| zoledronate | | | | | | | | | | | | | | | | |
| 1 μM | 12 | 8 | 7 | 4 | 11 | 4 | 8 | 7 | 16 | 1 | 7 | 0 | 6 | 1 | 12 | 3 |
| Trametinib + | | | | | | | | | | | | | | | | |
| 0.7 μM | | | | | | | | | | | | | | | | |
| zoledronate | | | | | | | | | | | | | | | | |
| 1 μM | 11 | 2 | 10 | 2 | 13 | 1 | 18 | 6 | 12 | 3 | 4 | 1 | 9 | 4 | 12 | 0 |
| Trametinib + | | | | | | | | | | | | | | | | |
| 0.07 μM | | | | | | | | | | | | | | | | |
| zoledronate | | | | | | | | | | | | | | | | |
| 1 μM | 16 | 1 | 14 | 1 | 15 | 2 | 8 | 2 | 11 | 2 | 16 | 1 | 15 | 3 | 10 | 1 |
| Trametinib | | | | | | | | | | | | | | | | |
| 0.1% DMSO | 12 | 0 | 17 | 0 | 7 | 2 | 18 | 1 | 10 | 0 | 14 | 1 | 5 | 1 | 13 | 3 |

TABLE 5
Tumor measurements

| | | | Time | Time | Time | Time | Time | Time |
|---|---|---|---|---|---|---|---|---|
| Lesion | Lesion type/location | Baseline | point 1 | point 2 | point 3 | point 4 | point 5 | point 6 |
| TARGET LESIONS | | | | | | | | |
| 1 | Left supraclavicular | 28 × 30 | 15 × 16 | 12 × 16 | 10 × 16 | 9 × 16 | 10 × 16 | 12 × 22 |
| | nodal mass | mm | mm | mm | mm | mm | mm | mm |
| 2 | RLL lung nodule | 13 × 9 | 13 × 10 | 14 × 12 | 16 × 13 | 16 × 15 | 26 × 18 | 26 × 17 |
| | | mm | mm | mm | mm | mm | mm | nm |
| 3 | RLL lung nodule | 15 × 11 | 5 × 4 | 10 × 8 | 14 × 11 | 16 × 13 | 21 × 18 | 21 × 20 |
| | | mm | mm | mm | mm | mm | mm | |
| 4 | Right retroperitoneal | 18 × 24 | 8 × 13 | 5 × 14 | 5 × 10 | 5 × 11 | 5 × 7 | 5 × 8 |
| | LN | mm | mm | mm | mm | mm | mm | nm |
| 5 | | | | | | | | |
| Sum Largest Diameter | | 74 mm | 41 mm | 41 mm | 45 mm | 46 mm | 62 mm | 64 mm |
| (SLD)* | | | | | | | | |
| Nadir** | | | | 41 mm | 41 mm | 41 mm | 41 mm | 41 mm |
| Percentage Change from | | NA | −44.6% | −44.6% | −39.1% | −37.8% | −16.2% | −13.5% |
| Baseline | | | | | | | | |
| Percentage Change from | | NA | | 0.0% | 9.7% | 12.1% | 51.2% | 56.1% |
| Nadir | | | | | | | | |
| NON TARGET LESIONS | | | | | | | | |
| 1 | Left axillary LN | 16 × 17 | 7 × 7 | 7 × 9 | 7 × 7 | 5 × 9 | 10 × 21 | 21 × 22 |
| | | mm | mm | mm | mm | mm | mm | |
| 2 | LUL lung nodule | 10 × 8 | 5 × 3 | 9 × 7 | 10 × 9 | 13 × 12 | 17 × 15 | 16 × 15 |
| | | mm | mm | mm | mm | mm | mm | |
| 3 | Aortocaval LN | 19 × 19 | 15 × 15 | 15 × 17 | 13 × 14 | 12 × 22 | 21 × 24 | 25 × 25 |
| | | mm | mm | mm | mm | mm | mm | |
| 4 | Left paraaortic LN | 17 × 21 | 9 × 9 | 5 × 9 | 5 × 7 | 6 × 9 | 8 × 11 | 7 × 8 |
| | | mm | mm | mm | mm | mm | mm | |
| 5 | Right external iliac LN | 16 × 24 | 7 × 9 | 7 × 9 | 8 × 10 | 9 × 17 | 18 × 23 | 20 × 24 |
| | | mm | mm | mm | mm | mm | mm | |
| NEW LESIONS | | | | | | | | |
| 1 | Left perianatomotic soft | | | 16 × 15 | 19 × 16 | 22 × 18 | 28 × 26 | 34 × 26 |
| | tissue nodule | | | mm | mm | mm | mm | |
| 2 | Portocaval nodal mass | | | 15 × 53 | 21 × 65 | 27 × 85 | 24 × 85 | 34 × 74 |
| | | | | mm | mm | min | mm | |
| 3 | Left perirenal soft tissue | | | | | | 32 × 23 | 40 × 36 |
| | nodules | | | | | | mm | |
| 4 | Aortopulmonary | | | | | | 15 × 18 | 15 × 18 |
| | window LN | | | | | | mm | |
| 5 | Pleural effusion and | | | | | | | Present |
| | ascites | | | | | | | |

*longest diameter for non-nodal lesions and short axis for nodes is used
**the smallest SLD during treatment)

The foregoing is not to be limited in scope by the specific embodiments described herein. Indeed, various modifications and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. A method for treating colorectal cancer, the method comprising administering to a human subject diagnosed with colorectal cancer a first composition comprising a mitogen-activated protein kinase/extracellular signal-regulated kinase (MAPK/ERK) kinase (MEK) inhibitor and a second composition comprising a bisphosphonate.

2. The method of claim 1, wherein the colorectal cancer is KRAS-mutant colorectal cancer.

3. The method of claim 1, wherein the colorectal cancer is KRAS-mutant colorectal adenocarcinoma cancer.

4. The method of claim 1, wherein the colorectal cancer is NRAS-mutant or HRAS mutant colorectal cancer.

5. The method of claim 1, wherein the colorectal cancer contains a gene isoform previously demonstrated to activate KRAS, HRAS, or NRAS.

6. The method of claim 1, wherein the MEK inhibitor is trametinib.

7. The method of claim 1, wherein the MEK inhibitor is trametinib dimethyl sulfoxide.

8. The method of claim 1, wherein the first composition is a tablet.

9. The method of claim 7, wherein the first composition is MEKINIST®.

10. The method of claim 1, wherein the MEK inhibitor is cobimetinib.

11. The method of claim 1, wherein the MEK inhibitor is cobimetinib fumarate.

12. (canceled)

13. The method of claim 11, wherein the first composition is COTELLIC®.

14. The method of claim 1, wherein the MEK inhibitor is binimetinib.

15. (canceled)

16. The method of claim 14, wherein the first composition is MEKTOVI®.

17. The method of claim 1, wherein the MEK inhibitor is CI-1040 (PD184352), PD0325901, Selumetinib (AZD6244), MEK162, AZD8330, TAK-733, GDC-0623, Refametinib (RDEAl 19; BAY 869766), Pimasertib (AS703026), R04987655 (CH4987655), R05126766, WX-554, HL-085, E6201, GDC-0623, or PD098059.

18. (canceled)

19. The method of claim 1, wherein the first composition is orally administered to the subject.

20. The method of claim 1, wherein the bisphosphanonate is etidronate, alendronate, risedronate, ibandronate, zoledronic acid, alendronate sodium, clodronate, tiludronate, pamidronate, neridronate, or olpadronate.

21. The method of claim 20, wherein the bisphosphonate is zoledronic acid.

22. The method of claim 21, wherein the second composition is Zometa®.

23. The method of claim 20, wherein the bisphosphonate is ibandronate.

24. The method of claim 23, wherein the second composition is BONIVA®.

25. The method of claim 1, wherein the second composition is administered to the subject intravenously or orally.

26. The method of claim 1, wherein the subject is unresponsive to other therapies approved for colorectal cancer.

27. The method of claim 1, wherein the dosage of the MEK inhibitor and the dosage of the bisphosphonate are the dosages approved by the U.S. Food and Drug Administration for any use.


SEQUENCE LISTING (147 sequences)

SEQ ID NO: 1 - DNA, 21 bp, Artificial Sequence
  hairpin
ccatattcagcctttgagagt

SEQ ID NO: 2 - DNA, 21 bp, Artificial Sequence
  hairpin
  misc_feature (1)..(21): n is a, c, g, or t
nnnnnnnnnnnnnnnnnnnnn

SEQ ID NO: 3 - DNA, 18 bp, Artificial Sequence
  hairpin
tagttatattcaagcata

SEQ ID NO: 4 - DNA, 21 bp, Artificial Sequence
  hairpin
  misc_feature (1)..(21): n is a, c, g, or t
nnnnnnnnnnnnnnnnnnnnn

SEQ ID NO: 5 - DNA, 21 bp, Artificial Sequence
  hairpin
gcgaaatctggcgagacatcg

SEQ ID NO: 6 - DNA, 21 bp, Artificial Sequence
  hairpin
ccatattcagcctttgagagt

SEQ ID NO: 7 - DNA, 21 bp, Artificial Sequence
  hairpin
caacgtggacgttcagttcaa

SEQ ID NO: 8 - DNA, 18 bp, Artificial Sequence
  hairpin
tagttatattcaagcata

SEQ ID NO: 9 - DNA, 21 bp, Artificial Sequence
  hairpin
ttgaactgaacgtccacgttg

SEQ ID NO: 10 - DNA, 21 bp, Artificial Sequence
  hairpin
gcgaaatctggcgagacatcg

SEQ ID NO: 11 - DNA, 21 bp, Artificial Sequence
  hairpin
ccatattcagcctttgagagt

SEQ ID NO: 12 - DNA, 21 bp, Artificial Sequence
  hairpin
ctcaaagttgtgcaactctta

SEQ ID NO: 13 - DNA, 18 bp, Artificial Sequence
  hairpin
tagttatattcaagcata

SEQ ID NO: 14 - DNA, 21 bp, Artificial Sequence
  hairpin
taagagttgcacaactttgag

SEQ ID NO: 15 - DNA, 21 bp, Artificial Sequence
  hairpin
gcgaaatctggcgagacatcg

SEQ ID NO: 16 - DNA, 21 bp, Artificial Sequence
  hairpin
ccatattcagcctttgagagt

SEQ ID NO: 17 - DNA, 21 bp, Artificial Sequence
  hairpin
tccgatgacaatacacttaaa

SEQ ID NO: 18 - DNA, 18 bp, Artificial Sequence
  hairpin
tagttatattcaagcata

SEQ ID NO: 19 - DNA, 21 bp, Artificial Sequence
  hairpin
tttaagtgtattgtcatcgga

SEQ ID NO: 20 - DNA, 21 bp, Artificial Sequence
  hairpin
gcgaaatctggcgagacatcg

SEQ ID NO: 21 - DNA, 21 bp, Artificial Sequence
  hairpin
ccatattcagcctttgagagt

SEQ ID NO: 22 - DNA, 21 bp, Artificial Sequence
  hairpin
tacgacgaggataccattcta

SEQ ID NO: 23 - DNA, 18 bp, Artificial Sequence
  hairpin
tagttatattcaagcata

SEQ ID NO: 24 - DNA, 21 bp, Artificial Sequence
  hairpin
tagaatggtatcctcgtcgta

SEQ ID NO: 25 - DNA, 21 bp, Artificial Sequence
  hairpin
gcgaaatctggcgagacatcg

SEQ ID NO: 26 - DNA, 21 bp, Artificial Sequence
  hairpin
ccatattcagcctttgagagt

SEQ ID NO: 27 - DNA, 21 bp, Artificial Sequence
  hairpin
ctggctaagtgtggacagaaa

SEQ ID NO: 28 - DNA, 18 bp, Artificial Sequence
  hairpin
tagttatattcaagcata

SEQ ID NO: 29 - DNA, 21 bp, Artificial Sequence
  hairpin
tttctgtccacacttagccag

SEQ ID NO: 30 - DNA, 21 bp, Artificial Sequence
  hairpin
gcgaaatctggcgagacatcg

SEQ ID NO: 31 - DNA, 21 bp, Artificial Sequence
  hairpin
ccatattcagcctttgagagt

SEQ ID NO: 32 - DNA, 21 bp, Artificial Sequence
  hairpin
aaggatgtaaagtgaacacaa

SEQ ID NO: 33 - DNA, 18 bp, Artificial Sequence
  hairpin
tagttatattcaagcata

SEQ ID NO: 34 - DNA, 21 bp, Artificial Sequence
  hairpin
ttgtgttcactttacatcctt

SEQ ID NO: 35 - DNA, 21 bp, Artificial Sequence
  hairpin
gcgaaatctggcgagacatcg

SEQ ID NO: 36 - DNA, 21 bp, Artificial Sequence
  hairpin
ccatattcagcctttgagagt

SEQ ID NO: 37 - DNA, 21 bp, Artificial Sequence
  hairpin
ctcaccgagacttcaagtcta

SEQ ID NO: 38 - DNA, 18 bp, Artificial Sequence
  hairpin
tagttatattcaagcata

SEQ ID NO: 39 - DNA, 21 bp, Artificial Sequence
  hairpin
tagacttgaagtctcggtgag

SEQ ID NO: 40 - DNA, 21 bp, Artificial Sequence
  hairpin
gcgaaatctggcgagacatcg

SEQ ID NO: 41 - DNA, 21 bp, Artificial Sequence
  hairpin
ccatattcagcctttgagagt

SEQ ID NO: 42 - DNA, 21 bp, Artificial Sequence
  hairpin
aagagtgcaaatagaatagaa

SEQ ID NO: 43 - DNA, 18 bp, Artificial Sequence
  hairpin
tagttatattcaagcata

SEQ ID NO: 44 - DNA, 21 bp, Artificial Sequence
  hairpin
ttctattctatttgcactctt

SEQ ID NO: 45 - DNA, 21 bp, Artificial Sequence
  hairpin
gcgaaatctggcgagacatcg

SEQ ID NO: 46 - DNA, 21 bp, Artificial Sequence
  hairpin
ccatattcagcctttgagagt

SEQ ID NO: 47 - DNA, 21 bp, Artificial Sequence
  hairpin
agcgagaatcccaacagtgta

SEQ ID NO: 48 - DNA, 18 bp, Artificial Sequence
  hairpin
tagttatattcaagcata

SEQ ID NO: 49 - DNA, 21 bp, Artificial Sequence
  hairpin
tacactgttgggattctcgct

SEQ ID NO: 50 - DNA, 21 bp, Artificial Sequence
  hairpin
gcgaaatctggcgagacatcg

SEQ ID NO: 51 - DNA, 21 bp, Artificial Sequence
  hairpin
ccatattcagcctttgagagt

SEQ ID NO: 52 - DNA, 21 bp, Artificial Sequence
  hairpin
ctggacgaccagcttcgatga

SEQ ID NO: 53 - DNA, 18 bp, Artificial Sequence
  hairpin
tagttatattcaagcata

SEQ ID NO: 54 - DNA, 21 bp, Artificial Sequence
  hairpin
tcatcgaagctggtcgtccag

SEQ ID NO: 55 - DNA, 21 bp, Artificial Sequence
  hairpin
gcgaaatctggcgagacatcg

SEQ ID NO: 56 - DNA, 21 bp, Artificial Sequence
  hairpin
ccatattcagcctttgagagt

SEQ ID NO: 57 - DNA, 21 bp, Artificial Sequence
  hairpin
aagcctttgtatctatgtcaa

SEQ ID NO: 58 - DNA, 18 bp, Artificial Sequence
  hairpin
tagttatattcaagcata

SEQ ID NO: 59 - DNA, 21 bp, Artificial Sequence
  hairpin
ttgacatagatacaaaggctt

SEQ ID NO: 60 - DNA, 21 bp, Artificial Sequence
  hairpin
gcgaaatctggcgagacatcg

SEQ ID NO: 61 - DNA, 21 bp, Artificial Sequence
  hairpin
ccatattcagcctttgagagt

SEQ ID NO: 62 - DNA, 21 bp, Artificial Sequence
  hairpin
ctcgaagcatcagcaccttaa

SEQ ID NO: 63 - DNA, 18 bp, Artificial Sequence
  hairpin
tagttatattcaagcata

SEQ ID NO: 64 - DNA, 21 bp, Artificial Sequence
  hairpin
ttaaggtgctgatgcttcgag

SEQ ID NO: 65 - DNA, 21 bp, Artificial Sequence
  hairpin
gcgaaatctggcgagacatcg

SEQ ID NO: 66 - DNA, 21 bp, Artificial Sequence
  hairpin
ccatattcagcctttgagagt

SEQ ID NO: 67 - DNA, 21 bp, Artificial Sequence
  hairpin
aggtatgcgggatcactacga

SEQ ID NO: 68 - DNA, 18 bp, Artificial Sequence
  hairpin
tagttatattcaagcata

SEQ ID NO: 69 - DNA, 21 bp, Artificial Sequence
  hairpin
tcgtagtgatcccgcatacct

SEQ ID NO: 70 - DNA, 21 bp, Artificial Sequence
  hairpin
gcgaaatctggcgagacatcg

SEQ ID NO: 71 - DNA, 21 bp, Artificial Sequence
  hairpin
ccatattcagcctttgagagt

SEQ ID NO: 72 - DNA, 21 bp, Artificial Sequence
  hairpin
atcggtctgctggacatattc

SEQ ID NO: 73 - DNA, 18 bp, Artificial Sequence
  hairpin
tagttatattcaagcata

SEQ ID NO: 74 - DNA, 21 bp, Artificial Sequence
  hairpin
gaatatgtccagcagaccgat

SEQ ID NO: 75 - DNA, 21 bp, Artificial Sequence
  hairpin
gcgaaatctggcgagacatcg

SEQ ID NO: 76 - DNA, 21 bp, Artificial Sequence
  hairpin
ccatattcagcctttgagagt

SEQ ID NO: 77 - DNA, 21 bp, Artificial Sequence
  hairpin
cacggacatgcacgaatgcaa

SEQ ID NO: 78 - DNA, 18 bp, Artificial Sequence
  hairpin
tagttatattcaagcata

SEQ ID NO: 79 - DNA, 21 bp, Artificial Sequence
  hairpin
ttgcattcgtgcatgtccgtg

SEQ ID NO: 80 - DNA, 21 bp, Artificial Sequence
  hairpin
gcgaaatctggcgagacatcg

SEQ ID NO: 81 - DNA, 21 bp, Artificial Sequence
  hairpin
ccatattcagcctttgagagt

SEQ ID NO: 82 - DNA, 21 bp, Artificial Sequence
  hairpin
tgtccagaagctaccactgca

SEQ ID NO: 83 - DNA, 18 bp, Artificial Sequence
  hairpin
tagttatattcaagcata

SEQ ID NO: 84 - DNA, 21 bp, Artificial Sequence
  hairpin
tgcagtggtagcttctggaca

SEQ ID NO: 85 - DNA, 21 bp, Artificial Sequence
  hairpin
gcgaaatctggcgagacatcg

SEQ ID NO: 86 - DNA, 102 bp, Artificial Sequence
  hairpin
ccatattcagcctttgagagtcaacgtggacgttcagttcaatagttatattcaagcatattgaactgaacgtccacgttggcgaaatctggcgagacatcg

SEQ ID NO: 87 - DNA, 102 bp, Artificial Sequence
  hairpin
ccatattcagcctttgagagtctcaaagttgtgcaactcttatagttatattcaagcatataagagttgcacaactttgaggcgaaatctggcgagacatcg

SEQ ID NO: 88 - DNA, 102 bp, Artificial Sequence
  hairpin
ccatattcagcctttgagagttccgatgacaatacacttaaatagttatattcaagcatatttaagtgtattgtcatcggagcgaaatctggcgagacatcg

SEQ ID NO: 89 - DNA, 102 bp, Artificial Sequence
  hairpin
ccatattcagcctttgagagttacgacgaggataccattctatagttatattcaagcatatagaatggtatcctcgtcgtagcgaaatctggcgagacatcg

SEQ ID NO: 90 - DNA, 102 bp, Artificial Sequence
  hairpin
ccatattcagcctttgagagtctggctaagtgtggacagaaatagttatattcaagcatatttctgtccacacttagccaggcgaaatctggcgagacatcg

SEQ ID NO: 91 - DNA, 102 bp, Artificial Sequence
  hairpin
ccatattcagcctttgagagtaaggatgtaaagtgaacacaatagttatattcaagcatattgtgttcactttacatccttgcgaaatctggcgagacatcg

SEQ ID NO: 92 - DNA, 102 bp, Artificial Sequence
  hairpin
ccatattcagcctttgagagtctcaccgagacttcaagtctatagttatattcaagcatatagacttgaagtctcggtgaggcgaaatctggcgagacatcg

SEQ ID NO: 93 - DNA, 102 bp, Artificial Sequence
  hairpin
ccatattcagcctttgagagtaagagtgcaaatagaatagaatagttatattcaagcatattctattctatttgcactcttgcgaaatctggcgagacatcg

SEQ ID NO: 94 - DNA, 102 bp, Artificial Sequence
  hairpin
ccatattcagcctttgagagtagcgagaatcccaacagtgtatagttatattcaagcatatacactgttgggattctcgctgcgaaatctggcgagacatcg

SEQ ID NO: 95 - DNA, 102 bp, Artificial Sequence
  hairpin
ccatattcagcctttgagagtctggacgaccagcttcgatgatagttatattcaagcatatcatcgaagctggtcgtccaggcgaaatctggcgagacatcg

SEQ ID NO: 96 - DNA, 102 bp, Artificial Sequence
  hairpin
ccatattcagcctttgagagtaagcctttgtatctatgtcaatagttatattcaagcatattgacatagatacaaaggcttgcgaaatctggcgagacatcg

SEQ ID NO: 97 - DNA, 102 bp, Artificial Sequence
  hairpin
ccatattcagcctttgagagtctcgaagcatcagcaccttaatagttatattcaagcatattaaggtgctgatgcttcgaggcgaaatctggcgagacatcg

SEQ ID NO: 98 - DNA, 102 bp, Artificial Sequence
  hairpin
ccatattcagcctttgagagtaggtatgcgggatcactacgatagttatattcaagcatatcgtagtgatcccgcatacctgcgaaatctggcgagacatcg

SEQ ID NO: 99 - DNA, 102 bp, Artificial Sequence
  hairpin
ccatattcagcctttgagagtatcggtctgctggacatattctagttatattcaagcatagaatatgtccagcagaccgatgcgaaatctggcgagacatcg

SEQ ID NO: 100 - DNA, 102 bp, Artificial Sequence
  hairpin
ccatattcagcctttgagagtcacggacatgcacgaatgcaatagttatattcaagcatattgcattcgtgcatgtccgtggcgaaatctggcgagacatcg

SEQ ID NO: 101 - DNA, 102 bp, Artificial Sequence
  hairpin
ccatattcagcctttgagagttgtccagaagctaccactgcatagttatattcaagcatatgcagtggtagcttctggacagcgaaatctggcgagacatcg

SEQ ID NO: 102 - DNA, 39 bp, Artificial Sequence
  spacer
actctgaatagggaattgggaattgagatctgttctaga

SEQ ID NO: 103 - DNA, 39 bp, Artificial Sequence
  spacer
agtagtgccaccaaaagttagccgcgttgtggaaaatcc

SEQ ID NO: 104 - DNA, 39 bp, Artificial Sequence
  spacer
gagggaaatggagaacgcaaaaatcccattataatggaa

SEQ ID NO: 105 - DNA, 39 bp, Artificial Sequence
  spacer
atgtgcttgatcgtaactccatccaaactcgatattaac

SEQ ID NO: 106 - DNA, 39 bp, Artificial Sequence
  spacer
acaaataatgttgcaataaccagttgaaaccaatggaat

SEQ ID NO: 107 - DNA, 39 bp, Artificial Sequence
  spacer
aactaacccgttcacctgcgacaatttttaatctatttt

SEQ ID NO: 108 - DNA, 39 bp, Artificial Sequence
  spacer
agaccacgatcgaaagaggaaaaacggaaaacgaacgaa

SEQ ID NO: 109 - DNA, 65 bp, Artificial Sequence
  spacer
ggactagttttcattatttatcagccagcaccaacaacatagcggccgcaagaattcaggcgaga

SEQ ID NO: 110 - DNA, 39 bp, Artificial Sequence; identical to SEQ ID NO: 102
  cluster

SEQ ID NO: 111 - DNA, 102 bp, Artificial Sequence; identical to SEQ ID NO: 86
  cluster

SEQ ID NO: 112 - DNA, 39 bp, Artificial Sequence; identical to SEQ ID NO: 103
  cluster

SEQ ID NO: 113 - DNA, 102 bp, Artificial Sequence; identical to SEQ ID NO: 87
  cluster

SEQ ID NO: 114 - DNA, 39 bp, Artificial Sequence; identical to SEQ ID NO: 104
  cluster

SEQ ID NO: 115 - DNA, 102 bp, Artificial Sequence; identical to SEQ ID NO: 88
  cluster

SEQ ID NO: 116 - DNA, 39 bp, Artificial Sequence; identical to SEQ ID NO: 105
  cluster

SEQ ID NO: 117 - DNA, 39 bp, Artificial Sequence; identical to SEQ ID NO: 102
  cluster

SEQ ID NO: 118 - DNA, 102 bp, Artificial Sequence; identical to SEQ ID NO: 94
  cluster

SEQ ID NO: 119 - DNA, 39 bp, Artificial Sequence; identical to SEQ ID NO: 103
  cluster

SEQ ID NO: 120 - DNA, 102 bp, Artificial Sequence; identical to SEQ ID NO: 95
  cluster

SEQ ID NO: 121 - DNA, 39 bp, Artificial Sequence; identical to SEQ ID NO: 104
  cluster

SEQ ID NO: 122 - DNA, 102 bp, Artificial Sequence; identical to SEQ ID NO: 96
  cluster

SEQ ID NO: 123 - DNA, 39 bp, Artificial Sequence; identical to SEQ ID NO: 105
  cluster

SEQ ID NO: 124 - DNA, 102 bp, Artificial Sequence; identical to SEQ ID NO: 89
  cluster

SEQ ID NO: 125 - DNA, 39 bp, Artificial Sequence; identical to SEQ ID NO: 107
  cluster

SEQ ID NO: 126 - DNA, 102 bp, Artificial Sequence; identical to SEQ ID NO: 90
  cluster

SEQ ID NO: 127 - DNA, 39 bp, Artificial Sequence; identical to SEQ ID NO: 108
  cluster

SEQ ID NO: 128 - DNA, 102 bp, Artificial Sequence; identical to SEQ ID NO: 91
  cluster

SEQ ID NO: 129 - DNA, 39 bp, Artificial Sequence
  cluster
ggactagttttcattatttatcagccagcaccaacaaca

SEQ ID NO: 130 - DNA, 102 bp, Artificial Sequence; identical to SEQ ID NO: 92
  cluster

SEQ ID NO: 131 - DNA, 102 bp, Artificial Sequence; identical to SEQ ID NO: 97
  cluster

SEQ ID NO: 132 - DNA, 39 bp, Artificial Sequence; identical to SEQ ID NO: 107
  cluster

SEQ ID NO: 133 - DNA, 102 bp, Artificial Sequence; identical to SEQ ID NO: 98
  cluster

SEQ ID NO: 134 - DNA, 39 bp, Artificial Sequence; identical to SEQ ID NO: 108
  cluster

SEQ ID NO: 135 - DNA, 102 bp, Artificial Sequence; identical to SEQ ID NO: 99
  cluster

SEQ ID NO: 136 - DNA, 39 bp, Artificial Sequence; identical to SEQ ID NO: 129
  cluster

SEQ ID NO: 137 - DNA, 102 bp, Artificial Sequence; identical to SEQ ID NO: 100
  cluster

SEQ ID NO: 138 - DNA, 39 bp, Artificial Sequence; identical to SEQ ID NO: 104
  cluster

SEQ ID NO: 139 - DNA, 102 bp, Artificial Sequence; identical to SEQ ID NO: 93
  cluster

SEQ ID NO: 140 - DNA, 39 bp, Artificial Sequence; identical to SEQ ID NO: 106
  cluster

SEQ ID NO: 141 - DNA, 26 bp, Artificial Sequence
  cluster
tagcggccgcaagaattcaggcgaga

SEQ ID NO: 142 - DNA, 39 bp, Artificial Sequence; identical to SEQ ID NO: 104
  cluster

SEQ ID NO: 143 - DNA, 102 bp, Artificial Sequence; identical to SEQ ID NO: 101
  cluster

SEQ ID NO: 144 - DNA, 39 bp, Artificial Sequence; identical to SEQ ID NO: 106
  cluster

SEQ ID NO: 145 - DNA, 26 bp, Artificial Sequence
  cluster
tagcggccgcaagaattcaggcgaga

SEQ ID NO: 146 - DNA, 1193 bp, Artificial Sequence
  cluster
actctgaatagggaattgggaattgagatctgttctagaccatattcagcctttgagagtcaacgtggacgttcagttcaatagttatattcaagcatattgaactgaacgtccacgttggcgaaatctggcgagacatcgagtagtgccaccaaaagttagccgcgttgtggaaaatccccatattcagcctttgagagtctcaaagttgtgcaactcttatagttatattcaagcatataagagttgcacaactttgaggcgaaatctggcgagacatcggagggaaatggagaacgcaaaaatcccattataatggaaccatattcagcctttgagagttccgatgacaatacacttaaatagttatattcaagcatatttaagtgtattgtcatcggagcgaaatctggcgagacatcgatgtgcttgatcgtaactccatccaaactcgatattaacccatattcagcctttgagagttacgacgaggataccattctatagttatattcaagcatatagaatggtatcctcgtcgtagcgaaatctggcgagacatcgaactaacccgttcacctgcgacaatttttaatctattttccatattcagcctttgagagtctggctaagtgtggacagaaatagttatattcaagcatatttctgtccacacttagccaggcgaaatctggcgagacatcgagaccacgatcgaaagaggaaaaacggaaaacgaacgaaccatattcagcctttgagagtaaggatgtaaagtgaacacaatagttatattcaagcatattgtgttcactttacatccttgcgaaatctggcgagacatcgggactagttttcattatttatcagccagcaccaacaacaccatattcagcctttgagagtctcaccgagacttcaagtctatagttatattcaagcatatagacttgaagtctcggtgaggcgaaatctggcgagacatcggagggaaatggagaacgcaaaaatcccattataatggaaccatattcagcctttgagagtaagagtgcaaatagaatagaatagttatattcaagcatattctattctatttgcactcttgcgaaatctggcgagacatcgacaaataatgttgcaataaccagttgaaaccaatggaattagcggccgcaagaattcaggcgaga

SEQ ID NO: 147 - DNA, 1193 bp, Artificial Sequence
  cluster
actctgaatagggaattgggaattgagatctgttctagaccatattcagcctttgagagtagcgagaatcccaacagtgtatagttatattcaagcatatacactgttgggattctcgctgcgaaatctggcgagacatcgagtagtgccaccaaaagttagccgcgttgtggaaaatccccatattcagcctttgagagtctggacgaccagcttcgatgatagttatattcaagcatatcatcgaagctggtcgtccaggcgaaatctggcgagacatcggagggaaatggagaacgcaaaaatcccattataatggaaccatattcagcctttgagagtaagcctttgtatctatgtcaatagttatattcaagcatattgacatagatacaaaggcttgcgaaatctggcgagacatcgatgtgcttgatcgtaactccatccaaactcgatattaacccatattcagcctttgagagtctcgaagcatcagcaccttaatagttatattcaagcatattaaggtgctgatgcttcgaggcgaaatctggcgagacatcgaactaacccgttcacctgcgacaatttttaatctattttccatattcagcctttgagagtaggtatgcgggatcactacgatagttatattcaagcatatcgtagtgatcccgcatacctgcgaaatctggcgagacatcgagaccacgatcgaaagaggaaaaacggaaaacgaacgaaccatattcagcctttgagagtatcggtctgctggacatattctagttatattcaagcatagaatatgtccagcagaccgatgcgaaatctggcgagacatcgggactagttttcattatttatcagccagcaccaacaacaccatattcagcctttgagagtcacggacatgcacgaatgcaatagttatattcaagcatattgcattcgtgcatgtccgtggcgaaatctggcgagacatcggagggaaatggagaacgcaaaaatcccattataatggaaccatattcagcctttgagagttgtccagaagctaccactgcatagttatattcaagcatatgcagtggtagcttctggacagcgaaatctggcgagacatcgacaaataatgttgcaataaccagttgaaaccaatggaattagcggccgcaagaattcaggcgaga